(19) United States
(12) Patent Application Publication
(10) Pub. No.: US 2011/0262983 A1
(43) Pub. Date: Oct. 27, 2011

(54) METABOLICALLY ENGINEERED YEASTS FOR THE PRODUCTION OF ETHANOL AND OTHER PRODUCTS FROM XYLOSE AND CELLOBIOSE

(75) Inventors: Thomas W. Jeffries, Madison, WI (US); Shawn S. Nelson, Sun Prairie, WI (US); Sarah D. Mahan, Hustisford, WI (US)

(73) Assignee: The United States of America as represented by the Secretary of Agriculture, Washington, DC (US)

(21) Appl. No.: 13/077,653

(22) Filed: Mar. 31, 2011

Related U.S. Application Data
(60) Provisional application No. 61/319,851, filed on Mar. 31, 2010.
(60) Provisional application No. 61/325,181, filed on Apr. 16, 2010.

(51) Int. Cl. C12P 7/06; C12N 1/19

(52) U.S. Cl. 435/161; 435/254.23; 435/254.2

(57) ABSTRACT

The present invention provides yeast cells that produce high concentrations of ethanol, culture media and bioreactors comprising the yeast cells, and methods for making and using the yeast cells in efficiently producing ethanol.

### CROSS-REFERENCES TO RELATED APPLICATIONS

The present application claims the benefit of U.S. Provisional Application No. 61/319,851, filed on Mar. 31, 2010, and U.S. Provisional Application No. 61/325,181, filed on Apr. 16, 2010, the entire disclosures of which are hereby incorporated herein by reference.

### FIELD OF THE INVENTION

The invention relates to the field of industrial microbiology and the production of alcohols. More specifically, ethanol is produced from xylose, glucose, cellobiose and mixtures of sugars in acid and enzymatic hydrolysates via industrial fermentation by a recombinant yeast.

### BACKGROUND OF THE INVENTION

Ethanol obtained from the fermentation of starch from grains or sucrose from sugar cane is being blended with gasoline to supplement petroleum supplies. The relatively oxygenated ethanol increases the efficiency of combustion and the octane value of the fuel mixture. Production of ethanol from grain and other foodstuffs, however, can limit the amount of agricultural land available for food and feed production, thereby leading to the expansion of agricultural production into forests or marginal lands. Moreover, the intense tillage and fertilization of prime agricultural land can result in excessive soil erosion and runoff or penetration of excess phosphorous and nitrogen into waterways and aquifers. Production of ethanol from feedstocks that do not compete with food and animal feed supplies is therefore highly desirous, indeed essential for the large-scale development of renewable fuels from biomass.

Lignocellulosic materials from agricultural residues, fast-growing hardwoods and processing byproducts constitute a large domestic renewable resource that could be used in a sustainable manner for the production of renewable fuels. Substrates presently available in or adjacent to existing grain and sucrose fermentation facilities include grain hulls, corn cobs, corn stalks (stover), sugarcane bagasse, wheat straws various annual or perennial grasses such as Miscanthus species, Sorghum species, giant reed (Arundo donax), and switchgrass (Panicum virgatum), and fast-growing hardwoods such as species of Populus, Sailix and Acer.

Sugars, lignin and various other components can be extracted from these feedstocks following appropriate mechanical, chemical, thermal or other pretreatments. These include the use of heat, steam dilute and concentrated acids or bases, and organic solvents either alone, sequentially to or in combination with mechanical maceration. The pretreatment processes result in the formation of soluble hemicellulosic sugars and oligomeric materials along with partially degraded cellulose, hemicellulose and lignin. Ideally, pretreatments minimize substrate losses and byproduct toxin formation while maximizing the production of sugars available for fermentation.

Sugars can be present in the form of monosaccharides such as D-glucose, D-xylose, D-mannose, D-galactose and L-arabinose or as various oligomers or polymers of these constituents along with other lignocellulosic components such as acetic acid, 4-O-methylglucuronic acid, and ferulic acid. From angiosperms the prevalent sugar polymers are cellulose and xylan, which can be hydrolyzed to glucose and xylose, respectively.

Glucose in sugar hydrolysates represses the induction of transcripts for proteins essential for the assimilation of less readily utilized sugars present in hydrolysates such as xylose, cellobiose, galactose, arabinose, and rhamnose. The production of ethanol from glucose can attain inhibitory concentrations even before use of other sugars commences. Even in cells that normally metabolize and ferment sugars other than glucose, it is therefore desirable to alter the expression of transcripts for the proteins mediating their assimilation so that their utilization starts while glucose is still present.

If an organism is capable of metabolizing other non-carbohydrate components of hemicellulose hydrolysates such as acetic, ferulic, and 4-O-methylglucuronic acids, furfural, hydroxymethyl furfural, and various degradation products of lignin, induction of transcripts for their consumption can likewise be inhibited by the presence of glucose or other more readily utilized carbon sources.

Genes coding for metabolism of xylose, arabinose, mannose, rhamnose or other substrates such as cellobiose, xylan, or glucan can be present in the genome but not expressed at sufficient levels for optimal substrate uptake or product formation. This is especially true of fermentation processes that require a high glycolytic flux. By altering the expression of genes critical for substrate uptake or product formation, it is possible to obtain significantly higher rates of fermentation.

Sugar transport is critical for efficient metabolism during fermentation. For example, it is well known that Saccharomyces cerevisiae, which is highly fermentative, expresses numerous proteins for the uptake of glucose and fructose by facilitated diffusion (1, 6, 9). Several researchers have previously engineered S. cerevisiae for improved xylose utilization by overexpressing the principal glucose/xylose facilitative transporter from Pichia stipitis in S. cerevisiae (5, 11). In the study by Katahira et al., overexpression of SUT1 in S. cerevisiae increased the uptake rate for xylose or glucose in S. cerevisiae cells that had been engineered for xylose metabolism. They were able to achieve 41.4 g/l ethanol with an overall yield of 4.42 g ethanol/g total sugars within 72 h from a mixture of 51.8 g/l glucose and 52.3 g/l xylose. However, the rate and yield of ethanol production from xylose were much lower than from glucose, and approximately 10% of the xylose (5 g/l) remained unused after 72 h. When xylose was the sole carbon source, utilization was better but still incomplete (5).

Proteins that mediate sugar uptake are known to exhibit significant variability even with minor changes in amino acid sequence. For example, Weirstall et al. (11), first cloned and characterized SUT1, SUT2 and SUT3 from P. stipitis, and showed that all three proteins could mediate glucose and xylose transport when expressed in S. cerevisiae. Sut1p differs significantly from Sut2p and Sut3p, whereas Sut2p and Sut3p show only a single amino acid difference (and Sut4p, which was not described by Weirstall et al.). Even so, Sut1p and Sut3p, but not Sut2p were able to mediate significant fructose uptake, but Sut2p could not. Moreover, galactose was taken up only by Sut3, but only in small amounts and with a relatively high Kₘ.

Jeffries et al have shown that the facilitative sugar transporter, Sut4p, shows relatively high affinity for D-xylose as compared to D-glucose, and that it can dramatically increase xylose and glucose utilization when overexpressed in its native host, thereby indicating that sugar transport is rate limiting in this organism. Moreover, Jeffries et al. disclosed that the sugar symporter, Xut1p, exhibits relatively high and selective affinity for D-xylose.

Xylose uptake transporters have been described. Pichia stipitis Xut3p is similar in structure to Pyrenophora tritici-xylose-proton symporter, Xps1p (GenBank REFSEQ: accession XM_{—}001935846.1) and to Debaryomyces hansenii Xylhp (GenBank REFSEQ: accession AY347871.1) and D. hansenii XM_{—}458169.1.

As previously shown by Jin et al. (4) (see also, U.S. Pat. No. 7,226,735) optimal expression of a gene for metabolic pathway engineering does not necessarily require maximal expression as could be obtained through the use of strong constitutive promoters. More appropriate promoters native to the Pichia stipitis genome but exhibiting lower level or expression profiles that vary with the growth condition may be obtained from the published genome of Pichia stipitis: on the internet at genome.jgi-psf.org/Picst3/Picst3.home.html and their expression levels may be determined by Southern hybridization, qPCR, or expression array technologies. As has been demonstrated by Lu et al. (8), the levels of enzymatic activities obtained with promoters native to the host correlate significantly with the transcript level. Thus expression of genes and combinations of genes useful to maximize metabolite flux for desired products can be optimized.

Yeasts such as Saccharomyces cerevisiae and bacteria such as Escherichia coli, Zymomonas mobilis and Klebsiella oxytoca have been engineered for the utilization of xylose and arabinose, but these organisms are limited either by low production rates, strong preference for utilization of glucose over xylose susceptibility to inhibitors, susceptibility to microbial or bacteriophage contamination, high requirements for nutrients, or containment regulations due to the expression of transgenes in order to achieve xylose or cellobiose utilization. There remains a need for yeasts that will ferment glucose, xylose, cellobiose and other sugars from lignocellulosic materials at high rates and yields without these drawbacks.

### BRIEF SUMMARY OF THE INVENTION

The present invention relates to the altered expression of genes in native xylose and cellobiose fermenting yeasts to create novel strains for the more rapid and efficient fermentation of xylose and cellobiose to ethanol wherein the native or previously engineered yeast strains are transformed with individual or multiple genes driven by selected promoters, each of which is native to the host, but which is re-introduced and integrated into the genome in non-native promoter-gene combinations, frequencies or genome locations.

The invention provides a recombinant organism having engineered pathways for xylose, glucose, rhamnose, arabinose and cellobiose metabolism such that the organism can be used for the commercial production of ethanol from mixed sugars, e.g., present in acid and enzymatic hydrolysates of pretreated lignocellulosic materials. Accordingly, referring to FIG. 1, in one embodiment, the invention provides a recombinant yeast cell comprising at least one DNA molecule encoding a polypeptide that catalyzes a substrate to product conversion selected from the group consisting of:
A. enzymatic hydrolysis of beta-1,4-D-glucan (Pathway 1 step A)
B. enzymatic hydrolysis of beta-1,4-D-xylan (Pathway 1 step B)
C. facilitated transport of xylose and glucose (Pathway 1 step C)
D. symport uptake of xylose and glucose (Pathway 1 step D)
E. transport of cellobiose (Pathway 1 step E)
F. enzymatic hydrolysis of cellobiose to glucose (Pathway 1 step F)
G. xylose reduction to xylitol (Pathway 1 step G)
H. xylitol oxidation to xylulose (Pathway 1 step H)
I. The phosphorylation of xylulose to form xylulose 5-phosphate (Pathway 1, step I)
J. The conversion of xylulose-5-phosphate to ribulose-5 phosphate (Pathway 1, step J)
K. The conversion of ribulose 5-phosphate to ribose 5-phosphate (Pathway 1, step K)
L. The conversion of xylulose 5-phosphate and one molecule of ribose 5-phosphate into glyceraldehyde 3-phosphate and sedoheptulose 7-phosphate (Pathway 1, step L)
M. The conversion of sedoheptulose 7-phosphate and glyceraldehyde 3-phosphate into fructose 6-phosphate and erythrose 4-phosphate (Pathway 1, step M)
N. The conversion of xylulose 5-phosphate and erythrose 4-phosphate into fructose-6-phosphate and glyceraldehyde 3-phosphate (Pathway 1, step N)
O. The decarboxylation of pyruvate to acetaldehyde (Pathway 1, step O)
P. The reduction of acetaldehyde to ethanol (Pathway 1, step P)
Q. The oxidation of acetaldehyde to acetate (Pathway 1, step Q).

The invention provides a recombinant yeast that produces ethanol from glucose or xylose with a yield of at least 0.32 g ethanol/g sugar consumed and with a final concentration of at least 50 g ethanol/1 and an ethanol production rate of at least 0.5 g/l·h (grams per liter per hour). Such cells exhibit increased production of ethanol and decreased production of xylitol byproduct when compared to the parental or wild-type strains from which they are derived such that the xylitol yield is less than 0.04 g xylitol/g xylose consumed. The parental or wild type strains may produce ethanol naturally from xylose or cellobiose or they may be engineered to do so.

Accordingly, the invention provides a recombinant yeast cell producing ethanol from xylose or cellobiose wherein at least one genetic modification increases the fermentation rate or yield from xylose or cellobiose or a mixture of at least one of these sugars with glucose.

In one embodiment the yeast cell of the invention comprises a genetic modification in a gene encoding a protein selected from Sut4p, Xut1p, Xut3p, Hxt4p, ZmAdh1p, Hgt1p, Hgt2p, Xyl1p, Xyl2p, Xyl3p, Hxt2.4p, Egc2p, Bgl5p, Hxt2.2p, Hxt2.5p, Tal1p, Tkt1p, or Hxt2.6p.

In another embodiment the yeast cell of the invention comprises a genetic modification in a gene encoding a protein selected from Sut1p, Sut2p, or Sut3p

In another embodiment the yeast cell of the invention comprises a genetic modification in a gene encoding a protein selected from Bgl1p, Bgl2p, Bgl3p, Bgl4p, Bgl5p, Bgl6p, or Bgl1p.

In another embodiment the yeast cell of the invention comprises a genetic modification in a gene encoding a protein selected from Egc1p, Egc2p, Egc3p, or Xyn1p.

In another embodiment the yeast cell of the invention comprises a genetic modification in a gene encoding a protein selected from Hxt2.1p, Hxt2.2p, Hxt2.3p, Hxt2.4p, Hxt2.5p, or Hxt2.6p.

In another embodiment the yeast cell of the invention comprises a genetic modification in a gene encoding a protein selected from GenBank deposited sequences: PICST_{—}68558 (PsAdh1p) or PICST_{—}27980 (PsAdh2p),

In another embodiment the yeast cell of the invention comprises a genetic modification in a gene encoding a protein selected from GenBank deposited sequences: PICST_{—}88760 (PsAdh3p), PICST_{—}29079 (PsAdh4p), PICST_{—}31312 (PsAdh5p), PICST_{—}34588 (PsAdh6p), PICST_{—}45137 (PsAdh7p).

In another embodiment the yeast cell of the invention comprises a genetic modification in a gene encoding a protein selected from GenBank deposited sequences: PICST_{—}64926 (PsPdc1p), PICST_{—}86443 (PsPdc2p)

In another embodiment the yeast cell of the invention comprises a genetic modification in a gene encoding a protein that is coded for by a synthetic gene selected from sSUT4, sZmADH1, or sNAT1.

In another embodiment, the yeast cell of the invention comprises a genetic modification in a gene such that its native promoter sequence is replaced by a promoter selected from PsACB2, PsXUT1, PsTDH3, PsFAS2, PsZWE1, PsBGL5, PsEGC2, PsHXT2.4, ScALD1, PsCLG1, PsENO1, PsLPD1, Ps LSC1, PsMEP2, PsPGI1, PsTAL1, ScTEF2, PsTKT1, and ScTPI1.

In another embodiment the yeast cell of the invention comprises a genetic modification in a gene such that its native terminator sequence is replaced by a terminator selected from PsACB2, PsXUT1, PsTDH3, PsSUT4, PsFAS2, PsZWE1, PsHXT4, PsBGL5, PsEGC2, PsHXT2.2, PsHXT2.4, PsHXT2.5, PsHXT2.6. ScALD1, PsBGL1, PsBGL2, PsBGL3, PsBGL4, PsBGL6, PsBGL7, PsEGC1, PsEGC3, PsHGT1, PsHGT2, PsHXT2.1, PsHXT2.3, PsTDH3, ScTDH3, ScTEF2, ScTPI1, PsXUT3, PsXYN1, PsSUT1, PsSUT2, and PsSUT3.

In another embodiment the yeast cell recombinantly expresses two or more polypeptides in a pathway, wherein the polypeptide is,
a. Xut1p and Sut4p;
b. Xut1p, Sut4p and Hxt4p;
c. Xyl1p and Xyl2p;
d. Xyl1p, Xyl2p and Xyl3p;
e. Hxt2.4p, Egc2p and Bgl5p;
f. Hxt2.2p, Egc2p and Bgl5p;
g. Sut4p, Xyl1p and Xyl2p;
h. Sut4p, Xyl1p, Xyl2p and Xyl3p;
i. Xut1p, Xyl1p and Xyl2p;
j. Xut1p, Xyl1p, Xyl2p and Xyl3p;
k. Hxt4p, Xyl1p and Xyl2p;
l. Hxt4p, Xyl1p, Xyl2p and Xyl3p;
m. Sut4p, Xut1p, Xyl1p and Xyl2p;
n. Sut4p, Hxt4p, Xyl1p, Xyl2p and Xyl3p;
o. Sut4p, Hxt4p, Xyl1p, Xyl2p and ZmADH1;
p. Sut4p, Hxt4p, Xyl1p, Xyl2p, Xyl3p and ZmADH1;
q. Xut1p, Sut4p, Hxt4p and ZmADH1; and
r. Sut4p, Xyl1p, Xyl2p, Tal1p, Tkt1p

In another embodiment the invention provides a method for the production of ethanol comprising the steps of
a. Providing a recombinant yeast cell which
   i. Produces ethanol from xylose or cellobiose and
   ii. Comprises at least one genetic modification which increases the rate or yield of ethanol production; and
   iii. Ferments glucose and xylose from hydrolysates containing acetic acid.
b. Culturing the strain of (a) under conditions wherein ethanol is produced from xylose or cellobiose.

In a related aspect, the invention provides an isolated yeast comprising a heterologous expression cassette comprising a promoter operably linked to polynucleotide encoding a polypeptide substantially (e.g., at least 60%, 65%, 70%, 75%, 80%, 85%, 90%, 95%, 98%, or 99%) identical to any of SEQ ID NOS: 25-55 and 92-95 (Table 2), wherein the yeast has a higher rate and/or yield of ethanol production in comparison to a control yeast lacking the expression cassette. The yield can be measured in any way accepted in the art, e.g., volumetrically (g/L) or specifically (g/g).

In some embodiments, the polypeptide comprises one of SEQ ID NOS: 25-55 or SEQ ID NOS: 92-94. In some embodiments, the polypeptide is substantially (e.g., at least 60%, 65%, 70%, 75%, 80%, 85%, 90%, 95%, 98%, or 99%) identical to any of SEQ ID NOS: 25-55 and SEQ ID NOS: 92-95 (Table 2). For example, the polypeptide can be substantially (e.g., at least 60%, 65%, 70%, 75%, 80%, 85%, 90%, 95%, 98%, or 99%) identical to any of SEQ ID NOS: 25, 26, 27, 28, 29, 30, 31, 32, 33, 34, 35, 36, 37, 38, 39, 40, 41, 42, 43, 44, 44, 45, 46, 47, 48, 49, 50, 51, 52, 53, 54, 55, 92, 93, or 94.

In some embodiments, the promoter is native to the polynucleotide. In some embodiments, the promoter is heterologous to the polynucleotide.

In some embodiments, the promoter is constitutive or inducible. In some embodiments, the promoter comprises one of SEQ ID NOS: 1-24 (Table 1).

In some embodiments, the yeast comprises two or more expression cassettes, wherein the two or more expression cassettes encode a different polypeptide substantially (e.g., at least 60%, 65%, 70%, 75%, 80%, 85%, 90%, 95%, 98%, or 99%) identical to one of SEQ ID NOS: 25-55, or SEQ ID NOS: 92-94 (Table 2). In some embodiments, the yeast comprises 2, 3, 4, 5, 6, 7, 8, 9, 10 or more expression cassettes, wherein the 2, 3, 4, 5, 6, 7, 8, 9, 10 or more expression cassettes encode a different polypeptide substantially (e.g., at least 60%, 65%, 70%, 75%, 80%, 85%, 90%, 95%, 98%, or 99%) identical to one of SEQ ID NOS: 25-55 or SEQ ID NOS: 92-94. In other embodiments, the expression cassette encodes two or more polypeptides. The two or more polypeptides can be different polypeptides substantially (e.g., at least 60%, 65%, 70%, 75%, 80%, 85%, 90%, 95%, 98%, or 99%) identical to one of SEQ ID NOS: 25-55 or SEQ ID NOS: 92-94.

In some embodiments, the yeast comprises two or more copies of the expression cassette, wherein the two or more expression cassettes encode the same polypeptide, thereby increasing expression of the encoded polypeptide. In some embodiments, the yeast comprises 2, 3, 4, 5, 6, 7, 8, 9, 10 or more copies of the expression cassette, wherein the 2, 3, 4, 5, 6, 7, 8, 9, 10 or more expression cassettes encode the same polypeptide, thereby increasing expression of the encoded polypeptide. In other embodiments, the expression cassette encodes two or more copies of the same or substantially similar polypeptides.

In a further aspect, the invention provides methods of generating ethanol, the method comprising culturing the yeast of the invention, as described herein, in a mixture comprising a sugar under conditions such that the yeast converts the sugar to ethanol. In some embodiments, an ethanol yield of at least about 0.3 g ethanol/g sugar consumed (e.g., at least about 0.4, 0.5, 0.6, 0.7, 0.8 g ethanol/g sugar consumed) is produced. In some embodiments, culture media with ethanol concentrations of at least about 50 g ethanol/l (e.g., at least about 55, 60, 65, 70, 75, 80, 85 g ethanol/l) is produced. In some embodiments, the yeast has an ethanol production rate of at least about 0.5 g/l·h (e.g., at least about 0.5, 0.6, 0.7, 0.8, 0.9, 1.0 g/l·h).

In some embodiments, the sugar converted comprises cellobiose. In some embodiments, the sugar converted is cellobiose.

In some embodiments, the sugar converted comprises xylose. In some embodiments, the sugar converted is xylose.

In some embodiments, the yeast converts the sugar to ethanol in the presence of glucose.

In another aspect, the invention provides a bioreactor containing an aqueous solution, the solution comprising a yeast of the invention, as described herein. In some embodiments, the volume of the solution is at least 100, 500, 1000, or 10,000 liters.

In a further aspect, the invention provides an isolated or substantially purified polypeptide substantially (e.g., at least 60%, 65%, 70%, 75%, 80%, 85%, 90%, 95%, 98%, or 99%) identical to any one of SEQ ID NOS: 38-43, wherein the polypeptide is a cellobiose transporter. In some embodiments, the polypeptide comprises any one of SEQ ID NOS: 38-43.

In a further aspect, the invention provides an isolated polynucleotide encoding a cellobiose transporter polypeptide substantially (e.g., at least 60%, 65%, 70%, 75%, 80%, 85%, 90%, 95%, 98%, or 99%) identical to any one of SEQ ID NOS: 38-44. In some embodiments, the polypeptide comprises any one of SEQ ID NOS: 38-44.

In a related aspect, the invention provides methods of converting cellobiose to ethanol, the method comprising, contacting a mixture comprising cellobiose with a yeast under conditions in which the yeast converts the cellobiose to ethanol, wherein the yeast recombinantly expresses a cellobiose transporter polypeptide substantially (e.g., at least 60%, 65%, 70%, 75%, 80%, 85%, 90%, 95%, 98%, or 99%) identical to any of SEQ ID NOS: 38, 39, 40, 41, 42, 43, or 44. In some embodiments, the polypeptide comprises any of SEQ ID NOS: 38, 39, 40, 41, 42, 43, or 44.

With respect to the compositions and methods, in some embodiments, the yeast is of the genus Saccharomyces or Pichia. In some embodiments, the yeast is of the genus Pichia. In some embodiments, the yeast is a recombinantly altered Pichia stipitis strain NRRL-Y7124. In some embodiments, the yeast is a recombinantly altered Pichia stipitis strain CBS 6054. In some embodiments, the yeast is of the genus Saccharomyces, for example, S. cerevisiae.

In a further aspect, the invention provides an isolated yeast cell, recombinantly expressing:
a. one or more xylose transporters;
b. one or more of a xylose reductase, a xylitol dehydrogenase, and/or a xylulokinase; and optionally
c. a transketolase and/or a transaldolase.

In some embodiments, the invention provides an isolated Pichia stipitis cell, recombinantly expressing:
a. a xylose transporter; and
b. one or more of a xylose reductase, a xylitol dehydrogenase, and/or a xylulokinase.

In other embodiments, the isolated Pichia stipitis cell further recombinantly expresses a transketolase and/or a transaldolase.

In some embodiments, the improved yeast cell comprises two or more expression cassettes, wherein the two or more expression cassettes encode at least one xylose tranporter polypeptide and at least one polypeptide from the xylose assimilation pathway (i.e., one or more of a xylose reductase, a xylitol dehydrogenase, and/or a xylulokinase). Preferably, the improved yeast cell has an ethanol production rate that is higher, e.g., at least about 10%, 20%, 30% higher than a yeast cell that does not recombinantly express the proteins for xylose transport and assimilation. In some embodiments, the improved yeast cell of the strain has an ethanol production rate of at least about 0.5 g/l·h, e.g., at least about 0.5, 0.6, 0.7, 0.8, 0.9, 1.0 g/l·h).

In some embodiments, the yeast cells can convert sugars to ethanol in the presence of concentrations of acetic acid in the range of about 0.05% to about 0.5%, for example, at least about 0.075%, 0.085%, 0.10%, 0.11%, 0.115%, 0.12%, 0.13%, 0.14%, 0.15%, 0.16%, 0.17%, 0.18%, 0.19%, 0.20%, 0.25%, 0.30%, 0.35%, 0.40%, 0.45%, and 0.50%. In other embodiments, the yeast cells can convert sugars to ethanol in the presence of concentrations of acetic acid in the range of about 0.50% to about 5.0%, for example, at least about 0.60%, 0.70%, 0.80%, 0.90%, 1.0%, 1.5%, 2.0%, 2.5%, 3.0%, 3.5%, 4.0%, 4.5%, and 5.0%.

In some embodiments, the xylose transporter is selected from the group consisting of Sut1, Sut2, Sut3, Sut4, Xut1 and Xut3. The xylose transporter can be a Pichia stipitis xylose transporter. The improved yeast cell can recombinantly express 1, 2, 3, 4 or more xylose transporters. When recombinantly expressing multiple transporter proteins, the 2 or more transporters can be the same or different. In some embodiments, the improved yeast cell recombinantly expresses Xut1. In some embodiments, the improved yeast cell recombinantly expresses sSut4. In some embodiments, the improved yeast cell recombinantly expresses two copies of Sut4. In some embodiments, the improved yeast cell recombinantly expresses Xut1 and sSut4. In some embodiments, the improved yeast cell recombinantly expresses Xut1 and Xut3. In some embodiments, the improved yeast cell recombinantly expresses sSut4 and Xut3. In some embodiments, the improved yeast cell recombinantly expresses Xut1, Xut3 and sSut4. In some embodiments, the improved yeast cell recombinantly expresses a xylose transporter that is substantially (e.g., at least 60%, 65%, 70%, 75%, 80%, 85%, 90%, 95%, 98%, or 99%) identical to any one of SEQ ID NOS: 46, 47, 48, 49, 50 or 51.

In some embodiments, the improved yeast cell can optionally recombinantly express a cellobiose transporter. The cellobiose transporter can have substantial identity to a Hxt2 polypeptide from yeast cell, for example, Hxt2.1, Hxt2.2, Hxt2.3, Hxt2.4, Hxt2.5 or Hxt2.6 from yeast cell. In some embodiments, the cellobiose transporter recombinantly expressed has substantial (e.g., at least 60%, 65%, 70%, 75%, 80%, 85%, 90%, 95%, 98%, or 99%) identity to any one of SEQ ID NOS: 38-44. In some embodiments, the cellobiose transporter recombinantly expressed is any one of SEQ ID NOS: 38-44.

In some embodiments, the yeast further recombinantly expresses an endo-1,4-beta-glucanase. In some embodiments, the endo-1,4-beta-glucanase is substantially (e.g., at least 60%, 65%, 70%, 75%, 80%, 85%, 90%, 95%, 98%, or 99%) identical to any of SEQ ID NOs: 33, 34, or 35

In some embodiments, the yeast further recombinantly expresses a beta-glucosidase. In some embodiments, the beta-glucosidase is substantially (e.g., at least 60%, 65%, 70%, 75%, 80%, 85%, 90%, 95%, 98%, or 99%) identical to any of SEQ ID NOs: 26, 27, 28, 29, 30, 31, or 32.

In some embodiments, the improved yeast cell recombinantly expresses two or more of a xylose reductase, a xylitol dehydrogenase, and/or a xylulokinase (i.e., xylose assimilation pathway enzymes). In some embodiments, the improved yeast cell recombinantly expresses all three of a xylose reductase, a xylitol dehydrogenase, and/or a xylulokinase. One, two or three of the xylose assimilation pathway enzymes can be from Pichia stipitis. The xylose reductase can be Xyl1, e.g., from Pichia stipitis. The xylitol dehydrogenase can be a Xyl2, e.g., from Pichia stipitis. The xylulokinase can be Xyl3, e.g., from Pichia stipitis. In some embodiments, the improved yeast cell recombinantly expresses Xyl1 and Xyl2. In some embodiments, the improved yeast cell recombinantly expresses Xyl1 and Xyl3. In some embodiments, the improved yeast cell recombinantly expresses Xyl2 and Xyl3. In some embodiments, the improved yeast cell recombinantly expresses Xyl1, Xyl2 and Xyl3.

In some embodiments, the xylose reductase is substantially identical to SEQ ID NO:52. In some embodiments, the xylose reductase is SEQ ID NO:52. In some embodiments, the xylitol dehydrogenase is substantially identical to SEQ ID NO:53. In some embodiments, the xylitol dehydrogenase is SEQ ID NO:53. In some embodiments, the xylulokinase is substantially identical to SEQ ID NO:54. In some embodiments, the xylulokinase is SEQ ID NO:54. In some embodiments, the xylose reductase is substantially (e.g., at least 60%, 65%, 70%, 75%, 80%, 85%, 90%, 95%, 98%, or 99%) identical to GenBank PICST_{—}89614 (Xyl1p); the xylitol dehydrogenase is substantially (e.g., at least 60%, 65%, 70%, 75%, 80%, 85%, 90%, 95%, 98%, or 99%) identical to GenBank PICST_{—}86924 (PsXyl2p); and the xylulokinase is substantially (e.g., at least 60%, 65%, 70%, 75%, 80%, 85%, 90%, 95%, 98%, or 99%) identical to GenBank PICST_{—}68734 (PsXyl3p) (PsXks1p).

In some embodiments, the improved yeast cell further recombinantly expresses a transketolase. The transketolase can be substantially (e.g., at least 60%, 65%, 70%, 75%, 80%, 85%, 90%, 95%, 98%, or 99%) identical to GenBank EAZ62979 (Tkl2; also known as Dihydroxyacetone synthase (DHAS); SEQ ID NO:92) or GenBank ABN64656 (Tkt1; SEQ ID NO:93). In some embodiments, the improved yeast cells further recombinantly expresses a transaldolase. The transaldolase can be substantially identical to GenBank ABN68690 (PsTal1p; SEQ ID NO:94).

In some embodiments, the improved yeast cells further recombinantly expresses an alcohol dehydrogenase. Yeast cells that recombinantly express one or more alcohol dehydrogenase genes (e.g., an ADH1 gene) will produce relatively more ethanol and relatively less acetate. The alcohol dehydrogenase can have substantial identity to an Adh polypeptide, e.g., from Pichia stipitis or Zymomonas mobilis, for example, Adh1 from Zymomonas mobilis. In some embodiments, the alcohol dehydrogenase recombinantly expressed has substantial (e.g., at least 60%, 65%, 70%, 75%, 80%, 85%, 90%, 95%, 98%, or 99%) identity to SEQ ID NO:25. In some embodiments, the alcohol dehydrogenase recombinantly expressed is SEQ ID NO:25.

In some embodiments, the improved yeast cell recombinantly expresses the xylose transporter Xut1, the xylose reductase Xyl1, the xylitol dehydrogenase Xyl2, and the xylulokinase Xyl3. In some embodiments, the improved yeast cell is Pichia stipitis NRRL Y7124 strain 7124.1.158. The xylose transporter Xut1 can be substantially (e.g., at least 60%, 65%, 70%, 75%, 80%, 85%, 90%, 95%, 98%, or 99%) identical to SEQ ID NO:50; the xylose reductase Xyl1 can be substantially identical to SEQ ID NO:52; the xylitol dehydrogenase Xyl2 can be substantially identical to SEQ ID NO:53; and the xylulokinase Xyl3 can be substantially identical to SEQ ID NO:54.

In some embodiments, the improved yeast cell recombinantly expresses the xylose transporter sSut4, the xylose reductase Xyl1, the xylitol dehydrogenase Xyl2, and the xylulokinase Xyl3. In some embodiments, the improved yeast cell is selected from Pichia stipitis NRRL Y7124 strains 7124.2.415, 7124.2.416, 7124.2.417, 7124.2.418, and 7124.2.419. The xylose transporter sSut4 can be substantially (e.g., at least 60%, 65%, 70%, 75%, 80%, 85%, 90%, 95%, 98%, or 99%) identical to SEQ ID NO:49; the xylose reductase Xyl1 can be substantially identical to SEQ ID NO:52; the xylitol dehydrogenase Xyl2 can be substantially (e.g., at least 60%, 65%, 70%, 75%, 80%, 85%, 90%, 95%, 98%, or 99%) identical to SEQ ID NO:53; and the xylulokinase Xyl3 can be substantially (e.g., at least 60%, 65%, 70%, 75%, 80%, 85%, 90%, 95%, 98%, or 99%) identical to SEQ ID NO:54.

In some embodiments, the improved yeast cell recombinantly expresses two or more copies of the xylose transporter Sut4, and further expresses the xylose reductase Xyl1, the xylitol dehydrogenase Xyl2, and the xylulokinase Xyl3.

In some embodiments, the improved yeast cell recombinantly expresses the xylose transporter sSut4, the xylose reductase Xyl1, and the xylitol dehydrogenase Xyl2. In some embodiments, the xylose transporter sSut4 is substantially (e.g., at least 60%, 65%, 70%, 75%, 80%, 85%, 90%, 95%, 98%, or 99%) identical to SEQ ID NO:49; the xylose reductase Xyl1 is substantially (e.g., at least 60%, 65%, 70%, 75%, 80%, 85%, 90%, 95%, 98%, or 99%) identical to SEQ ID NO:52; and the xylitol dehydrogenase Xyl2 is substantially (e.g., at least 60%, 65%, 70%, 75%, 80%, 85%, 90%, 95%, 98%, or 99%) identical to SEQ ID NO:53.

In some embodiments, the improved yeast cell recombinantly expresses the xylose transporter Sut4, the xylose reductase Xyl1, the xylitol dehydrogenase Xyl2, the transaldolase TAL1 and the transketolase TKT1. In some embodiments, the transaldolase TAL1 is substantially (e.g., at least 60%, 65%, 70%, 75%, 80%, 85%, 90%, 95%, 98%, or 99%) identical to SEQ ID NO:94 and the transketolase TKT1 is substantially (e.g., at least 60%, 65%, 70%, 75%, 80%, 85%, 90%, 95%, 98%, or 99%) identical to SEQ ID NO:93.

In some embodiments, the improved yeast cell is produced by mating a strain that expresses the xylose reductase Xyl1, the xylitol dehydrogenase Xyl2, and the xylulokinase Xyl3 with a strain that expresses the xylose reductase Xyl1, the xylitol dehydrogenase Xyl2, the xylulokinase Xyl3, and at least two copies of the xylose transporter Sut4.

In some embodiments, the improved yeast cell is produced by mating a strain that express the xylose reductase Xyl1, the xylitol dehydrogenase Xyl2, and the xylulokinase Xyl3 with a strain that expresses the xylose transporter sSut4 and 2 copies each of the xylose reductase Xyl1, the xylitol dehydrogenase Xyl2, and the xylulokinase Xyl3.

In a further aspect, the invention further provides methods of converting xylose to ethanol comprising culturing the improved yeast cells described herein. In a related aspect, the invention further provides methods of producing ethanol comprising culturing the improved yeast cells described herein.

In a further aspect, the invention further provides a bioreactor containing an aqueous solution, the solution comprising improved yeast cells, as described herein. In some embodiments, the volume of the solution is at least 100, 500, 1000, 10,000, 20,000, 50,000 or 100,000 liters.

With respect to the compositions and methods, in some embodiments, the yeast is of the genus Saccharomyces or Pichia. In some embodiments, the yeast is of the genus Pichia. In some embodiments, the yeast is a recombinantly altered Pichia stipitis strain NRRL-Y7124. In some embodiments, the yeast is a recombinantly altered Pichia stipitis strain CBS 6054. In some embodiments, the yeast is of the genus Saccharomyces, for example, S. cerevisiae.

The present invention also provides for an isolated yeast cell recombinantly expressing:

a. a cellobiose transporter; and
b. a beta-glucosidase.

In some embodiments, the cellobiose transporter is substantially (e.g., at least 60%, 65%, 70%, 75%, 80%, 85%, 90%, 95%, 98%, or 99%) identical to any of SEQ ID NOs: 38, 39, 40, 41, 42, 43, or 44. In some embodiments, the beta-glucosidase is substantially (e.g., at least 60%, 65%, 70%, 75%, 80%, 85%, 90%, 95%, 98%, or 99%) identical to any of SEQ ID NOs: 26, 27, 28, 29, 30, 31, or 32.

In some embodiments, the yeast further recombinantly expresses:

c. an endo-1,4-beta-glucanase.

In some embodiments, the endo-1,4-beta-glucanase is substantially (e.g., at least 60%, 65%, 70%, 75%, 80%, 85%, 90%, 95%, 98%, or 99%) identical to any of SEQ ID NOs: 33, 34, or 35.

In some embodiments, the yeast is of the genus Saccharomyces or Pichia.

In some embodiments, the yeast utilizes cellobiose at a rate of at least 0.15 g/l per hour.

The present invention also provides for a method of converting cellobiose to ethanol, the method comprising, contacting a mixture comprising cellobiose with a yeast cell recombinantly expressing a cellobiose transporter and a beta-glucosidase under conditions in which the yeast converts the cellobiose to ethanol.

In some embodiments, the yeast also converts a C5 sugar (e.g., xylose) into ethanol.

In a further aspect, the invention further provides a bioreactor containing an aqueous solution, the solution comprising improved yeast cells, as described herein. In some embodiments, the volume of the solution is at least 100, 500, 1000, 10,000, 20,000, 50,000 or 100,000 liters.

The various embodiments of the invention can be more fully understood from the following detailed description, the figures and the accompanying sequence descriptions, which form a part of this application.

### DEFINITIONS

The term “isolated,” when applied to a nucleic acid or protein, denotes that the nucleic acid or protein is essentially free of other cellular components with which it is associated in the natural state. It can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The terms “identical” or percent “identity,” in the context of two or more nucleic acids or polypeptide sequences, refer to two or more sequences or subsequences that are the same. Sequences are “substantially identical” if they have a specified percentage of amino acid residues or nucleotides that are the same (i.e., at least 60% identity, optionally at least 65%, 70%, 75%, 80%, 85%, 90%, 95%, 98%, or 99% identity over a specified region (or the whole reference sequence when not specified)), when compared and aligned for maximum correspondence over a comparison window, or designated region as measured using one of the following sequence comparison algorithms or by manual alignment and visual inspection. The present invention provides for promoters that are substantially identical to any of SEQ ID NOS: 1-24; polypeptides substantially identical to SEQ ID NOS: 25-55 or SEQ ID NOS: 92-94; and polynucleotides substantially identical to SEQ ID NOS:56-91. Optionally, the identity exists over a region that is at least about 50 nucleotides or amino acids in length, or more preferably over a region that is 100 to 500 or 1000 or more nucleotides or amino acids in length, or over the full-length of the sequence.

The term “similarity,” or “percent similarity,” in the context of two or more polypeptide sequences, refer to two or more sequences or subsequences that have a specified percentage of amino acid residues that are either the same or similar as defined in the 8 conservative amino acid substitutions defined above (i.e., 60%, optionally 65%, 70%, 75%, 80%, 85%, 90%, or 95% similar over a specified region), when compared and aligned for maximum correspondence over a comparison window, or designated region as measured using one of the following sequence comparison algorithms or by manual alignment and visual inspection. Sequences having less than 100% similarity but that have at least one of the specified percentages are said to be “substantially similar.” Optionally, this identity exists over a region that is at least about 50 amino acids in length, or more preferably over a region that is at least about 100 to 500 or 1000 or more amino acids in length, or over the full-length of the sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Examples of an algorithm that is suitable for determining percent sequence identity and sequence similarity include the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes or other nucleic acid sequences arranged to make a new functional nucleic acid, e.g., a promoter from one source and a coding region from another source. The term “native” with reference to portions of a nucleic acid indicates that the nucleic acid comprises two or more subsequences that are found in the same relationship to each other in nature.

The term “autologous” when used with reference to portions of a nucleic acid indicates that the nucleic acid occurs in nature in the species. For example, in the present invention nucleic acids naturally occurring in Pichia yeast cells are transformed into and recombinantly expressed in Pichia yeast cells.

An “expression cassette” is a nucleic acid construct, generated recombinantly or synthetically, with a series of specified nucleic acid elements that permit transcription of a particular nucleic acid in a host cell. The expression cassette can optionally be part of a plasmid, virus, or other nucleic acid fragment. Typically, the expression cassette includes promoter operably linked to a nucleic acid to be transcribed.

A “control yeast” refers to an otherwise identical yeast that does not comprise an expression cassette of the invention.

Pichia stipitis strain NRRL Y-7124 has been deposited as ATCC Number 58376.

Pichia stipitis strain CBS 6054 (also known as CCRC 21777, IFO 10063, NRRL Y-11545) has been deposited as ATCC Number 58785.

By “xylose-containing material,” it is meant any medium comprising xylose or oligomeric polymers of xylose, whether liquid or solid. Suitable xylose-containing materials include, but are not limited to, hydrolysates of polysaccharide or lignocellulosic biomass such as corn hulls, wood, paper, agricultural by-products, and the like.

By a “hydrolysate” as used herein, it is meant a polysaccharide that has been depolymerized through the addition of water to form mono and oligosaccharides. Hydrolysates may be produced by enzymatic or acid hydrolysis of the polysaccharide-containing material, by a combination of enzymatic and acid hydrolysis, or by an other suitable means.

### BRIEF DESCRIPTION OF THE DRAWINGS

FIG. 1 shows a metabolic pathway for the assimilation of glucose, xylose, β-1,4-D-glucan, and β-1,4-D-xylan wherein the reactions A through Q are catalyzed by the following:
A. Endoglucanase (Egc1p, Egc2p, Egc3p);
B. Endoxylanase (Egc1p, Egc2p, Egc3p, Xyn1p);
C. Cellobiose transport (Hxt2.1p, Hxt2.2p, Hxt2.3p, Hxt2.4p, Hxt2.5p, Hxt2.6p);
D. Facilitated transport of xylose and glucose (Sut1p, Sut2p, Sut3p, Sut4p, Hxt4p);
E. Symport uptake of xylose and glucose (Xut1p, Xut3p, Hxt4p);
F. β-1,4-cellobiohydrolase (cellobiase) (β-glucosidase) Bgl1p, Bgl2p, Bgl3p, Bgl4p, Bgl5p, Bgl6p;
G. NAD(P)H-dependent D-xylose reductase (aldose reductase) GenBank PICST_{—}89614 (Xyl1p);
H. D-xylulose reductase (xylitol dehydrogenase) GenBank PICST_{—}86924 (PsXyl2p);
I. D-xylulokinase GenBank PICST_{—}68734 (PsXyl3p) (PsXks1p);
J. D-ribulose-5-phosphate 3-epimerase PICST_{—}50761 (PsRpe1p);
K. Ribose-5-phosphate isomerase B (phosphoriboisomerase B) PICST_{—}57049 (PsRPI1);
L. Dihydroxyacetone synthase PICST_{—}53327 (Dha1p) (DHAS) (TKL2) (formaldehyde transketolase), (glycerone synthase); PICST_{—}67105 (PsTkt1p);
M. Transaldolase PICST_{—}74289 (PsTal1p);
N. Dihydroxyacetone synthase PICST_{—}53327 (Dha1p) (DHAS) (TKL2) (Formaldehyde transketolase), (glycerone synthase); PICST_{—}67105 (PsTkt1p);
O. Pyruvate decarboxylase PICST_{—}64926 (PsPdc1p), PICST_{—}86443 (PsPdc2p);
P. Alcohol dehydrogenase PICST_{—}68558 (PsAdh1p), PICST_{—}27980 (PsAdh2p), ZmAdh1p; and
Q. Aldehyde dehydrogenase PICST_{—}29563 (PsAld5p), PICST_{—}28221 (PsAld7p); Q. mitochondrial aldehyde dehydrogenase PICST_{—}63844 (PsAld2p), PICST_{—}60847 (PsAld3p), PICST_{—}80168 (PsAld6p).

FIG. 2 shows the relative rates of glucose and xylose fermentation by the wild-type parental strain Pichia stipitis NRRL Y-7124 and the genetically modified strain P. stipitis Y-7124.1.136, which is expressing a gene encoding Xut1p when both strains are cultivated in shake flasks.

FIG. 3 shows the relative rates of glucose and xylose fermentation by the genetically modified strain Pichia stipitis NRRL Y-7124.1.144, which is expressing proteins encoded for by XUT1 and sSUT4, and the parental strain, P. stipitis Y-7124.1.136 when both strains are cultivated in shake flasks.

FIG. 4 shows the relative rates of glucose and xylose fermentation by the genetically modified strain Pichia stipitis NRRL Y-7124.1.144, which is expressing proteins encoded for by XUT1 and sSUT4, and the parental strain, P. stipitis Y-7124.1.136 when both are cultivated in bioreactors under low aeration conditions, 2% dissolved oxygen with 500 RPM agitation, pH controlled at 5.0, at 25° C.

FIG. 5 shows the relative rates of glucose and xylose fermentation by the wild-type parental strain Pichia stipitis NRRL Y-7124 and the genetically modified strain P. stipitis Y-7124.2.344, which is expressing a pathway [pathway g, discussed above] in which genes for XYL1, and XYL2 and sSUT4 are employed and when both strains are cultivated in shake flasks.

FIG. 6 shows the relative rates of glucose and xylose fermentation by the wild-type parental strain Pichia stipitis NRRL Y-7124 and the genetically modified strain P. stipitis Y-7124.2.344, which is expressing a pathway [pathway g, discussed above] in which genes for XYL1, and XYL2 and sSUT4 are employed and when both strains are cultivated in bioreactors under low aeration conditions, 2% dissolved oxygen with 500 RPM agitation, pH controlled at 5.0, at 25° C.

FIG. 7 shows the relative rates of glucose and xylose fermentations by the wild-type parental strain Pichia stipitis NRRL Y-7124 and the genetically modified strain P. stipitis Y-7124.2.474, which is expressing a pathway [pathway k, discussed above] in which genes for XYL1, XYL2 (also referred to herein as XYL1,2) and HXT4 are employed and when both strains are cultivated in shake flasks.

FIG. 8 shows the glucose utilization rates of the Pichia stipitis NRRL Y-7124, P. stipitis Y-7124.1.136, and the genetically modified P. stipitis strains 7124.1.158, 7124.1.159, 7124.1.160, 7124.1.161, 7124.1.162, 7124.1.163, which are expressing a pathway [pathway j, discussed above] in which genes for XYL1, XYL2, XYL3, (also referred to herein as XYL1,2,3) and XUT1 are employed and when all are cultivated in shake flasks.

FIG. 9 shows the xylose utilization rates of the Pichia stipitis NRRL Y-7124, P. stipitis Y-7124.1.136, and the genetically modified P. stipitis strains 7124.1.158, 7124.1.159, 7124.1.160, 7124.1.161, 7124.1.162, 7124.1.163, which are expressing a pathway [pathway j, discussed above] in which genes for XYL1,2,3, and XUT1 are employed and when all are cultivated in shake flasks.

FIG. 10 shows the ethanol yield of the Pichia stipitis NRRL Y-7124, P. stipitis Y-7124.1.136, and the genetically modified P. stipitis strains 7124.1.158, 7124.1.159, 7124.1.160, 7124.1.161, 7124.1.162, 7124.1.163, which are expressing a pathway (pathway j, discussed above) in which genes for XYL1,2,3, and XUT1 are employed and when all are cultivated in shake flasks.

FIG. 11 shows the ethanol production rates of the Pichia stipitis NRRL Y-7124, P. stipitis Y-7124.1.136, and the genetically modified P. stipitis strains 7124.1.158, 7124.1.159, 7124.1.160, 7124.1.161, 7124.1.162, 7124.1.163, which are expressing a pathway [pathway j, discussed above] in which genes for XYL1,2,3, and XUT1 are employed and when all are cultivated in shake flasks.

FIG. 12 shows the xylitol yield of the Pichia stipitis NRRL Y-7124, P. stipitis Y-7124.1.136, and the genetically modified P. stipitis strains 7124.1.158, 7124.1.159, 7124.1.160, 7124.1.161, 7124.1.162, 7124.1.163, which are expressing a pathway [pathway j, discussed above] in which genes for XYL1,2,3, and XUT1 are employed and when all are cultivated in shake flasks.

FIG. 13 shows the relative rates of glucose and xylose fermentations by the genetically modified strain P. stipitis Y-7124.1.136 and the genetically modified strain Pichia stipitis Y-7124.1.158 which is expressing a pathway [pathway j, discussed above] in which genes for XYL1,2,3 and XUT1 are employed and in which both strains are cultivated in shake flask.

FIG. 14 shows the relative rates of glucose and xylose fermentations by the genetically modified strain Pichia stipitis Y-7124.1.158 and the wild-type parental strain Pichia stipitis NRRL Y-7124 when both are cultivated in bioreactors under low aeration conditions, 10% dissolved oxygen with variable agitation (50-500 RPM), pH controlled at 5.0, at 25° C.

FIG. 15 shows Pichia stipitis Y-7124.1.158 cultivated in bioreactors under two different oxygenation conditions. Condition 1: Cells were cultivated under low aeration conditions, 10% dissolved oxygen with variable agitation (50-500 RPM), pH controlled at 5.0, at 25° C. Condition 2: Cells were cultivated under low aeration conditions, 2% dissolved oxygen with 500 RPM agitation, pH controlled at 5.0, at 25° C.

FIG. 16 shows the relative rates of glucose and xylose fermentations by the wild-type parental strain Pichia stipitis NRRL Y-7124 and the genetically modified strain P. stipitis Y-7124.2.415 which is expressing a pathway [pathway h, discussed above] in which genes for XYL1,2,3 and sSUT4 are employed and in which both strains are cultivated in shake flasks.

FIG. 17 shows Pichia stipitis Y-7124.2.418 cultivated in bioreactors under two different oxygenation conditions. Condition 1: Cells were cultivated under low aeration conditions, 10% dissolved oxygen with variable agitation (50-500 RPM), pH controlled at 5.0, at 25° C. Condition 2: Cells were cultivated under low aeration conditions, 2% dissolved oxygen with 500 RPM agitation, pH controlled at 5.0, at 25° C.

FIG. 18 shows the relative rates of glucose and xylose fermentations by the wild-type parental strain Pichia stipitis NRRL Y-7124 and the genetically modified strain Pichia stipitis Y-7124.2.407 which is expressing a pathway [pathway o, discussed above] in which genes for XYL1, XYL2, sSUT4, HXT4 and sZmADH1 are employed and in which both strains are cultivated in bioreactors under low aeration conditions, 2% dissolved oxygen with 500 RPM agitation, pH controlled at 5.0, at 25° C.

FIG. 19 shows the relative rates of glucose and xylose fermentations by the genetically modified strain Pichia stipitis Y-7124.1.144 and the genetically modified strain Pichia stipitis Y-7124.1.155, which is expressing a pathway [pathway q, discussed above] in which genes for XUT1, sSUT4, HXT4 and sZmADH1 are employed and in which both strains are cultivated in shake flasks.

FIG. 20 shows the relative rates of glucose and xylose fermentations by the wild-type parental strain Pichia stipitis NRRL Y-7124 and the genetically modified strain Pichia stipitis Y-7124.2.462, which is expressing a pathway [pathway p, discussed above] in which genes for XYL1,2,3, sSUT4, HXT4 and sZmADH1 are employed and in which both strains are cultivated in shake flasks.

FIG. 21 shows the sugar utilization rates for Pichia stipitis NRRL Y-7124, and the genetically modified P. stipitis strains 7124.2.465, 7124.2.466, 7124.2.467, 7124.2.468, which are expressing a gene encoding Xut3p, when all strains are cultivated in shake flasks.

FIG. 22 shows the ethanol yield for Pichia stipitis NRRL Y-7124, and the genetically modified P. stipitis strains 7124.2.465, 7124.2.466, 7124.2.467, 7124.2.468, which are expressing a gene encoding Xut3p, when all strains are cultivated in shake flasks.

FIG. 23 shows the specific ethanol yield for Pichia stipitis NRRL Y-7124, and the genetically modified P. stipitis strains 7124.2.465, 7124.2.466, 7124.2.467, 7124.2.468, which are expressing a gene encoding Xut3p, when all strains are cultivated in shake flasks.

FIG. 24 shows the relative rates of growth and ethanol production from cellobiose by the ura3 mutant Pichia stipitis FPL-Y-UC7 and Pichia stipitis FPL-Y-UC7.1.101 genetically modified by the expression of at least one extra copy of HXT2.4, which uses its native promoter, when both strains are cultivated in shake flasks.

FIG. 25 shows the relative rates of growth and ethanol production from cellobiose by the ura3 mutant Pichia stipitis FPL-Y-UC7 and Pichia stipitis FPL-Y-UC7.1.102, which was genetically modified by the expression of at least one extra copy of HXT2.4, EGC2 and BGL5, each of which uses its native promoter, when both strains are cultivated in shake flasks.

FIG. 26 shows the relative rates of growth and ethanol production from cellobiose and glucose by the mutant S. cerevisiae CEN. PK. 111-27B (SSN7) transformed with plasmids pRS424 and pRS425, which carry genes for TRP1 and LEU2, respectively, and S. cerevisiae SSN17, which was genetically modified by the insertion of plasmids pSN261 and pSN259 carrying genes for LEU2, HXT2.2 and TRP1, PsBGL5, respectively.

FIG. 27 shows the relative rates of growth and ethanol production from cellobiose and glucose by the mutant S. cerevisiae CEN. PK. 111-27B (SSN7) transformed with plasmids pRS424 and pRS425, which carry genes for TRP1 and LEU2, respectively, and S. cerevisiae SSN18, which was genetically modified by the insertion of plasmids pSN260 and pSN259 carrying genes for LEU2, HXT2.2 and TRP1, PsBGL5, respectively.

FIG. 28 shows the relative rates of growth and ethanol production from cellobiose and glucose by the mutant S. cerevisiae CEN. PK. 111-27B (SSN7) transformed with plasmids pRS424 and pRS425, which carry genes for TRP1 and LEU2, respectively, and S. cerevisiae SSN21, which was genetically modified by the insertion of plasmids pSN264 and pSN259 carrying genes for LEU2, HXT2.6 and TRP1, PsBGL5.

FIG. 29 shows the relative rates of growth and ethanol production from cellobiose and glucose by the mutant S. cerevisiae CEN. PK. 111-27B (SSN7) transformed with plasmids pRS424 and pRS425, which carry genes for TRP1 and LEU2, respectively, and S. cerevisiae SSN23, which was genetically modified by the insertion of plasmids pSN266 and pSN259, carrying genes for LEU2, HXT2.6 and TRP1, PsBGL5.

FIG. 30 shows the strain development tree of the Y7124 Pichia strains discussed herein.

FIG. 31 shows the effects of overexpression of xylose transport and assimilation genes in Pichia stipitis NRRL Y-7124 strains. Pichia stipitis NRRL Y-7124 strain 7124.1.158 had an ethanol yield that was nearly 40% greater than parent strain NRRL Y-7124 (upper left graph).

FIG. 32 illustrates ethanol production (g/L) of different improved Pichia stipitis NRRL Y-7124 strains under different fermentation conditions in a 3 L bioreactor. The improved Pichia stipitis NRRL Y-7124 strains can produce culture media concentrations of at least about 40 g/L ethanol over about 50 hours.

FIG. 33 illustrates improving fermentative capacity on cellobiose in Pichia stipitis.

FIG. 34 illustrates S. cerevisiae engineered for cellobiose fermentation.

FIG. 35 illustrates the relative fermentation rates for Y-7124 and various independently-obtained clones that were all derived from the same transformation.

FIG. 36 illustrates the abilities of the parental strain Y-7124 and genetically engineered strain Y-7124.2.535 to ferment a filtered hydrolysate of corn stover.

FIG. 37 illustrates the relative fermentation performance of the parental strain Y-7124 and two independent transformant clones before and after the first round of adaptation to hydrolysate.

FIG. 38 illustrates the relative fermentation performance of the parental strain Y-7124 and two independent transformant clones before and after the second round of adaptation to hydrolysate.

FIG. 39 illustrates the relative growth rates of the parental strain Y-7124 and two independent transformant clones before and after the second round of adaptation to hydrolysate.

FIG. 40 illustrates differences in the capacities of Scheffersomyces (Pichia) stipitis CBS 6054 and Y-7124 in the capacities of the native strains to ferment pre-fermented hydrolysate.

FIG. 41 illustrates the crosses between independently derived transformant lines derived from Scheffersomyces (Pichia) stipitis CBS 6054 and Y-7124.

FIG. 42 illustrates the fermentation of Pre-Fermented Corn Stover Hydrolysate Media (0.3% Acetic Acid): 53.6% (v/v) filter-sterilized pre-fermented corn stover hydrolysate supplemented with 6% (w/v) xylose, and 2.4 g/L urea, pH 5.1 by cell lines derived from crosses B, C, D and E.

FIG. 43 illustrates the fermentation of Pre-Fermented Corn Stover Hydrolysate Media (0.3% Acetic Acid): 53.6% (v/v) filter-sterilized pre-fermented corn stover hydrolysate supplemented with 6% (w/v) xylose, and 2.4 g/L urea, pH 5.1 by cell lines derived from crosses F, G and H and CBS 6054.

### DETAILED DESCRIPTION

### I. Introduction

The present invention provides yeast cells that produce high concentrations of ethanol, culture media and bioreactors comprising the yeast cells, and methods for making and using the yeast cells in efficiently producing ethanol. The yeast cells are modified to express multiple copies of native enzymes and/or transporters or copies of heterologous enzymes and/or transporters involved in the metabolic pathway for the transport and assimilation of sugars, e.g., xylose and/or cellobiose. In particular, the yeast cells are modified to recombinantly express a xylose transporter in combination with enzymes that metabolize xylose (e.g., reduction, oxidation and/or phosphorylaton of xylose); optionally a cellobiose transporter, e.g., in combination with one or more enzymes that metabolize cellobiose; and optionally also transketolase and transaldolase enzymes. The improved yeast cells may also recombinantly express an alcohol dehydrogenase.

In some embodiments, the modified yeast cells can constitutively metabolize xylose to produce ethanol in the presence of glucose, thereby allowing for the production of ethanol by concurrently metabolizing at least two sources of sugar. The yeast cells of the invention can produce ethanol with a yield of at least about 0.3 g ethanol/g sugar consumed (e.g., at least about 0.4, 0.5, 0.6, 0.7, 0.8 g ethanol/g sugar consumed); culture media with ethanol concentrations of at least about 50 g ethanol/l (e.g., at least about 55, 60, 65, 70, 75, 80, 85 g ethanol/1) and can have an ethanol production rate of at least about 0.5 g/l·h (e.g., at least about 0.5, 0.6, 0.7, 0.8, 0.9, 1.0 g/l·h).

Moreover, it has been discovered that the Pichia stipitis stains, and in particular, Pichia stipitis NRRL Y-7124 strain, deposited as ATCC Number 58376, is well suited to the production of high specific yields of ethanol. Therefore, the present invention provides numerous high ethanol producing variations of the Pichia stipitis (e.g., Pichia stipitis NRRL Y-7124) background engineered to recombinantly express one or more xylose transporters and one or more enzymes in the xylose assimilation pathway; optionally also one or more cellobiose transporters and one or more enzymes in the cellobiose metabolism pathway; optionally also a transketolase and/or transaldolase enzyme; and optionally also an alcohol dehydrogenase.

### II. Summary of Sequences and Yeast Strains

TABLE 1
Summary of promoter sequences used this study

| Description | SEQ ID NO: Nucleic acid |
|---|---|
| PICST_37097 from Pichia stipitis | 1 |
| PICST_84653 from Pichia stipitis | 2 |
| ACB2 from Pichia stipitis | 3 |
| ALD1 from Saccharomyces cerevisiae | 4 |
| BGL5 from Pichia stipitis | 5 |
| CLG1 from Pichia stipitis | 6 |
| EGC2 from Pichia stipitis | 7 |
| ENO1 from Pichia stipitis | 8 |
| FAS2 from Pichia stipitis | 9 |
| HXT2.4 from Pichia stipitis | 10 |
| LPD1 from Pichia stipitis | 11 |
| LSC1 from Pichia stipitis | 12 |
| MEP2 from Pichia stipitis | 13 |
| PGI1 from Pichia stipitis | 14 |
| TAL1 from Pichia stipitis | 15 |
| TDH3 from Pichia stipitis | 16 and 17 |
| TDH3 from Saccharomyces cerevisiae | 18 and 19 |
| TEF2 from Saccharomyces cerevisiae | 20 |
| TKT1 from Pichia stipitis | 21 |
| TPI1 from Saccharomyces cerevisiae | 22 |
| XUT1 from Pichia stipitis | 23 |
| ZWF1 from Pichia stipitis | 24 |

TABLE 2
Summary of protein sequences used this study

| | | SEQ ID NO: |
|---|---|---|
| Description | Function | Peptide |
| ADH1 from Zymomonas | alcohol dehydrogenase | 25 |
| mobilis | | |
| BGL1 from Pichia stipitis | beta-glucosidase | 26 |
| BGL2 from Pichia stipitis | beta-glucosidase | 27 |
| BGL3 from Pichia stipitis | beta-glucosidase | 28 |
| BGL4 from Pichia stipitis | beta-glucosidase | 29 |
| BGL5 from Pichia stipitis | beta-glucosidase | 30 |
| BGL6 from Pichia stipitis | beta-glucosidase | 31 |
| BGL7 from Pichia stipitis | beta-glucosidase | 32 |
| EGC1 from Pichia stipitis | endo-1,4-beta-glucanase | 33 |
| EGC2 from Pichia stipitis | endo-1,4-beta-glucanase | 34 |
| EGC3 from Pichia stipitis | endo-1,4-beta-glucanase | 35 |
| HGT1 from Pichia stipitis | glucose transporter | 36 |
| HGT2 from Pichia stipitis | glucose transporter | 37 |
| HXT2.1 from Pichia stipitis | cellobiose transporter | 38 |
| HXT2.2 from Pichia stipitis | cellobiose transporter | 39 |
| HXT2.3 from Pichia stipitis | cellobiose transporter | 40 |
| HXT2.4 from Pichia stipitis | cellobiose transporter | 41 |
| HXT2.5 from Pichia stipitis | cellobiose transporter | 42 |
| HXT2.6 from Pichia stipitis | cellobiose transporter | 43 |
| HXT4 from Pichia stipitis | cellobiose transporter | 44 |
| NAT1 from Streptomyces | Nourseothricin resistance | 45 |
| noursei | | |
| SUT1 from Pichia stipitis | glucose/xylose transporter | 46 |
| SUT2 from Pichia stipitis | glucose/xylose transporter | 47 |
| SUT3 from Pichia stipitis | glucose/xylose transporter | 48 |
| SUT4 from Pichia stipitis | glucose/xylose transporter | 49 |
| XUT1 from Pichia stipitis | xylose transporter | 50 |
| XUT3 from Pichia stipitis | xylose transporter | 51 |
| XYL1 from Pichia stipitis | xylose reductase | 52 |
| XYL2 from Pichia stipitis | xylitol dehydrogenase | 53 |
| XYL3 from Pichia stipitis | xylulokinase | 54 |
| XYN1 from Pichia stipitis | endo-1,4-beta-xylanase | 55 |
| TKL2 from Pichia stipitis | transketolase | 92 |
| TKT1 from Pichia stipitis | transketolase | 93 |
| TAL1 from Pichia stipitis | transaldolase | 94 |

TABLE 3
Summary of the terminator sequences used in this study

| Description | SEQ ID NO: Nucleic acid |
|---|---|
| ACB2 from Pichia stipitis | 56 |
| ALD1 from Saccharomyces cerevisiae | 57 |
| BGL1 from Pichia stipitis | 58 |
| BGL2 from Pichia stipitis | 59 |
| BGL3 from Pichia stipitis | 60 |
| BGL4 from Pichia stipitis | 61 |
| BGL5 from Pichia stipitis | 62 |
| BGL6 from Pichia stipitis | 63 |
| BGL7 from Pichia stipitis | 64 |
| EGC1 from Pichia stipitis | 65 |
| EGC2 from Pichia stipitis | 66 |
| EGC3 from Pichia stipitis | 67 |
| FAS2 from Pichia stipitis | 68 |
| HGT1 from Pichia stipitis | 69 |
| HGT2 from Pichia stipitis | 70 |
| HXT2.1 from Pichia stipitis | 71 |
| HXT2.2 from Pichia stipitis | 72 |
| HXT2.3 from Pichia stipitis | 73 |
| HXT2.4 from Pichia stipitis | 74 |
| HXT2.5 from Pichia stipitis | 75 |
| HXT2.6 from Pichia stipitis | 76 |
| HXT4 from Pichia stipitis | 77 |
| SUT1 from Pichia stipitis | 78 |
| SUT2 from Pichia stipitis | 79 |
| SUT3 from Pichia stipitis | 80 |
| SUT4 from Pichia stipitis | 81 |
| TDH3 from Pichia stipitis | 82 and 83 |
| TDH3 from Saccharomyces cerevisiae | 84 and 85 |
| TEF2 from Saccharomyces cerevisiae | 86 |
| TPI1 from Saccharomyces cerevisiae | 87 |
| XUT1 from Pichia stipitis | 88 |
| XUT3 from Pichia stipitis | 89 |
| XYN1 from Pichia stipitis | 90 |
| ZWF1 from Pichia stipitis | 91 |

TABLE 4
Pichia stipitis strains

| | | Source or |
|---|---|---|
| Strain | Description | reference |
| P. stipitis Y-7124 | Wild-type strain | NRRL Y-7124 |
| P. stipitis Y-7124.1.136 | XUT1 | This study |
| P. stipitis Y-7124.1.144 | XUT1 + sSUT4 | This study |
| P. stipitis Y-7124.1.155 | XUT1 + sSUT4 + HXT4 + sZmADH1 | This study |
| P. stipitis Y-7124.1.158 | XUT1 + XYL123 | This study |
| P. stipitis Y-7124.1.159 | XUT1 + XYL123 | This study |
| P. stipitis Y-7124.1.160 | XUT1 + XYL123 | This study |
| P. stipitis Y-7124.1.161 | XUT1 + XYL123 | This study |
| P. stipitis Y-7124.1.162 | XUT1 + XYL123 | This study |
| P. stipitis Y-7124.1.163 | XUT1 + XYL123 | This study |
| P. stipitis Y-7124.1.164 | XUT1 + sSUT4 + sXmADH1 | |
| P. stipitis Y-7124.1.165 | XUT1 + sSUT4 + sXmADH1 | This study |
| P. stipitis Y-7124.1.166 | XUT1 + sSUT4 + sXmADH1 | This study |
| P. stipitis Y-7124.1.167 | XUT1 + sSUT4 + sXmADH1 | This study |
| P. stipitis Y-7124.1.168 | XUT1 + sSUT4 + sXmADH1 | This study |
| P. stipitis Y-7124.1.169 | XUT1 + sSUT4 + sXmADH1 | This study |
| P. stipitis Y-7124.1.170 | XUT1 + sSUT4 + HXT4 | This study |
| P. stipitis Y-7124.1.171 | XUT1 + sSUT4 + HXT4 | This study |
| P. stipitis Y-7124.1.172 | XUT1 + sSUT4 + HXT4 | This study |
| P. stipitis Y-7124.1.173 | XUT1 + sSUT4 + HXT4 | This study |
| P. stipitis Y-7124.1.174 | XUT1 + sSUT4 + HXT4 | This study |
| P. stipitis Y-7124.1.175 | XUT1 + sSUT4 + HXT4 | This study |
| P. stipitis Y-7124.1.176 | XUT1 + sSUT4 + XUT3 | This study |
| P. stipitis Y-7124.1.177 | XUT1 + sSUT4 + XUT3 | This study |
| P. stipitis Y-7124.1.178 | XUT1 + sSUT4 + XUT3 | This study |
| P. stipitis Y-7124.1.179 | XUT1 + sSUT4 + XUT3 | This study |
| P. stipitis Y-7124.1.180 | XUT1 + sSUT4 + XUT3 | This study |
| P. stipitis Y-7124.1.181 | XUT1 + sSUT4 + XUT3 | This study |
| P. stipitis Y-7124.1.182 | XUT1 + XYL123 + sSUT4 | This study |
| P. stipitis Y-7124.1.183 | XUT1 + XYL123 + sSUT4 | This study |
| P. stipitis Y-7124.1.184 | XUT1 + XYL123 + sSUT4 | This study |
| P. stipitis Y-7124.1.185 | XUT1 + XYL123 + sSUT4 | This study |
| P. stipitis Y-7124.1.186 | XUT1 + XYL123 + sSUT4 | This study |
| P. stipitis Y-7124.1.187 | XUT1 + XYL123 + sSUT4 | This study |
| P. stipitis Y-7124.2.344 | XYL12 + sSUT4 | This study |
| P. stipitis Y-7124.2.345 | sSUT4 | This study |
| P. stipitis Y-7124.2.346 | sSUT4 | This study |
| P. stipitis Y-7124.2.347 | sSUT4 | This study |
| P. stipitis Y-7124.2.348 | sSUT4 | This study |
| P. stipitis Y-7124.2.349 | sSUT4 | This study |
| P. stipitis Y-7124.2.350 | sSUT4 | This study |
| P. stipitis Y-7124.2.351 | sSUT4 | This study |
| P. stipitis Y-7124.2.352 | sSUT4 | This study |
| P. stipitis Y-7124.2.353 | sSUT4 | This study |
| P. stipitis Y-7124.2.354 | sSUT4 | This study |
| P. stipitis Y-7124.2.405 | XYL12 + sSUT4 + sZmADH1 | This study |
| P. stipitis Y-7124.2.406 | XYL12 + sSUT4 + sZmADH1 | This study |
| P. stipitis Y-7124.2.407 | XYL12 + sSUT4 + sZmADH1 | This study |
| P. stipitis Y-7124.2.408 | XYL12 + sSUT4 + sZmADH1 | This study |
| P. stipitis Y-7124.2.409 | XYL12 + sSUT4 + sZmADH1 | This study |
| P. stipitis Y-7124.2.415 | XYL123 + sSUT4 | This study |
| P. stipitis Y-7124.2.416 | XYL123 + sSUT4 | This study |
| P. stipitis Y-7124.2.417 | XYL123 + sSUT4 | This study |
| P. stipitis Y-7124.2.418 | XYL123 + sSUT4 | This study |
| P. stipitis Y-7124.2.419 | XYL123 + sSUT4 | This study |
| P. stipitis Y-7124.2.446 | sSUT4 + HXT4 | This study |
| P. stipitis Y-7124.2.447 | sSUT4 + HXT4 | This study |
| P. stipitis Y-7124.2.448 | sSUT4 + HXT4 | This study |
| P. stipitis Y-7124.2.449 | XYL12 + sSUT4 + sZmADH1 + HXT4 | This study |
| P. stipitis Y-7124.2.450 | XYL12 + sSUT4 + sZmADH1 + HXT4 | This study |
| P. stipitis Y-7124.2.451 | XYL12 + sSUT4 + sZmADH1 + HXT4 | This study |
| P. stipitis Y-7124.2.452 | XYL12 + sSUT4 + sZmADH1 + HXT4 | This study |
| P. stipitis Y-7124.2.453 | XYL12 + sSUT4 + sZmADH1 + HXT4 | This study |
| P. stipitis Y-7124.2.454 | XYL12 + sSUT4 + sZmADH1 + HXT4 | This study |
| P. stipitis Y-7124.2.455 | XYL12 + sSUT4 + sZmADH1 + XUT3 | This study |
| P. stipitis Y-7124.2.456 | XYL12 + sSUT4 + sZmADH1 + XUT3 | This study |
| P. stipitis Y-7124.2.457 | XYL12 + sSUT4 + sZmADH1 + XUT3 | This study |
| P. stipitis Y-7124.2.458 | XYL12 + sSUT4 + sZmADH1 + XUT3 | This study |
| P. stipitis Y-7124.2.459 | XYL12 + sSUT4 + sZmADH1 + XUT3 | This study |
| P. stipitis Y-7124.2.460 | XYL12 + sSUT4 + sZmADH1 + XUT3 | This study |
| P. stipitis Y-7124.2.462 | sSUT4 + XYL123 + HXT4 + sZmADH1 | This study |
| P. stipitis Y-7124.2.465 | XUT3 | This study |
| P. stipitis Y-7124.2.466 | XUT3 | This study |
| P. stipitis Y-7124.2.467 | XUT3 | This study |
| P. stipitis Y-7124.2.468 | XUT3 | This study |
| P. stipitis Y-7124.2.469 | HXT4 + sZmADH1 | This study |
| P. stipitis Y-7124.2.470 | HXT4 + sZmADH1 | This study |
| P. stipitis Y-7124.2.471 | HXT4 | This study |
| P. stipitis Y-7124.2.472 | HXT4 | This study |
| P. stipitis Y-7124.2.474 | XYL12 + HXT4 | This study |
| P. stipitis Y-7124.2.477 | sSUT4 + sZmADH | This study |
| P. stipitis Y-7124.2.478 | sSUT4 + sZmADH | This study |
| P. stipitis Y-7124.2.479 | sSUT4 + sZmADH | This study |
| P. stipitis Y-7124.2.480 | sSUT4 + sZmADH | This study |
| P. stipitis Y-7124.2.481 | sSUT4 + sZmADH | This study |
| P. stipitis Y-7124.2.482 | sSUT4 + XYL123 + XUT1 | This study |
| P. stipitis Y-7124.2.483 | sSUT4 + XYL123 + XUT1 | This study |
| P. stipitis Y-7124.2.484 | sSUT4 + XYL123 + XUT1 | This study |
| P. stipitis Y-7124.2.485 | sSUT4 + XYL123 + XUT1 | This study |
| P. stipitis Y-7124.2.486 | sSUT4 + XYL123 + XUT1 | This study |
| P. stipitis FPL-Y-UC7 | ura3 | NRRL Y-21448 |
| P. stipitis Y-UC7.1.101 | HXT2.4 | This study |
| P. stipitis Y-UC7.1.102 | BGL5 cluster (HXT2.4, EGC2, BGL5) | This study |
| P. stipitis Y-7124.2.535 | 2[sSUT4] + XYL1 + XYL2 + XYL3 | This study |
| P. stipitis Y-7124.2.538 | 2[sSUT4] + XYL1 + XYL2 + XYL3 | This study |
| P. stipitis Y-7124.2.541 | sSUT4 + XYL1 + XYL2 + TAL1 + TKT1 | This study |
| P. stipitis Y-7124.2.557 | 7124.2.535-539 × 6054.2.356-359 | This study |
| P. stipitis Y-7124.2.558 | 7124.2.546-549 × 6054.2.356-359 | This study |

TABLE 5
Saccharomyces cerevisiae strains

| Strain | Description | Source or reference |
|---|---|---|
| S. cerevisiae | MATa leu2-3112 trp1-289 | Entian K, Kotter P, 2007, |
| CEN. PK. | MAL2-8c SUC2 | 25 Yeast Genetic Strain and |
| 111-27B | | Plasmid Collections. |
| | | In: Methods in Microbiology; |
| | | Yeast Gene Analysis- |
| | | Second Edition, Vol. |
| | | Volume 36 (Ian Stansfield |
| | | and Michael J R Stark ed), |
| | | pp 629-666. Academic Press. |
| S. cerevisiae | CEN. PK. 111-27B | This study |
| SSN7 | (pRS424 and pRS425) | |
| S. cerevisiae | CEN. PK. 111-27B | This study |
| SSN17 | (pSN260 and pSN259) | |
| S. cerevisiae | CEN. PK. 111-27B | This study |
| SSN18 | (pSN261 and pSN259) | |
| S. cerevisiae | CEN. PK. 111-27B | This study |
| SSN21 | (pSN264 and pSN259) | |
| S. cerevisiae | CEN. PK. 111-27B | This study |
| SSN23 | (pSN266 and pSN259) | |

TABLE 6
Plasmids

| Plasmid | Description | Source or reference |
|---|---|---|
| pRS424 | TRP1, 2μ origin | Sikorski & Hieter, 1989, |
| | | Genetics 122: 19-27 |
| pRS425 | LEU2, 2μ origin | Sikorski & Hieter, 1989, |
| | | Genetics 122: 19-27 |
| pRS315 | LEU2, Centromere | Sikorski & Hieter, 1989, |
| | | Genetics 122: 19-27 |
| pSN259 | TRP1, 2μ origin ScTDH3_{P}-PsBGL5-ScTDH3_{T} | This study |
| pSN260 | LEU2, Centromere ScTDH3_{P}-PsHXT2.4-ScTDH3_{T} | This study |
| pSN261 | LEU2, Centromere ScTDH3_{P}-PsHXT2.2- | This study |
| | ScTDH3_{T} | |
| pSN264 | LEU2, Centromere ScTDH3_{P}-PsHXT2.5- | This study |
| | ScTDH3_{T} | |
| pSN266 | LEU2, Centromere ScTDH3_{P}-PsHXT2.6- | This study |
| | ScTDH3_{T} | |
| pSN321 | XUT1 in pSDM11 | This study |
| pSN207 | HXT2.4 in pJYB11 | This study |
| pSN212 | BGL5, EGC2, HXT2.4 in pJYB11 | This study |
| pJYB11 | PsURA3 in pBluescript KS- | |
| pJML545 | cre recombinase expression vector | Laplaza, et. al, 2006, |
| | | Enzyme & Micro Tech, |
| | | 38: 741-747 |
| pSDM11 | synNATI in pBluescript KS- | This study |
| pSDM20 | PsZWF1_{P}-PsXYL3-PsZWF1_{T}-PsTDH3_{P}- | This study |
| | PsXYL2-PsTDH3_{T}-PsFAS2_{P}- | |
| | PsXYL1_PsFAS2_{T} in pSDM11 | |
| pSDM21 | PsTDH3_{P}-sZmADH1-PsTDH3_{T} in | This study |
| | pSDM11 | |
| pSDM22 | PsTDH3_{P}-PsHXT4 in pSDM11 | This study |
| pSDM24 | PsTDH3_{P}-PsXYL2-PsTDH3_{T}-PsFAS2_{P}- | This study |
| | PsXYL1-PsFAS2_{T}-PsTDH3_{P}-PsHXT4 in | |
| | pSDM11 | |
| pSDM25 | PsTDH3_{P}-sZmADH1-PsTDH3_{T}-PsTDH3_{P}- | This study |
| | PsHXT4 in pSDM11 | |
| pSDM29 | PsTDH3_{P}-sSUT4-PsSUT4_{T} in pSDM11 | This study |
| pSDM30 | PsTDH3_{P}-sSUT4-PsSUT4_{T} PsTDH3_{P}- | This study |
| | sZmADH1-PsTDH3_{T} in pSDM11 | |
| pSDM31 | PsTKT1_{P}-XUT3-PsXUT3_{T} in pSDM11 | This study |
| pSDM32 | PsTDH3_{P}-PsXYL2-PsTDH3_{T}-PsFAS2_{P}- | This study |
| | PsXYL1-PsFAS2_{T}-PsTDH3_{P}-sSUT4- | |
| | PsSUT4_{T} in pSDM11 | |
| pMA300 | PsTAL1_{P}-PsTAL1-PsTAL1_{T}-PsTKT1_{P}- | This study |
| | PsTKT1-PsTKT1_{T} in pSDM11 | |

### III. Conversion of Cellobiose to Ethanol

It has been discovered the cellobiose utilization and conversion to ethanol in yeast can be greatly improved by expression of one or more cellobiose transporter and one or more beta-glucosidase in the yeast.

Exemplary cellobiose transporters can include, but are not limited to, e.g., the HXT transporters from Pichia stipitis, e.g., HXT2.1, HXT2.2, HXT2.3, HXT2.4, HXT2.5, or HXT2.6. In some embodiments, the cellobiose transporter is substantially identical to any of SEQ ID NO:s 38, 39, 40, 41, 42, 43, or 44. In some embodiments, the cellobiose transporter is recombinantly expressed from an introduced expression cassette comprising a promoter operably linked to a polynucleotide encoding the cellobiose transporter. The promoter can be a native (i.e., native to the transporter) promoter. Alternatively, the promoter can be a heterologous promoter, e.g., not a promoter found in association in nature with the cellobiose transporter gene. Exemplary promoters include, but are not limited to, any of those described in Table 1. Similarly, native or heterologous terminator sequences can be used. Exemplary terminator sequences include, but are not limited to those in Table 3.

Exemplary beta-glucosidases can include, but are not limited to, e.g., a beta-glucosidase from Pichia stipitis, e.g., BGL1, BGL2, BGL3, BGL4, BGL5, BGL6, or BGL7. In some embodiments, the beta-glucosidase is substantially identical to any of SEQ ID NO:s 26, 27, 28, 29, 30, 31, or 32. In some embodiments, the beta-glucosidase is recombinantly expressed from an introduced expression cassette comprising a promoter operably linked to a polynucleotide encoding the beta-glucosidase. The promoter can be a native (native to the beta-glucosidase) promoter. Alternatively, the promoter can be a heterologous promoter, e.g., not a promoter found in association in nature with the beta-glucosidase gene. Exemplary promoters include, but are not limited to, any of those described in Table 1. Similarly, native or heterologous terminator sequences can be used. Exemplary terminator sequences include, but are not limited to those in Table 3.

In some embodiments, the yeast is of the genus Saccharomyces (e.g., S. cerevisiae) or Pichia (e.g., P. stipitis).

In some embodiments, the yeast utilizes cellobiose at a rate of at least 0.10, 0.15, 0.17, 0.19, 0.22, or 0.25 g/l per hour.

In some embodiments, the yeast also converts a C5 sugar (e.g., xylose) into ethanol. For example, the yeast can also be engineered with a xylose transporter as described herein, in combination with one, two, or all of a xylose reductase, a xylitol dehydrogenase, and/or a xylulokinase; and optionally can further express a transketolase and/or a transaldolase as otherwise described herein.

Accordingly, the invention also provides for conversion of cellobiose in a mixture with a yeast as described above. Any source of cellobiose is contemplated for use with the yeast of the invention. The conversion process can be performed in batch-wise or as a continuous process, and can be performed, for example, in a bioreactor.

### IV. Conversion of Xylose to Ethanol

It has been discovered that xylose utilization and conversion to ethanol in yeast can be greatly improved by expression of one or more xylose transporters and one or more of a xylose reductase, a xylitol dehydrogenase, and/or a xylulokinase in the yeast, as shown in the Examples. Surprisingly, this increases xylose utilization in Pichia stipitis, which naturally expresses some or all of these genes.

Exemplary xylose transporters can include, but are not limited to, the SUT and XUT transporters from Pichia stipitis, e.g., SUT 1, SUT 2, SUT3, SUT4, XUT1 or XUT3. The SUT1-4 transporters are also glucose transporters. In some embodiments, the xylose transporter is substantially identical to any of SEQ ID NOS: 46, 47, 48, 49, 50, or 51. In some embodiments, the xylose transporter is recombinantly expressed from an introduced expression cassette comprising a promoter operably linked to a polynucleotide encoding the xylose transporter. The promoter can be a native promoter (i.e., the promoter that naturally regulates expression of the polynucleotide encoding the transporter in the yeast cell). Alternatively, the promoter can be a heterologous promoter, e.g., not a promoter found in association in nature with the xylose transporter gene. Exemplary promoters include, but are not limited to, any of those described in Table 1. Similarly, native or heterologous terminator sequences can be used. Exemplary terminator sequences include, but are not limited to those in Table 3.

Exemplary xylose reductases include, but are not limited to, the XYL1 reductases from Pichia stipitis. In one embodiment, the xylose reductase is substantially identical to SEQ ID NO: 52. Exemplary xylitol dehydrogenases include, but are not limited to, the XYL2 dehydrogenase from Pichia stipitis. In one embodiment, the xylitol dehydrogenase is substantially identical to SEQ ID NO: 53. Exemplary xylulokinases include, but are not limited to, the XYL3 xylulokinase from Pichia stipitis. In one embodiment, the xylulokinase is substantially identical to SEQ ID NO: 54. In some embodiments, the xylose reductase, xylitol dehydrogenase, or xylulokinase is recombinantly expressed from an introduced expression cassette comprising a promoter operably linked to a polynucleotide encoding the xylose reductase, xylitol dehydrogenase, or xylulokinase. The promoter can be a native promoter (i.e., the promoter that naturally regulates expression of the polynucleotide in the yeast cell). Alternatively, the promoter can be a heterologous promoter, e.g., not a promoter found in association in nature with the xylose reductase, xylitol dehydrogenase, or xylulokinase gene. Exemplary promoters include, but are not limited to, any of those described in Table 1. Similarly, native or heterologous terminator sequences can be used. Exemplary terminator sequences include, but are not limited to those in Table 3.

In some embodiments, the yeast further comprises a transketolase and/or a transaldolase. Exemplary transketolases include, but are not limited to, TKL2 and TKT1 from Pichia stipitis. In some embodiments, the transketolase is substantially identical to SEQ ID NOS: 92 or 93. Exemplary transaldolases include, but are not limited to, TAL1 from Pichia stipitis. In one embodiment, the transketolase is substantially identical to SEQ ID NO: 94. Surprisingly, expression of a P. stipitis transketolase and/or a P. stipitis transaldolase increases xylose utilization in P. stipitis, which naturally expresses some or all these genes, as shown in the Examples.

In some embodiments, the yeast is of the genus Saccharomyces (e.g., S. cerevisiae) or Pichia (e.g., P. stipitis).

In some embodiments, the yeast utilizes xylose at a rate of at least 0.5, 0.7, 1.0, 1.1, 1.2, 1.3, 1.5, 1.7, 1.8, 1.9, 2.0, 2.2, 2.3, 2.5, 2.6, 2.7, 2.9, 3.0, 3.2, 3.3, 3.4, 3.5, or 4.0 g/l per hour or higher.

In some embodiments, the yeast comprises two or more xylose transporters. For example, the yeast can be engineered with a first expression cassette comprising a first xylose transporter, and a second expression cassette comprising a second xylose transporter. In some embodiments, the first and second xylose transporters are the same or different. For example, in one embodiment, the first and second xylose transporters are SUT4. In other embodiments, the first and second xylose transporters are substantially identical to SEQ ID NO:49. The expression of two xylose transporters improves the utilization of xylose, as described in the Examples.

In other embodiments, the yeast comprises, or further comprises, two or more of each of a xylose reductase, xylitol dehydrogenase, or a xylulokinase, as described above. The expression of two or more xylose reductases, xylitol dehydrogenases, and/or xylulokinases improves the utilization of xylose, as described in the Examples.

In some embodiments, the yeast also converts a C6 sugar (e.g., glucose) into ethanol. For example, the yeast can be engineered with one or more of a cellobiose transporter, a beta-glucosidase, and/or an endo-1,4-beta-glucanase, as described herein.

Accordingly, the invention also provides for conversion of xylose in a mixture with a yeast as described above. Any source of xylose is contemplated for use with the yeast of the invention. The conversion process can be performed in batch-wise or as a continuous process.

### V. Production of Sequences and Yeast Strains

The nucleic acid sequences recombinantly expressed in the improved yeast cells of the present invention can be naturally derived or synthetically produced. The nucleic acid and amino acid sequences of the different transporters and sugar metabolizing enzymes are known in the art and described herein. When designing nucleic acid sequences for expression in P. stipitis or S. cerevisiae, it is to be considered that the codon CUG encodes for a serine residue in P. stipitis and for a leucine residue in S. cerevisiae. See, e.g., U.S. Patent Publication No. 2006/0088911.

The genes can consist of DNA native to the host organism or synthetic that code for various metabolic activities. These can include but are not limited to sugar transporters, oxidoreductases, transketolases, transaldolases, pyruvate decarboxylases, aldose reductase, xylitol dehydrogenase, alcohol dehydrogenases, D-xylulokinase, pyruvate decarboxylase, beta-glucosidase, endo-1,4-β-D-glucanase and various combinations of same along with native or synthetic genes for resistance to nourseothricin, zeocin, hygromycin or other antibiotic inhibitors flanked by sequences to promote their excision.

The genes and promoters for altering their native expression are identified through Southern hybridization, quantitative PCR (qPCR), quantitative expressed sequence tag (EST) sequencing, expression array analysis, or other methods to measure the abundance of transcripts. Cells are cultivated under varying conditions such as with various carbon or nitrogen sources, under different aeration conditions, at various temperatures or pH, in the presence of various effector molecules such as inducers, inhibitors or toxins or in the presence of stressors such as high sugar or product concentrations. The resulting transcript expression levels are correlated with the rates of product formation to determine which transcripts are expressed at high levels and which are present at relatively low levels under conditions favoring product formation. These data in turn are correlated with information about enzymes or metabolic activities known to be essential for product formation from the substrate or under the conditions desired for maximal performance.

Introduction of the recombinant nucleic acid sequences into a yeast cell can be accomplished by any suitable means. For example, the recombinant expression cassette can be incorporated intrachromosomally or extrachromosomally. The expression cassettes can be introduced sequentially, e.g., using a Cre-loxP technique e.g., facilitating removal using cre recombinase following single or repeated transformations and excisions of a selectable marker (U.S. Pat. No. 7,501,275 B2; and Laplaza, et. al, 2006, Enzyme & Microbial Tech, 38:741-747). Two or more expression cassettes also can be concurrently introduced, e.g., using so-called recombineering techniques that utilize homologous recombination. It is envisioned that one could obtain increased expression of the nucleic acid constructs of the invention using an extrachromosomal genetic element, by integrating additional copies, e.g., of either native or heterologous genes, by increasing promoter strength, or by increasing the efficiency of translation through codon optimization, all methods known to one of skill in the art.

As noted in the examples, mating of two or more separately transformed and genetically different strains of yeast and subsequent selection of the resulting hybrid progeny can result in additional improvement in C5 and/or C6 sugar utilization and generation of ethanol. In some embodiments, one of the mated strains has the CBS 6054 genetic background and a second strain has the NRRL Y-7124 genetic background.

The promoters for genes expressed at high levels under the desired conditions for maximal performance and product formation were then used to drive expression of transcripts for genes present at relatively low levels. The resulting transformants were assessed to determine whether increased expression of the targeted gene or combination of genes increases product formation. Relative product formation rates were determined by cultivation of native, parental or other wild-type or engineered strains in parallel with or sequentially to the cultivation of genetically altered strains.

In another embodiment, promoters for genes expressed at levels deemed to be excessive for optimal product formation can be reduced in expression by substituting weaker promoters or by altering the coding sequence to render lower protein activity.

The constructs of the invention comprise a coding sequence operably connected to a promoter. Preferably, the promoter is a constitutive promoter functional in yeast, or an inducible promoter that is induced under conditions favorable to uptake of sugars or to permit fermentation. Inducible promoters may include, for example, a promoter that is enhanced in response to particular sugars, or in response to oxygen limited conditions, such as the FAS2 promoter used in the examples. Examples of other suitable promoters include promoters associated with genes encoding P. stipitis proteins which are induced in response to xylose under oxygen limiting conditions, including, but not limited to, myo-inositol 2-dehydrogenase (MOR1), aminotransferase (YOD1), guanine deaminase (GAH1). These proteins correspond to protein identification numbers 64256, 35479, and 36448 on the Joint Genome Institute Pichia stipitis web site: genome.jgi-psf.org/Picst3/Picst3.home.html.

Medium constituents and conditions can range from minimal defined nutrients to complex formulations having many different carbon and nitrogen sources including but not limited to acid and enzymatic hydrolysates of pretreated lignocellulosic substrates.

Oxygen limiting conditions include conditions that favor fermentation. Such conditions, which are neither strictly anaerobic nor fully aerobic, can be achieved, for example, by growing liquid cultures with reduced aeration, i.e., by reducing shaking, by increasing the ratio of the culture volume to flask volume, by inoculating a culture medium with a number of yeast effective to provide a sufficiently concentrated initial culture to reduce oxygen availability, e.g., to provide an initial cell density of 1.0 g/l dry wt of cells. Suitable minimal media for growth of the yeast cells is described, e.g., in Verduyn, et al., (1992) Yeast 8:501-17 and herein.

Preferably, the yeast strain is able to grow under conditions similar to those found in industrial sources of xylose. The method of the present invention would be most economical when the xylose-containing material can be inoculated with the mutant yeast without excessive manipulation. By way of example, the pulping industry generates large amounts of cellulosic waste. Saccharification of the cellulose by acid hydrolysis yields hexoses and pentoses that can be used in fermentation reactions. However, the hydrolysate or sulfite liquor contains high concentrations of sulfite and phenolic inhibitors naturally present in the wood which inhibit or prevent the growth of most organisms. Serially subculturing yeast selects for strains that are better able to grow in the presence of sulfite or phenolic inhibitors.

The yeast cells of the invention find use in fermenting xylose in a xylose-containing material to produce ethanol using the yeast of the invention as a biocatalyst. For example, the yeast cells of the invention find use in fermenting xylose in a xylose-containing material to produce xylitol using the yeast of the invention as a biocatalyst. In this embodiment, the yeast preferably has reduced xylitol dehydrogenase activity such that xylitol is accumulated. Preferably, the yeast is recovered after the xylose in the medium is fermented to ethanol and used in subsequent fermentations.

It is expected that yeast strains of the present invention may be further manipulated to achieve other desirable characteristics, or even higher specific ethanol yields. For example, selection of mutant yeast strains by serially cultivating the mutant yeast strains of the present invention on medium containing hydrolysate may result in improved yeast with enhanced fermentation rates.

The yeast cells of the invention may be selected for their ability to produce high ethanol yields in a relatively short period of time (e.g., under about 72 hours, for example, within about 40, 45, 55, 60, 65, 70 hours). The yeast cells of the invention can produce ethanol with a yield of at least about 0.3 g ethanol/g sugar consumed (e.g., at least about 0.4, 0.5, 0.6, 0.7, 0.8 g ethanol/g sugar consumed); culture media with ethanol concentrations of at least about 40 g ethanol/l (e.g., at least about 45, 50, 55, 60, 65, 70, 75 g ethanol/l) and can have an ethanol production rate of at least about 0.5 g/l·h (e.g., at least about 0.5, 0.6, 0.7, 0.8, 0.9, 1.0 g/l·h). The yeast cells may also be selected for their tolerance (i.e., the ability to remain viable) in culture conditions with high concentrations of ethanol, e.g., with ethanol concentrations of at least about 40 g ethanol/l (e.g., at least about 45, 50, 55, 60, 65, 70, 75, 80, 85 g ethanol/l). In some embodiments, the yeast cells of the invention are tolerant to culture media containing concentrations of at least about 5% ethanol, for example, at least about 6%, 7%, 8% or more, ethanol.

Acetate and acetic acid are released from the lignocellulosic substrate by hydrolysis or are byproducts of fermentation. High concentrations of acetic acid can inhibit fermentation, and in some instances, growth. Accordingly, in some embodiments, the yeast cells of the invention are selected for their tolerance to culture conditions with high concentrations of acetic acid, and correspondingly relatively acid pH. Most yeast cells are tolerant to culture fluid concentrations of acetic acid in the range of 0-3 g/L. Yeast cells that efficiently utilize substrate may need to be tolerant to higher concentrations of acetic acid to maintain commercially viable levels of fermentation and/or growth. Accordingly, in some embodiments, yeast cells that are tolerant to culture media containing concentrations of acetic acid of at least about 3 g/L and as high as 15 g/L, for example, in the range of about 5-10 g/L, for example, at least about 4 g/L, 5 g/L, 6 g/L, 7 g/L, 8 g/L, 9 g/L, 10 g/L, 11 g/L, 12 g/L, 13 g/L, 14 g/L, 15 g/L, or higher, are selected. Such yeast cells are tolerant to more acidic pH, for example, a pH less than about 6, for example, in the range of pH 4-6, for example, a pH of about 6.0, 5.5, 5.0, 4.5, 4.0, or less.

In some embodiments, the yeast cells are selected for their ability to convert sugars to ethanol in the presence of acetic acid. For example, in certain embodiments, the yeast cells can convert sugars to ethanol in the presence of concentrations of acetic acid in the range of about 0.1 g/L to about 5 g/L, for example, at least about 0.2 g/L, 0.3 g/L, 0.4 g/L, 0.5 g/L, 0.6 g/L, 0.7 g/L, 0.8 g/L, 0.9 g/L, 1.0 g/L, 1.1 g/L, 1.2 g/L, 1.3 g/L, 1.4 g/L, 1.5 g/L, 1.6 g/L, 1.7 g/L, 1.8 g/L, 1.9 g/L, 2.0 g/L, 2.1 g/L, 2.2 g/L, 2.3 g/L, 2.4 g/L, 2.5 g/l, 2.6 g/L, 2.7 g/l, 2.8 g/L, 2.9 g/L, 3.0 g/L, 3.5 g/l, 4.0 g/L, 4.5 g/L and 5.0 g/L. In other embodiments, the yeast cells can convert sugars to ethanol in the presence of concentrations of acetic acid in the range of about 0.05% to about 0.5%, for example, at least about 0.075%, 0.085%, 0.10%, 0.11%, 0.12%, 0.13%, 0.14%, 0.15%, 0.16%, 0.17%, 0.18%, 0.19%, 0.20%, 0.25%, 0.30%, 0.35%, 0.40%, 0.45%, and 0.50%. In other embodiments, the yeast cells can convert sugars to ethanol in the presence of concentrations of acetic acid in the range of about 0.50% to about 5.0%, for example, at least about 0.60%, 0.70%, 0.80%, 0.90%, 1.0%, 1.5%, 2.0%, 2.5%, 3.0%, 3.5%, 4.0%, 4.5%, and 5.0%.

In some embodiments, the yeast cells are selected to convert both C6 and C5 sugars to ethanol in presence of acetic acid. In one embodiment, the yeast cells are selected to convert both glucose and xylose to ethanol in presence of acetice acid. In another embodiment, the yeast cells are selected to convert both cellobiose and xylose to ethanol in presence of acetice acid.

In certain embodiments, the yeast cells are selected to have increased rates of Xylose fermentation. In other embodiments, the yeast cells are selected to have increased rates of acetic acid removal.

In other embodiments, the yeast cells are adapted to grow in increasing concentrations of acetic acid. For example, in certain embodiment, the yeast cells are adapted to grow in concentrations of acetic acid up between about 0.1% to 0.5%.

Yeast cells cultured in medium containing high concentrations of sugar may be subject to relatively higher osmotic pressures. Growth of Pichia stipitis begins to slow down at sugar concentrations in excess of about 80 g/l. Accordingly, in some embodiments, yeast cells that are tolerant to culture media containing concentrations of sugar of at least about 80 g/L and as high as 200 g/L, for example, in the range of about 140-200 g/L or 140-160 g/L, for example, at least about 90 g/L, 100 g/L, 110 g/L, 120 g/L, 130 g/L, 140 g/L, 150 g/L, 160 g/L, 170 g/L, 180 g/L, 190 g/L, 200 g/L, or higher, are selected.

The present yeast cells find use in commercial scale fermentation processes, for example, in bioreactors containing culture media in volumes of at least 100L, for example, at least about 500L, 1000L, 5000L, 10,000L, 20,000L, 50,000L, 100,000L, or more.

In accordance with the present invention, there may be employed conventional molecular biology, microbiology, and biochemical techniques within the skill of the art. Such techniques are explained fully in the literature. The invention will be further described in the following examples, which do not limit the scope of the invention described in the claims. The methods and materials described herein can be incorporated into existing biofuels operations, or the methods and materials described herein can be included in designing new biofuels operations.

### EXAMPLES

The following examples are offered to illustrate, but not to limit the claimed invention.

### Example 1

### Production of Yeast Cells that Produce High Levels of Ethanol

A modified defined minimal medium was used containing trace metal elements and vitamins, which is based on that described by Verduyn et al. (10) It had the following composition: 1.9 g urea 1⁻¹; 5.2 g peptone 1⁻¹; 14.4 g KH₂PO₄ 1⁻¹; 0.5 g MgSO₄.7H₂O 1⁻¹; 4 ml trace element solution 1⁻¹; 2 ml vitamin solution 1⁻¹; and 0.05 ml antifoam 289 (Sigma A-8436) 1⁻¹. Glucose and xylose concentrations were varied in some experiments.

A synthetic NAT1 gene was fused to the P. stipitis ACB2 promoter and terminator, and LoxP sites flanked the entire cassette, facilitating removal using cre recombinase following single or repeated transformations and excisions of the selectable marker (Jose M. Laplaza and T. W. Jeffries, U.S. Pat. No. 7,501,275 B2; Laplaza, et. al, 2006, Enzyme & Microbial Tech, 38:741-747) (7). The NAT1 gene could be removed by transforming the transformants with approximately 10 μg of pJML545, which encodes a cre recombinase that facilitates the removal of the LoxP flanked NAT1 marker.

The LiAc protocol of Gietz & Woods (2) was routinely used for cell transformation.

The amino acid sequence of the Streptomyces noursei Nat1p was used to generate the NAT1 gene, which was optimized for codon usage found in Pichia stipitis and Saccharomyces cerevisiae and synthesized by DNA2.0 Inc. (Menlo Park, Calif. 94025). The synthetic NAT1 gene was fused to the P. stipitis ACB2 promoter and terminator, and LoxP sites flanked the entire cassette, facilitating removal using cre recombinase (7). This final product was cloned into pBluescript KS-, generating pSDM11.

pSN321 was constructed to contain the promoter, coding sequence, and terminator for the P. stipitis XUT1 gene. Approximately 100 μg of plasmid was linearized using the restriction enzymes SpeI and ApaI, ethanol precipitated, resuspended in water, creating a fragment that could be directly inserted into the P. stipitis genome. The digested construct was then transformed into NRRL Y-7124 using a LiAc protocol (2), thereby creating 7124.1.136, and into 7124.2.415 creating 7124.2.482, 7124.2.483, 7124.2.484, 7124.2.485, and 7124.2.486.

Transformants were selected via growth on YPD plates containing 50 μg/ml nourseothricin and dextrose (2%). Colonies were grown overnight in YPD+50 μg/ml nourseothricin liquid medium, genomic DNA was prepped and evaluated by PCR to confirm integration of the fragment.

The NAT1 gene was removed by transforming the transformants with pJML545 (7). Transformants were selected on YPD plates containing 50 μg/ml zeocin and dextrose (2%). Colonies were patched onto YPD and YPD nourseothricin plates to confirm excision of the NAT1 marker.

Fermentation of 7124.1.136. Cultures were started by inoculating a swath of colonies into 25 ml YPX (2% xylose) and grown overnight. The following morning, triplicate flasks were inoculated to a starting OD600 of 9.0 (≈1.2 g/l dry weight of cells). The fermentation was carried out under oxygen limiting conditions with 50 ml of medium in a 125 ml flask, agitation at 100 RPM, and at 30° C. For this fermentation, a starting concentration of 40 g 1⁻¹ glucose and 100 g 1⁻¹ xylose was used.

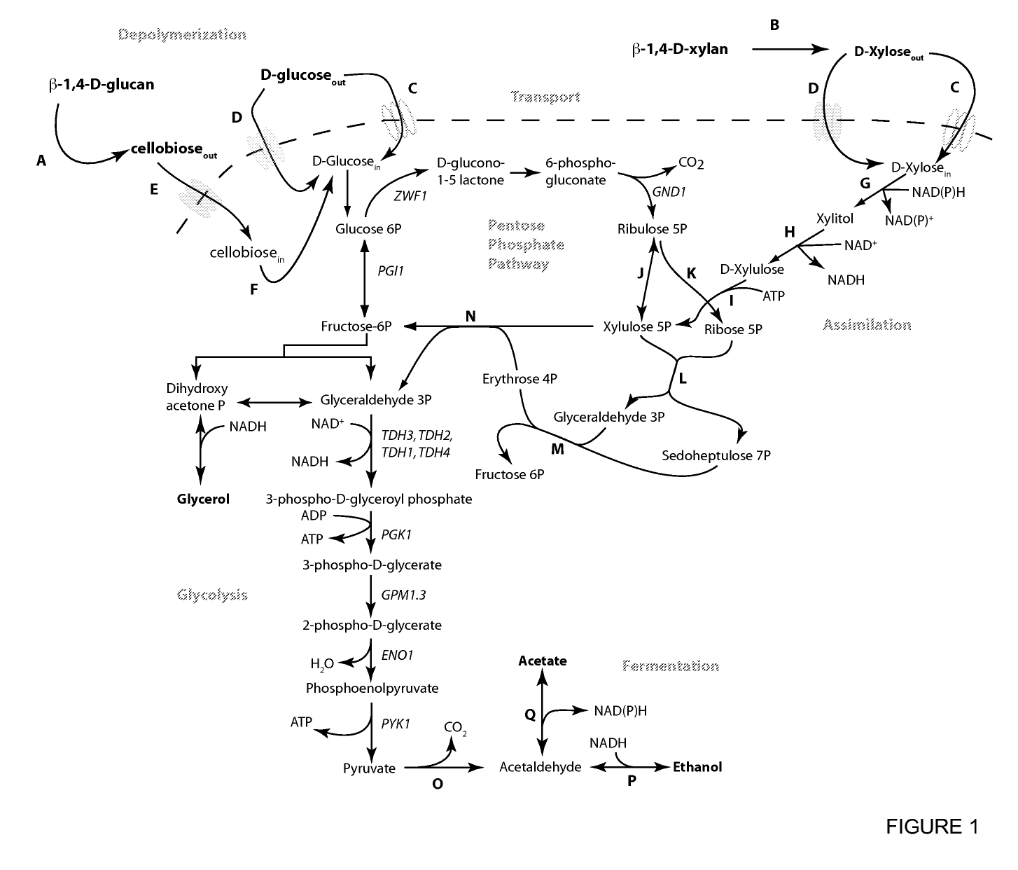

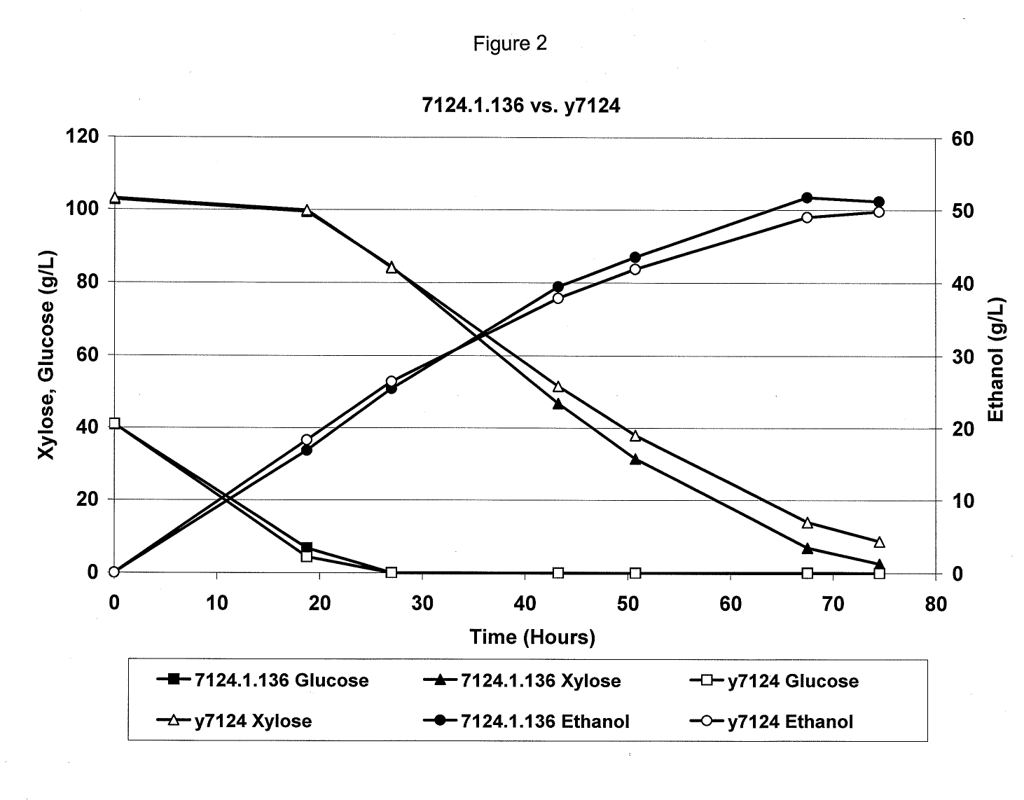

In shake flask trials, 7124.1.136 was able to utilize xylose at a faster rate than the parental strain, 2.31 g/l·h vs. 1.99 g/l·h, a 16.1% increase. A higher yield of ethanol was obtained by 7124.1.136 (51.73 g/l) than by NRRL Y-7124 (49.01 g/l), a 5.5% increase (FIG. 2).

pSDM29 was constructed to contain a synthetic polynucleotide encoding the P. stipitis SUT4 protein under control of the constitutive P. stipitis TDH3 promoter and the native SUT4 terminator. Approximately 100 μg of plasmid was linearized using the restriction enzymes XmaI and XhoI, ethanol precipitated, resuspended in water, creating a fragment that could be directly inserted into the P. stipitis genome. The digested construct was then transformed using a LiAc protocol (2) into 7124.1.136, creating 7124.1.144, into 7124.1.158 creating 7124.1.182, 7124.1.183, 7124.1.184, 7124.1.185, 7124.1.186, and 7124.1.187, and into NRRL Y-7124 creating 7124.2.345, 7124.2.346, 7124.2.347, 7124.2.348, 7124.2.349, 7124.2.350, 7124.2.351, 7124.2.352, 7124.2.353, and 7124.2.354.

Transformants were selected via growth on YPD plates containing 50 μg/ml nourseothricin and dextrose (2%). Colonies were grown overnight in YPD+50 μg/ml nourseothricin liquid medium, genomic DNA was prepped and evaluated by PCR to confirm integration of the fragment.

The NAT1 gene was removed by transforming the transformants with pJML545 (7). Transformants were selected on YPD plates containing 50 μg/ml zeocin and dextrose (2%). Colonies were patched onto YPD and YPD nourseothricin plates to confirm excision of the NAT1 marker.

Shake flask fermentation of 7124.1.144. Cultures were started by inoculating a swath of colonies into 50 ml YPX (2% xylose) and grown overnight. The following morning, duplicate flasks were inoculated to a starting OD600 of 7.0 (≈1.0 g/l dry weight of cells). The fermentation was carried out under oxygen limiting conditions with 50 ml of medium in a 125 ml flask, agitation at 100 RPM, and at 30° C. A modified defined minimal medium was used containing trace metal elements and vitamins, which is based on that described by Verduyn et al. (10). For this fermentation, a starting concentration of 40 g 1⁻¹ glucose and 100 g 1⁻¹ xylose was used.

Bioreactor fermentation of 7124.1.144. A 3 L bioreactor scale-up fermentation was performed to compare strains in a larger scale under controlled conditions. Reactions were performed in 3 L New Brunswick Scientific BioFlo 110 bioreactors with a working volume of 2 L. Reaction conditions were set at 25° C., agitation was set at 500 RPM, pH was set at 5.0 and controlled by additions of either 5 N KOH or 5 N H2SO4. Aeration was controlled at a rate of 0.5 vvm, which corresponded to a rate of 1 l min⁻¹. Cells grew under fully aerobic conditions for 7 hours until an OD600 of approximately 22 was reached (≈3.5 g/l dry weight of cells), at which time the input gas was mixed using a gas proportioner to include 90% pure nitrogen and 10% air, for a final oxygen concentration of approximately 2%.

Cultures were started by inoculating a swath of colonies into 50 ml YPX (4% Xylose) and grown overnight, then recultured in 500 ml YPX (4% Xylose) and grown for an additional 48 hours. Bioreactors were inoculated to a starting OD600 of 9.0 (≈1.4 g/l dry weight of cells), in a defined minimal medium containing trace metal elements and vitamins, which is based on that described by Verduyn et al. (3, 10). For this fermentation, a starting concentration of 40 g 1⁻¹ glucose and 100 g 1⁻¹ xylose was used.

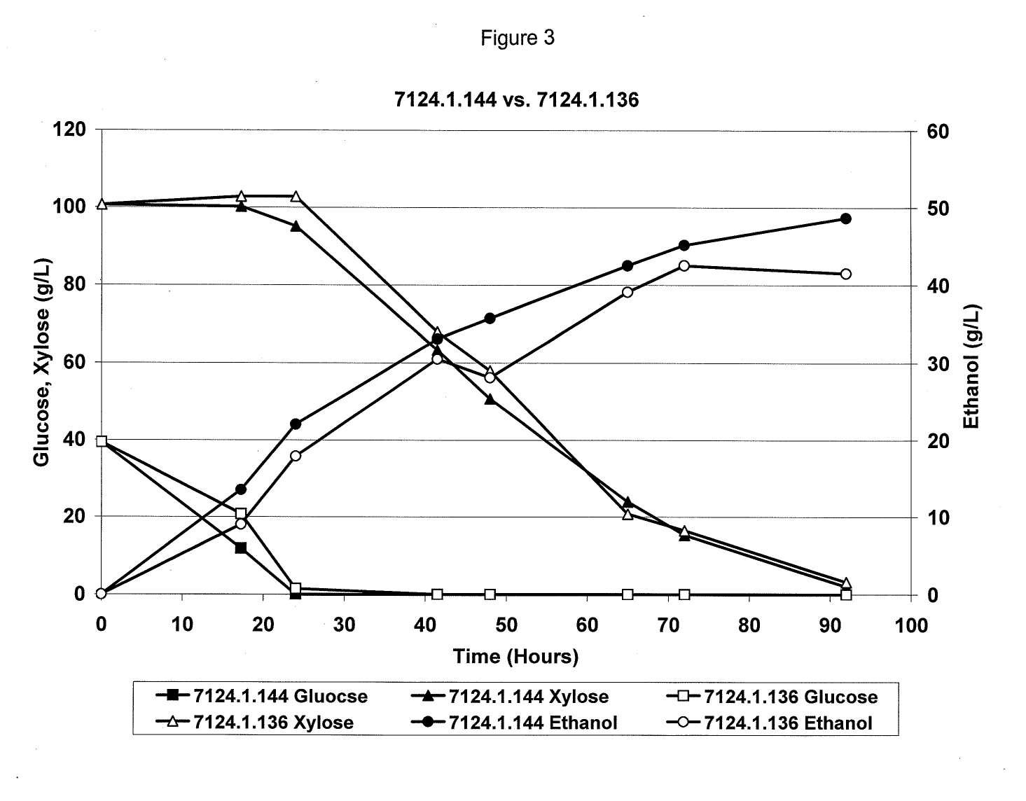

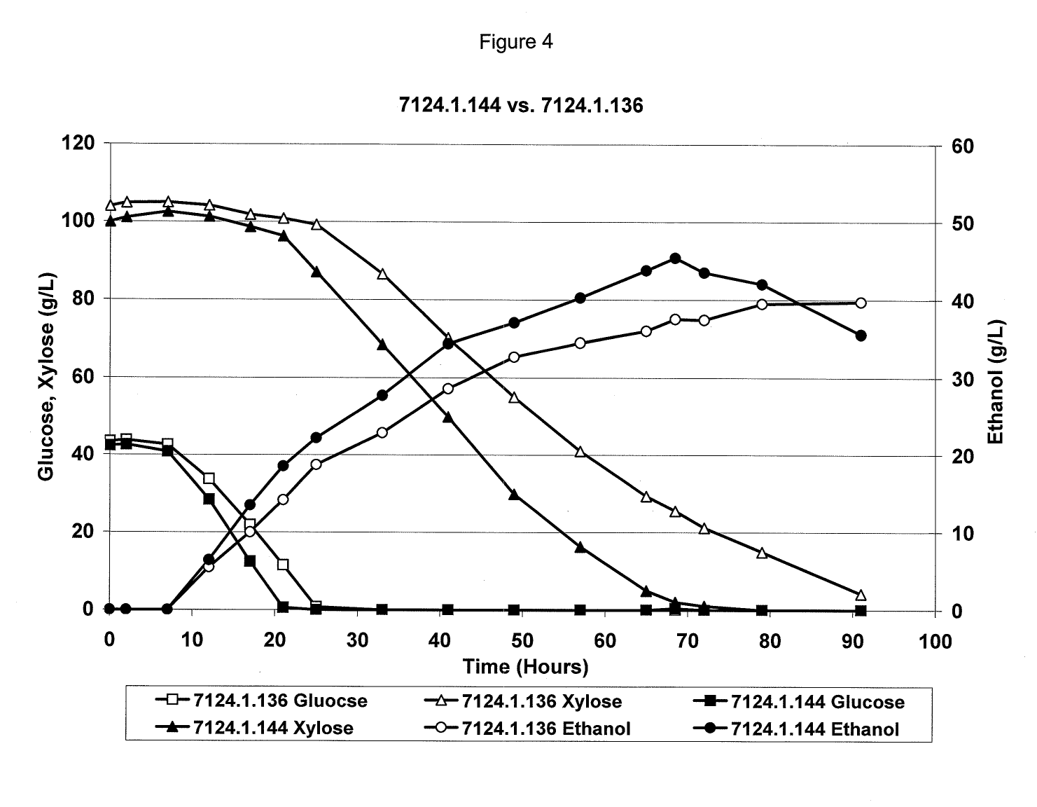

In shake flask trials, 7124.1.144 was able to utilize glucose at a faster rate than the parental strain, 1.60 g/l·h vs. 1.08 g/l·h, which represented an increase of 56%. As a result of the faster glucose use, 7124.1.144 started to use xylose before the parental strain. A higher yield of ethanol was obtained by 7124.1.144 (48.66 g/l) than by 7124.1.136 (41.52 g/l) a 17.2% increase. The specific ethanol yield increased 16.4% in the transformant vs. the parental strain, 0.354 g ethanol/g sugar vs. 0.304 g/g (FIG. 3). Similar results were seen in the bioreactor scale-up, 7124.1.144 utilized both the glucose (2.82 g/l·h vs. 2.22 g/l·h, a 27% increase) and xylose (2.21 g/l·h vs. 1.82 g/l·h, a 21.4% increase) at faster rates than the parental strain. Ethanol production was also higher, resulting in a yield of 45.34 g/l for 7124.1.144 while 7124.1.136 had a yield of 39.49 g/l ethanol, a 14.8% increase (FIG. 4).

pSDM32 was constructed to contain the P. stipitis genes: XYL1 fused to the P. stipitis FAS2 promoter and terminator; XYL2 fused to the P. stipitis TDH3 promoter and terminator; and a synthetic polynucleotide encoding the P. stipitis SUT4 protein under control of the P. stipitis TDH3 promoter and the native SUT4 terminator. Approximately 100 μg of plasmid was linearized using the restriction enzyme NotI, ethanol precipitated, resuspended in water, creating a fragment that could be directly inserted into the P. stipitis genome. The digested construct was then transformed using a LiAc protocol (2) into NRRL Y-7124, creating 7124.2.344.

Transformants were selected via growth on YPD plates containing 50 μg/ml nourseothricin and dextrose (2%). Colonies were grown overnight in YPD+50 μg/ml nourseothricin liquid medium, genomic DNA was prepped and evaluated by PCR to confirm integration of the fragment.

The NAT1 gene was removed by transforming the transformants with approximately 10 μg of pJML545 (7). Transformants were selected on YPD plates containing 50 μg/ml zeocin and dextrose (2%). Colonies were patched onto YPD and YPD nourseothricin plates to confirm excision of the NATI marker.

Shake flask fermentation of 7124.2.344. Cultures were started by inoculating a swath of colonies into 50 ml YPX (2% xylose) and grown overnight. The following morning, triplicate flasks were inoculated to a starting OD600 of 7.0 (≈1.0 g/l dry weight of cells). The fermentation was carried out under oxygen limiting conditions with 50 ml of medium in a 125 ml flask, agitation at 100 RPM, and at 30° C. A modified defined minimal medium was used containing trace metal elements and vitamins, which is based on that described by Verduyn et al. (10). For this fermentation, a starting concentration of 40 g 1⁻¹ glucose and 100 g 1⁻¹ xylose was used.

Bioreactor fermentation of 7124.2.344. A 3 L bioreactor scale-up fermentation was performed to compare strains in a larger scale under controlled conditions. Reactions were performed in 3 L New Brunswick Scientific BioFlo 110 bioreactors with a working volume of 2 L. Reaction conditions were set at 25° C., agitation was set at 500 RPM, pH was set at 5.0 and controlled by additions of either 5 N KOH or 5 N H2SO4. Aeration was controlled at a rate of 0.5 vvm, which corresponded to a rate of 1 l min⁻¹. Cells grew under fully aerobic conditions for 4.5 hours until an OD600 of approximately 18 was reached (≈2.9 g/l dry weight of cells), at which time the input gas was mixed using a gas proportioner to include 90% pure nitrogen and 10% air, for a final oxygen concentration of approximately 2%.

Cultures were started by inoculating a swath of colonies into 50 ml YPX (4% xylose) and grown overnight, then recultured in 500 ml YPX (4% xylose) and grown for an additional 48 hours. Bioreactors were inoculated to a starting OD600 of 8.5 (≈1.3 g/l dry weight of cells), in a defined minimal medium containing trace metal elements and vitamins, which is based on that described by Verduyn et al. (10) (3). For this fermentation, a starting concentration of 40 g 1⁻¹ glucose and 100 g 1⁻¹ xylose was used.

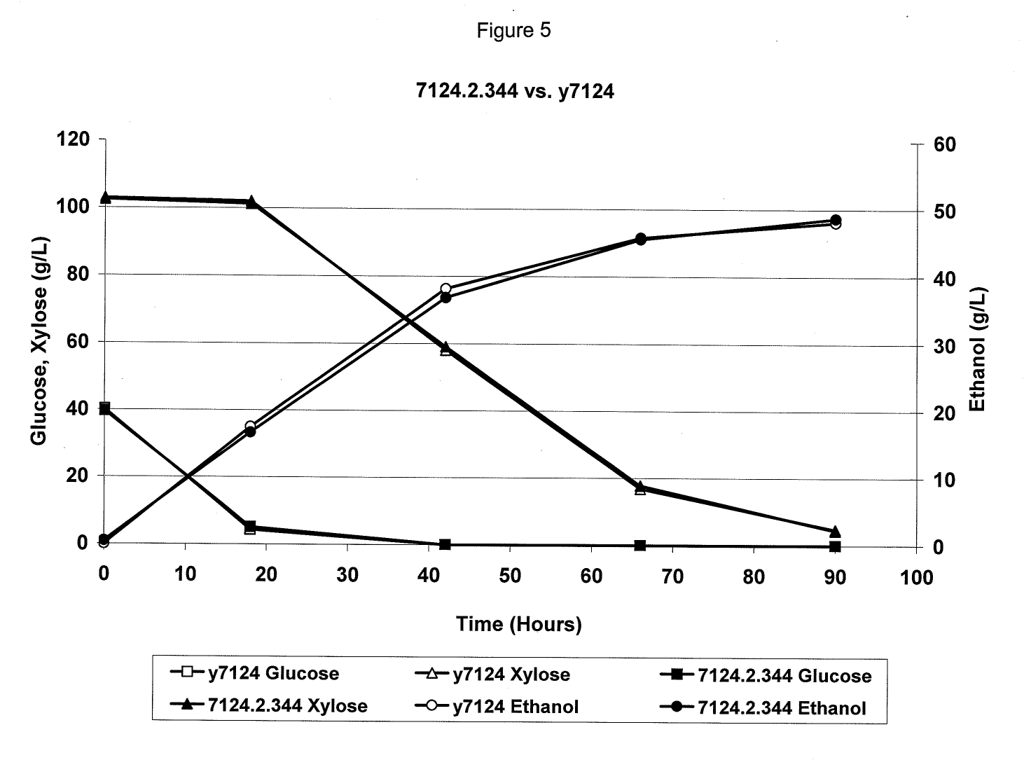

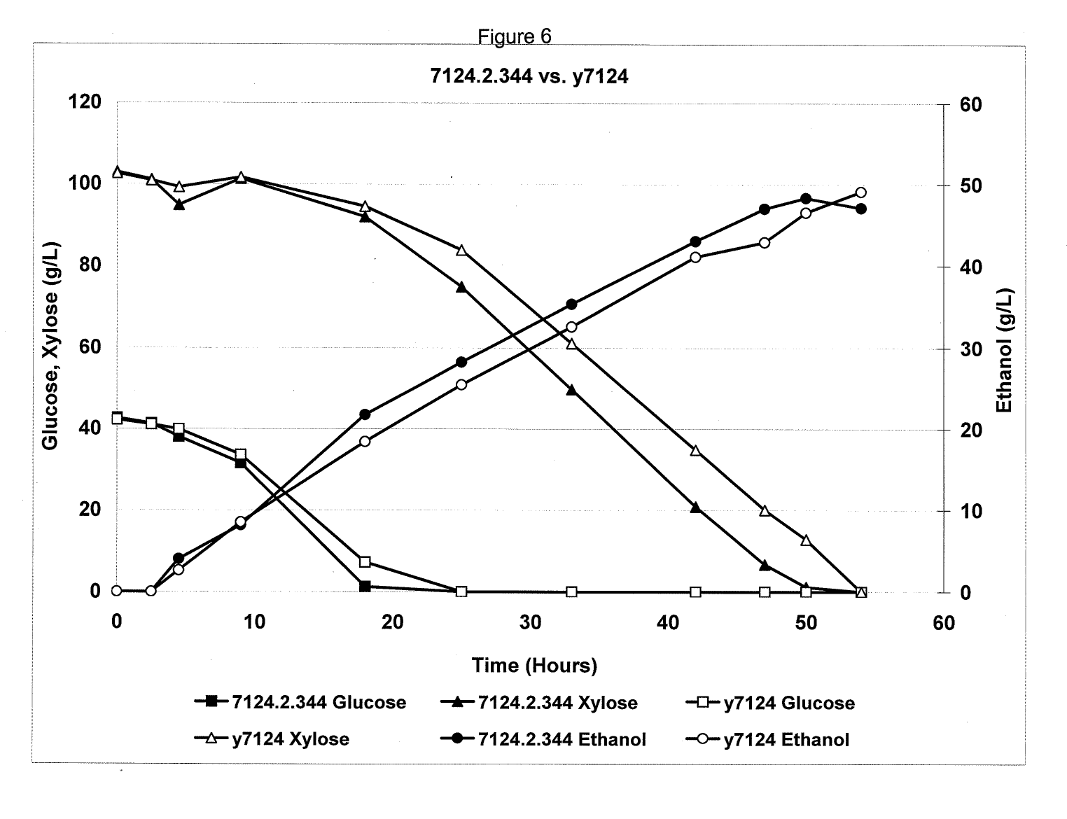

In shake flask trials, 7124.2.344 was able to utilize xylose at a faster rate (1.14 g/l·h vs. 1.11 g/l·h, a 2.7% increase) than the parental strain. A higher yield of ethanol was obtained by 7124.2.344 (48.6 g/l) than by NRRL Y-7124 (48.0 g/l), a 1.3% increase (FIG. 5). The 3 l bioreactor scale-up resulted in 7124.2.344 using both glucose (2.58 g/l·h vs. 2.18 g/l·h, an 18.3% increase) and xylose (2.94 g/l·h vs. 2.57 g/l·h, a 14.4% increase) at faster rates than the parental strain NRRL Y-7124. The ethanol yield after 50 hours was also higher for 7124.2.344, 48.32 g/l versus 46.54 g/l for NRRL Y-7124, a 3.8% increase (FIG. 6).

pSDM24 was constructed to contain the P. stipitis genes: XYL1 fused to the P. stipitis FAS2 promoter and terminator; XYL2 fused to the P. stipitis TDH3 promoter and terminator; and HXT4 gene fused the P. stipitis TDH3 promoter. Approximately 100 μg of plasmid was linearized using the restriction enzyme SacII, ethanol precipitated, resuspended in water, creating a fragment that could be directly inserted into the P. stipitis genome. The digested construct was then transformed using a LiAc protocol into NRRL Y-7124, creating 7124.2.474.

Transformants were selected via growth on YPD plates containing 50 μg/ml nourseothricin and dextrose (2%). Colonies were grown overnight in YPD+50 μg/ml nourseothricin liquid medium, genomic DNA was prepped and evaluated by PCR to confirm integration of the fragment.

The NAT1 gene was removed by transforming the transformants with approximately 10 μg of pJML545 (7). Transformants were selected on YPD plates containing 50 μg/ml zeocin and dextrose (2%). Colonies were patched onto YPD and YPD nourseothricin plates to confirm excision of the NAT1 marker.

Fermentation of 7124.2.474. Cultures were started by inoculating a swath of colonies into 50 ml YPX (2% xylose) and grown overnight. The following morning, triplicate flasks were inoculated to a starting OD600 of 7.5 (≈1.2 g/l dry weight of cells). The fermentation was carried out under oxygen limiting conditions with 50 ml of medium in a 125 ml flask, agitation at 100 RPM, and at 30° C. A modified defined minimal medium was used containing trace metal elements and vitamins, which is based on that described by Verduyn et al. (10). For this fermentation, a starting concentration of 40 g 1⁻¹ glucose and 100 g 1⁻¹ xylose was used.

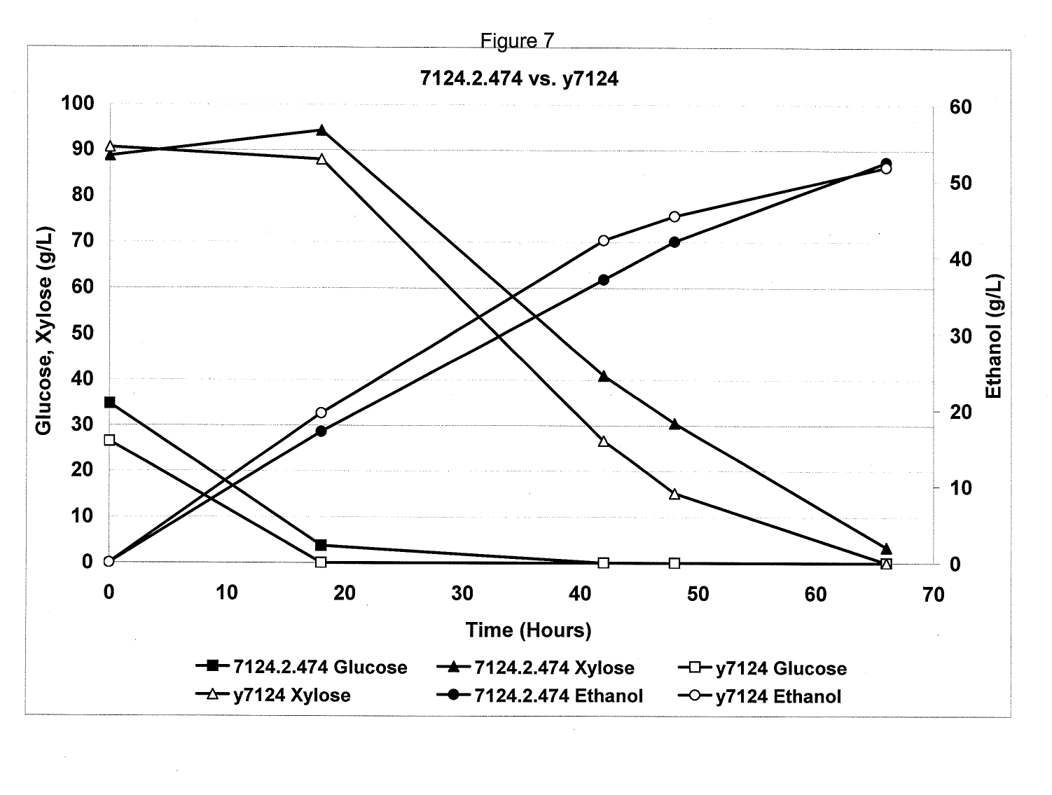

7124.2.474 was able ferment glucose and xylose to ethanol with a specific yield of 0.383 g ethanol produced/g sugar used, compared to a yield of 0.37 g/g for the parental strain, a 3.5% increase. 7124.2.474 failed to produce any xylitol during the 66 hour fermentation, while the control strain did produce xylitol during the fermentation (FIG. 7).

pSDM20 was constructed to contain the P. stipitis genes: XYL1 fused to the P. stipitis FAS2 promoter and terminator; XYL2 fused to the P. stipitis TDH3 promoter and terminator; and XYL3 fused to the P. stipitis ZWF1 promoter and terminator. Approximately 100 μg of plasmid was linearized using the restriction enzymes SacII and PvuII, ethanol precipitated, resuspended in water, creating a fragment that could be directly inserted into the P. stipitis genome. The digested construct was then transformed into 7124.1.136 using a LiAc protocol, creating 7124.1.158, 7124.1.159, 7124.1.160, 7124.1.161, 7124.1.162, and 7124.1.163, containing P. stipitis XYL123, and into a pool of Y-7124 pSDM29 transformants, creating 7124.2.415 and 7124.2.418.

Transformants were selected via growth on YPD plates containing 50 μg/ml nourseothricin and dextrose (2%). Colonies were grown overnight in YPD+50 μg/ml nourseothricin liquid medium, genomic DNA was prepped and evaluated by PCR to confirm integration of the fragment.

The NAT1 gene was removed by transforming the transformants with approximately 10 μg of pJML545 (7). Transformants were selected on YPD plates containing 50 μg/ml zeocin and dextrose (2%). Colonies were patched onto YPD and YPD nourseothricin plates to confirm excision of the NATI marker.

Screening 7124.1.158, 7124.1.159, 7124.1.160, 7124.1.161, 7124.1.162, and 7124.1.163 in shake flasks. Cultures were started by inoculating a swath of colonies into 50 ml YPX (2% xylose) and grown overnight. The following morning, duplicate flasks were inoculated to a starting OD600 of 7.5 (≈1.2 g/l dry weight of cells). The fermentation was carried out under oxygen limiting conditions with 50 ml of medium in a 125 ml flask, agitation at 100 RPM, and at 30° C. A modified defined minimal medium was used containing trace metal elements and vitamins, which is based on that described by Verduyn et al. (10). For this fermentation, a starting concentration of 40 g 1⁻¹ glucose and 100 g 1⁻¹ xylose was used.

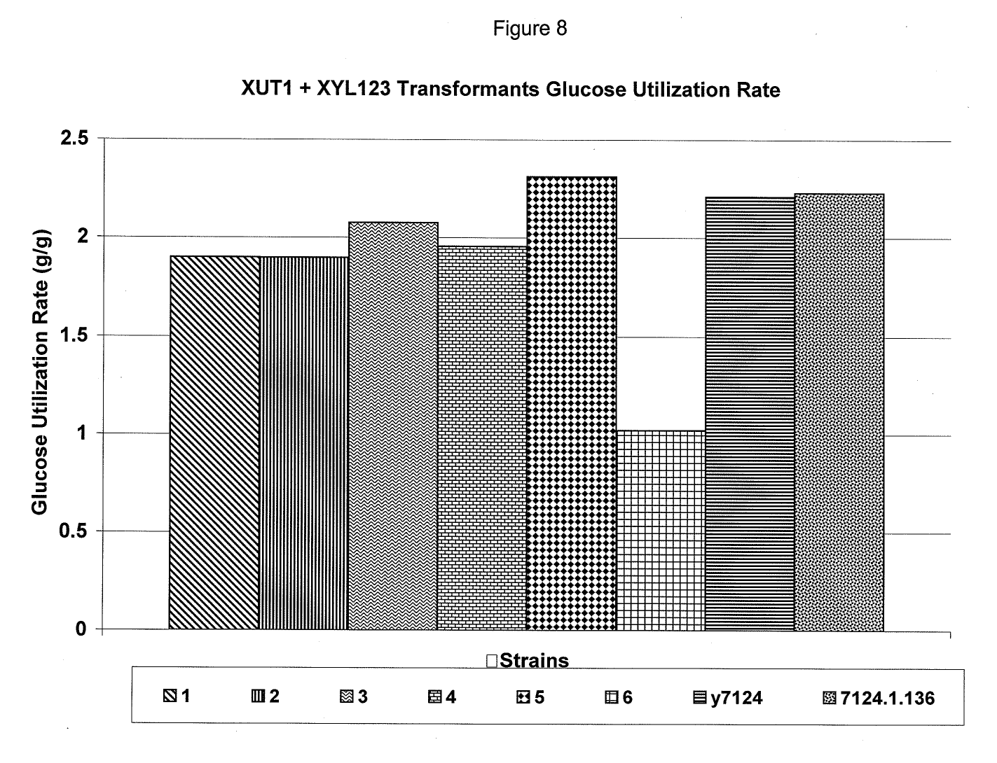

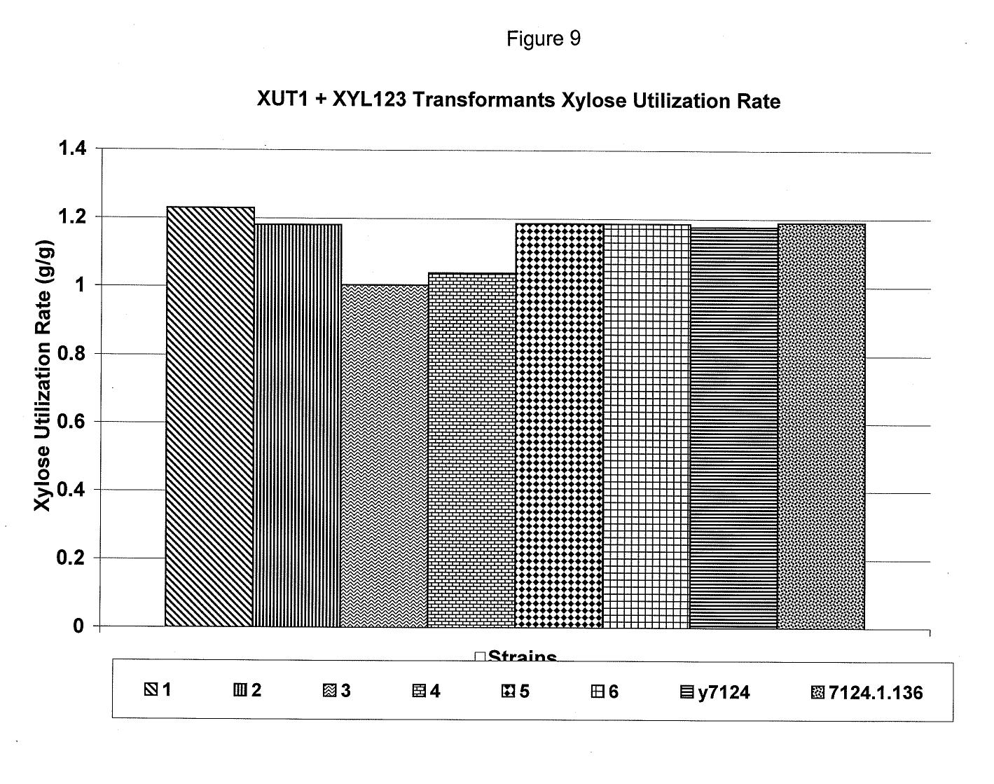

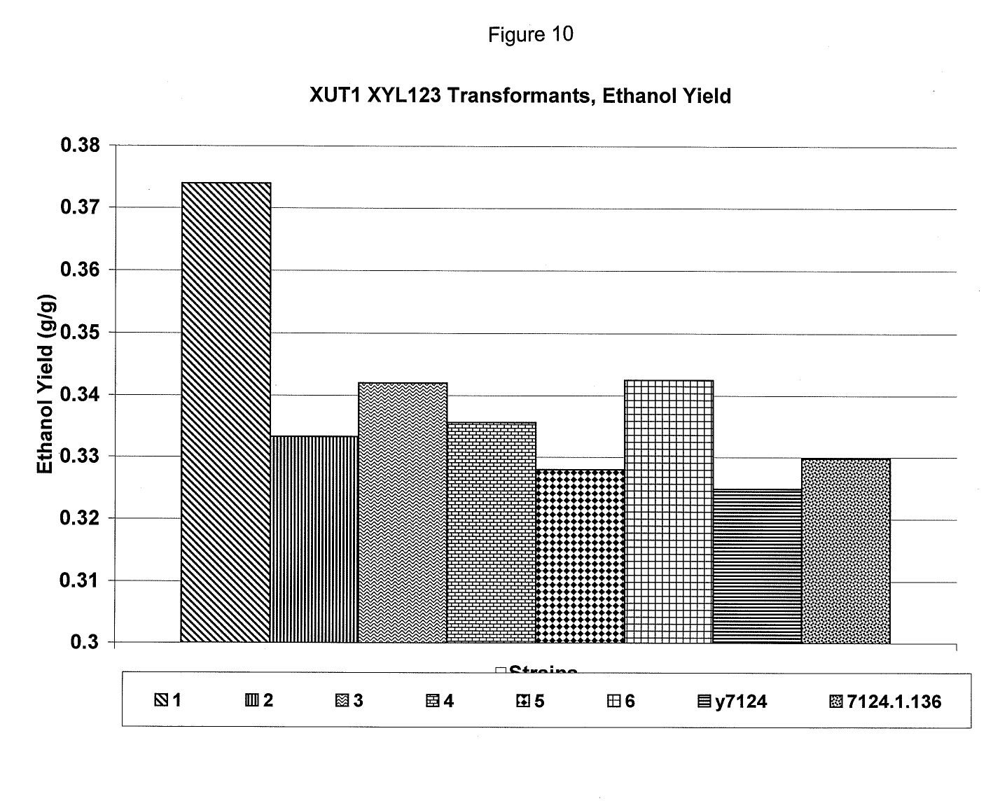

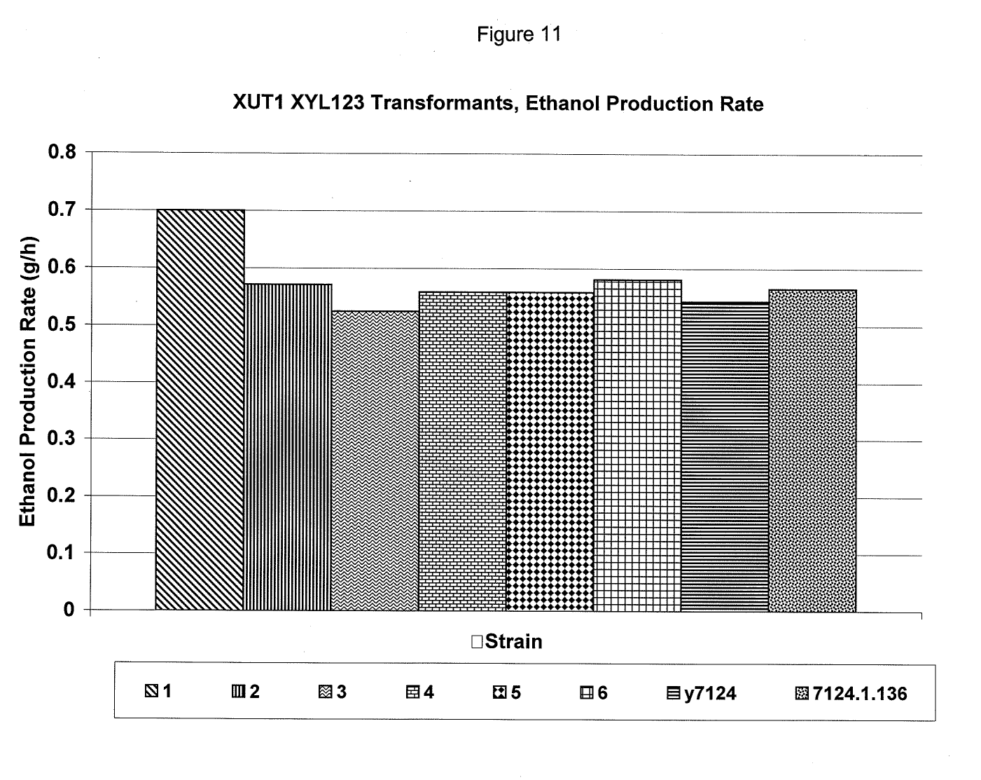

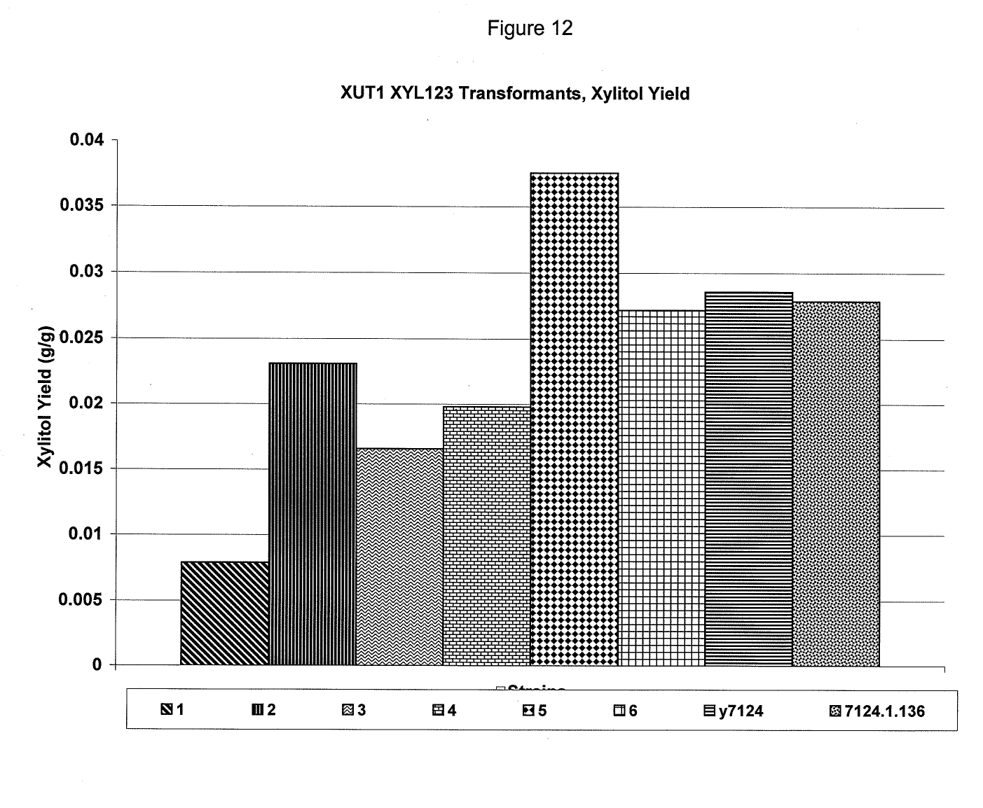

Results of shake flask screen of 7124.1.158, 7124.1.159, 7124.1.160, 7124.1.161, 7124.1.162, and 7124.1.163. The glucose utilization rate ranged from 1.021 g/l·h to 2.312 g/l·h, both rates were those of different transformants (FIG. 8). The xylose utilization rate ranged from 1.005 g/l·h to 1.229 g/l·h, both rates were those of different transformants (FIG. 9). The specific ethanol yield ranged from 0.325 g/g to 0.374 g/g, the lower figure was from the NRRL Y-7124, the higher from a transformant (FIG. 10). The ethanol production rate values ranged from 0.525 g/h to 0.700 g/h, both of these figures were from transformants (FIG. 11). The xylitol production rate values ranged from 0.008 g/g to 0.038 g/g, both of these values were from transformants (FIG. 12). Strain 7124.1.158 had the highest xylose utilization rate, the highest specific ethanol yield, the highest ethanol production rate, and the lowest xylitol production rate. Strain, 7124.1.158, was further evaluated.

Bioreactor fermentation of 7124.1.158. A 3 L bioreactor scale-up fermentation was performed to compare strains in a larger scale under controlled conditions. Reactions were performed in 3 L New Brunswick Scientific BioFlo 110 bioreactors with a working volume of 2 L. Reaction conditions were set at 25° C., pH was set at 5.0 and controlled by additions of either 5 N KOH or 5 N H2SO4. Aeration was controlled at a rate of 0.5 vvm, which corresponded to a rate of 1 l min⁻¹. Cells grew with 10% dissolved oxygen and a variable agitation rate (50-300 RPM) for 8 hours until an OD600 of approximately 18 was reached (≈2.9 g/l dry weight of cells), at which time the input gas was mixed using a gas proportioner to include 50% pure nitrogen and 50% air, for a final oxygen concentration of approximately 10%, and the agitation rate was increased to 500 RPM.

Cultures were started by inoculating a swath of colonies into 50 ml YPX (4% xylose) and grown overnight, then recultured in 500 ml YPX (4% xylose) and grown for an additional 48 hours. Bioreactors were inoculated with unwashed cells to a starting OD600 of 8.0 (≈1.3 g/l dry weight of cells), in a defined minimal medium containing trace metal elements and vitamins, which is based on that described by Verduyn et al. (10). For this fermentation, a starting concentration of 40 g 1⁻¹ glucose and 100 g 1⁻¹ xylose was used.

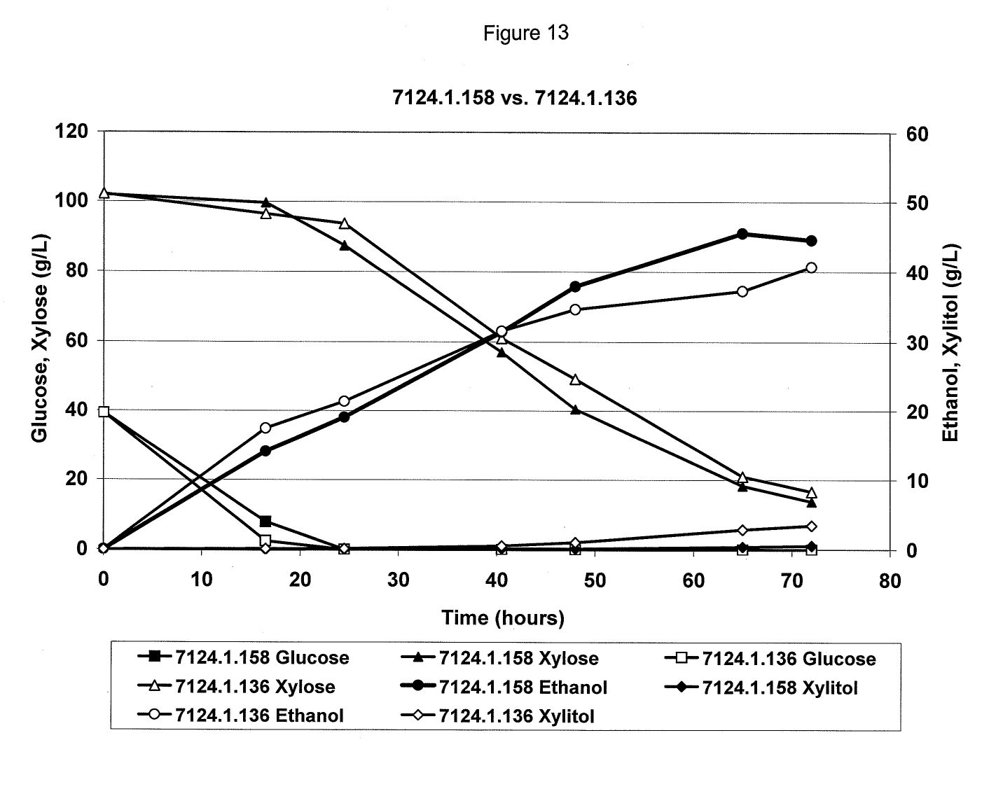

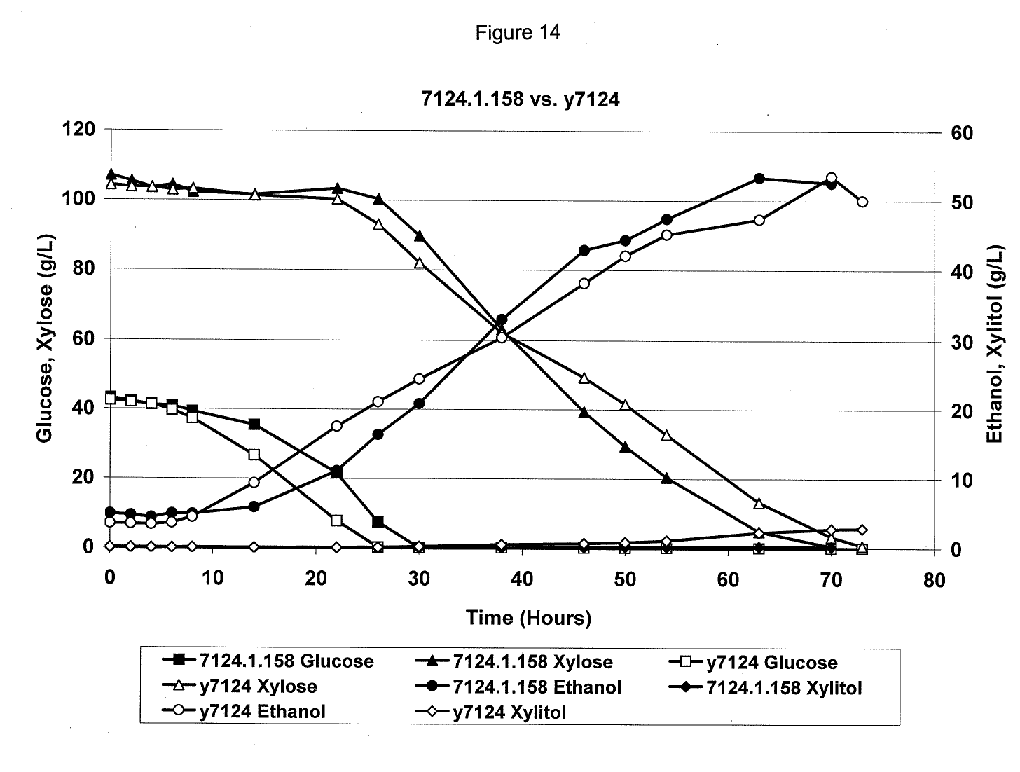

In shake flask trials, 7124.1.158 was able to utilize xylose (2.58 g/l·h vs. 2.16 g/l·h, a 22% increase) at a faster rate than the parental strain. A higher yield of ethanol was obtained after 65 hours of fermentation by 7124.1.158 (45.5 g/l) than by 7124.136 (37.28 g/l), a 22% increase. An increase of 19.5% in the specific ethanol yield was seen in 7124.1.158 (0.374 g ethanol/g sugar) vs. the parental strain (0.313 g ethanol/g sugar) (FIG. 13). The bioreactor scale-up resulted in 7124.1.158 utilizing xylose (1.88 g/l·h vs. 1.50 g/l·h, 19.4% increase) at a faster rate than the control NRRL Y-7124 strain. 7124.1.158 had a higher ethanol yield than the NRRL Y-7124 control strain at 63 hours; 53.31 versus 47.28 g/l ethanol, a 12.8% increase. An increase of 5.6% in the specific ethanol yield was seen in 7124.1.158 (0.394 g ethanol/g sugar) vs. the control strain (0.373 g ethanol/g sugar). The xylitol yield was lower in 7124.1.158 (0.22 g/l) than in NRRL Y-7124 (2.40 g/l), a 91% decrease (FIG. 14).

Analysis of 7124.1.158 in 3 L bioreactors, grown under different oxygen limitation conditions. Reactions were performed in 3 L New Brunswick Scientific BioFlo 110 bioreactors with a working volume of 2 L. Reaction conditions were set at 25° C., pH was set at 5.0 and controlled by additions of either 5 N KOH or 5 N H2SO4. Aeration was controlled at a rate of 0.5 vvm, which corresponded to a rate of 1 l min⁻¹. Condition 1: Cells grew with 10% dissolved oxygen and a variable agitation rate (50-300 RPM) for 6 hours until an OD600 of approximately 18 was reached (≈2.9 g/l dry weight of cells), at which time the input gas was mixed using a gas proportioner to include 50% pure nitrogen and 50% air, for a final oxygen concentration of approximately 10%, and the agitation rate was increased to 500 RPM. Condition 2: Cells grew under fully aerobic conditions, with an agitation rate of 500 RPM, for 6 hours until an OD600 of approximately 18 was reached (≈2.9 g/l dry weight of cells), at which time the input gas was mixed using a gas proportioner to include 50% pure nitrogen and 50% air, for a final oxygen concentration of approximately 10%.

Cultures were started by inoculating a swath of colonies into 3 ml YPX (4% xylose) and grown overnight, then recultured in 350 ml YPX (4% xylose), grown for an additional 72 hours, and then diluted with an additional 350 ml YPX (4% xylose), and grown overnight. Bioreactors were inoculated with unwashed cells to a starting OD600 of 7.7 (≈1.2 g/l dry weight of cells), in a defined minimal medium containing trace metal elements and vitamins, which is based on that described by Verduyn et al. (10). For this fermentation, a starting concentration of 40 g 1⁻¹ glucose and 100 g 1⁻¹ xylose was used.

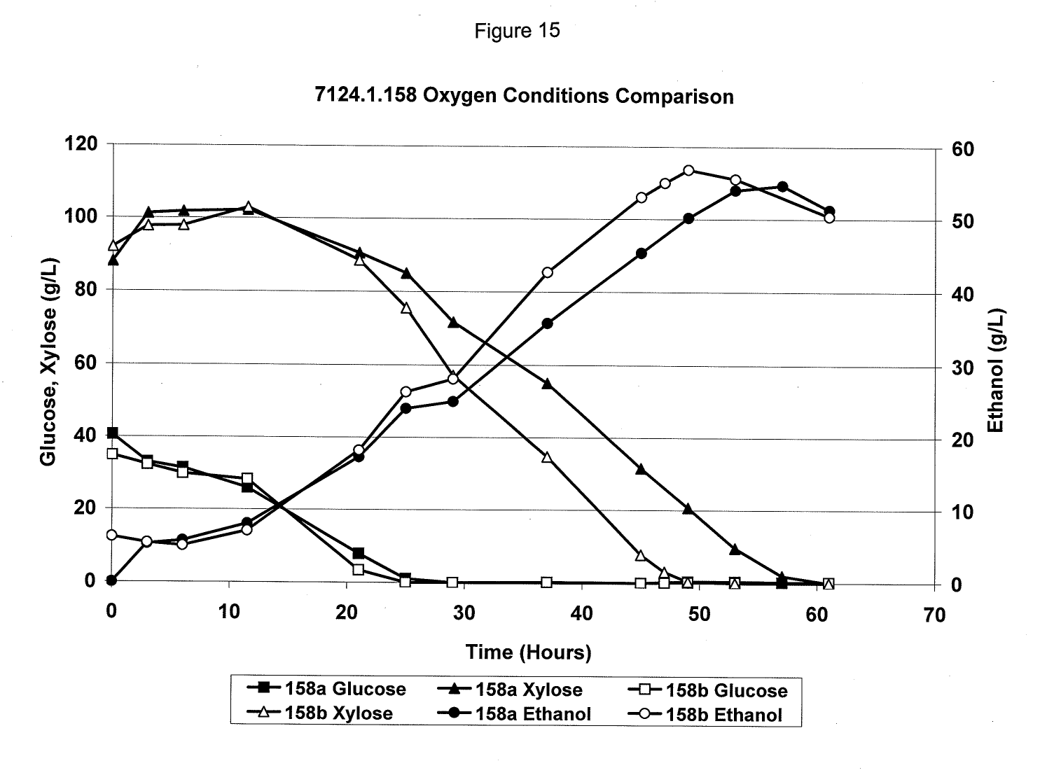

Results of oxygen comparison: Cells grown under oxygen condition 2, had a faster xylose utilization rate than condition 1 grown cells 3.368 g/l·h vs. 2.532 g/l·h, a 33.0% increase. Condition 2 produced an ethanol yield of 56.81 g/l vs. 54.62 g/l, a 4.0% increase, with an ethanol production rate increase of 20.9% (1.159 g/l·h vs. 0.958 g/l·h). The specific ethanol production rate increased 2.5% for cells grown in condition 2, 0.406 g/g vs. 0.396 g/g (FIG. 15).

Fermentation of 7124.2.415. Cultures were started by inoculating a swath of colonies into 100 ml of modified defined minimal medium containing trace metal elements and vitamins, which is based on that described by Verduyn et al. (10), with sugar a concentration of 40 g 1⁻¹ glucose and 100 g 1⁻¹ xylose. After 96 hours, triplicate flasks were inoculated to a starting OD600 of 8.0 (≈1.3 g/l dry weight of cells). The fermentation was carried out under oxygen limiting conditions with 50 ml of medium, as above, in a 125 ml flask, agitation at 100 RPM, and at 30° C.

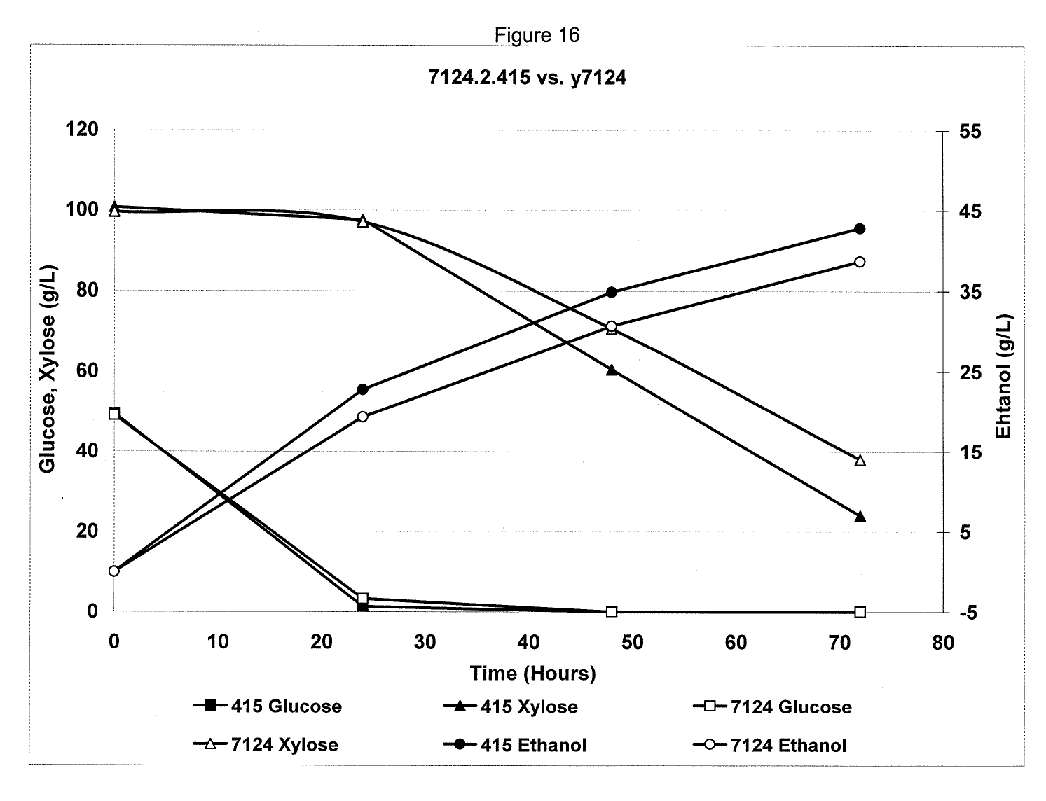

In shake flask trials, 7124.2.415 was able to utilize both glucose (2.00 g/l·h vs. 1.90 g/l·h, a 5.2% increase) and xylose (1.53 g/l·h vs. 1.22 g/l·h, a 25.4% increase) at faster rates than NRRL Y-7124. A higher yield of ethanol was obtained after 72 hours of fermentation by 7124.2.415 (42.7 g/l) than by NRRL Y-7124 (38.7 g/l), a 10.3% increase (FIG. 16).

Analysis of 7124.2.418 in 3 L bioreactors, grown under different oxygen limitation conditions. Reactions were performed in 3 L New Brunswick Scientific BioFlo 110 bioreactors with a working volume of 2 L. Reaction conditions were set at 25° C., pH was set at 5.0 and controlled by additions of either 5 N KOH or 5 N H2SO4. Aeration was controlled at a rate of 0.5 vvm, which corresponded to a rate of 1 l min⁻¹. Condition 1: Cells grew with 10% dissolved oxygen and a variable agitation rate (50-300 RPM) for 6 hours until an OD600 of approximately 18 was reached (≈2.9 g/l dry weight of cells), at which time the input gas was mixed using a gas proportioner to include 50% pure nitrogen and 50% air, for a final oxygen concentration of approximately 10%, and the agitation rate was increased to 500 RPM. Condition 2: Cells grew under fully aerobic conditions, with an agitation rate of 500 RPM, for 6 hours until an OD600 of approximately 18 was reached (≈2.9 g/l dry weight of cells), at which time the input gas was mixed using a gas proportioner to include 50% pure nitrogen and 50% air, for a final oxygen concentration of approximately 10%.

Cultures were started by inoculating a swath of colonies into 3 ml YPX (4% xylose) and grown overnight, then recultured in 350 ml YPX (4% xylose), grown for an additional 72 hours, and then diluted with an additional 350 ml YPX (4% xylose), and grown overnight. Bioreactors were inoculated with unwashed cells to a starting OD600 of 7.7 (≈1.2 g/l dry weight of cells), in a defined minimal medium containing trace metal elements and vitamins, which is based on that described by Verduyn et al. (10). For this fermentation, a starting concentration of 40 g 1⁻¹ glucose and 100 g 1⁻¹ xylose was used.

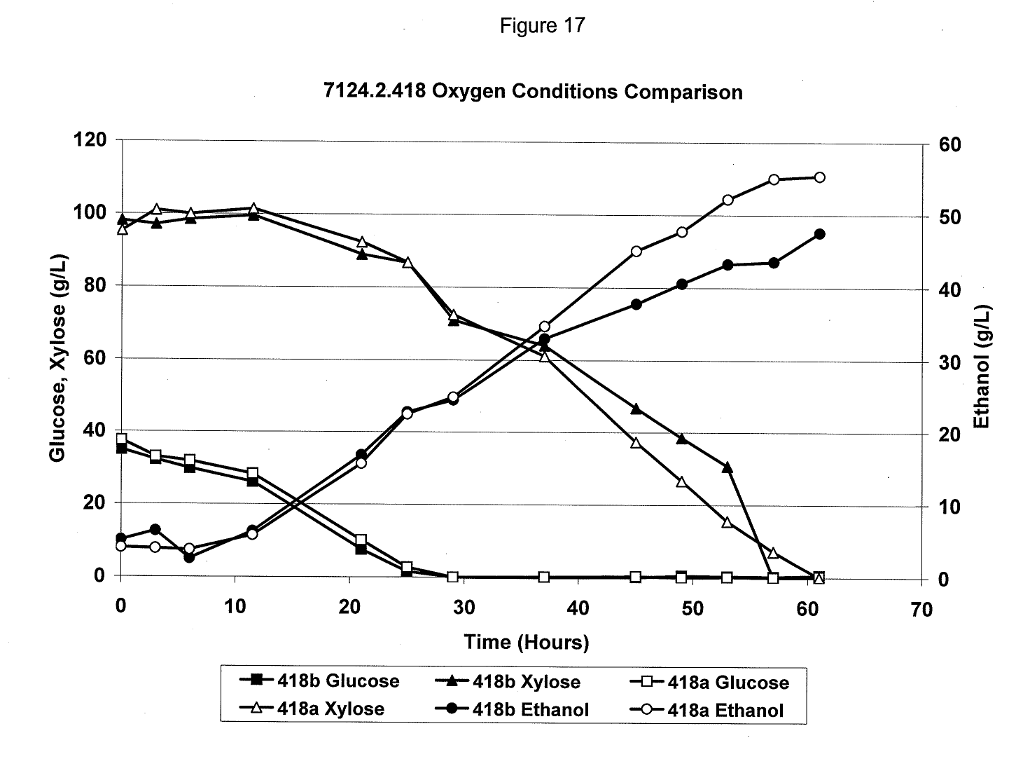

Results of comparison: Cells grown under oxygen condition 1, had an ethanol yield of 55.0 g/l vs. 47.52 g/l, a 15.7% increase, with an ethanol production rate increase of 23.8% (0.965 g/l·h vs. 0.779 g/l·h). The specific ethanol production rate increased 21.8% for cells grown in condition 1, 0.413 g/g vs. 0.339 g/g (FIG. 17).

pSDM21 was constructed to contain a synthetic polynucleotide encoding the Zymomonas mobilis ADH1 protein, fused to the P. stipitis TDH3 promoter and terminator. Approximately 100 μg of plasmid was linearized using the restriction enzymes NotI, ethanol precipitated, resuspended in water, creating a fragment that could be directly inserted into the P. stipitis genome. The digested construct was then transformed using a LiAc protocol (2) into 7124.2.344, creating 7124.2.405, 7124.2.406, 7124.2.407, 7124.2.408, and 7124.2.409 and into 7124.1.144 creating 7124.1.164, 7124.1.165, 7124.1.166, 7124.1.167, 7124.1.168, and 7124.1.169.

Transformants were selected via growth on YPD plates containing 50 μg/ml nourseothricin and dextrose (2%). Colonies were grown overnight in YPD+50 μg/ml nourseothricin liquid medium, genomic DNA was prepped and evaluated by PCR to confirm integration of the fragment.

The NAT1 gene was removed by transforming the transformants with approximately 10 μg of pJML545 (7). Transformants were selected on YPD plates containing 50 μg/ml zeocin and dextrose (2%). Colonies were patched onto YPD and YPD nourseothricin plates to confirm excision of the NATI marker.

Bioreactor fermentation of 7124.2.407. A 3 L bioreactor scale-up fermentation was performed to compare strains in a larger scale under controlled conditions. Reactions were performed in 3 L New Brunswick Scientific BioFlo 110 bioreactors with a working volume of 2 L. Reaction conditions were set at 25° C., agitation was set at 500 RPM, pH was set at 5.0 and controlled by additions of either 5 N KOH or 5 N H2SO4. Aeration was controlled at a rate of 0.5 vvm, which corresponded to a rate of 1 l min⁻¹, cells grew under fully aerobic conditions for 6.5 hours until an OD600 of approximately 22 was reached (≈3.5 g/l dry weight of cells), at which time the input gas was mixed using a gas proportioner to include 90% pure nitrogen and 10% air, for a final oxygen concentration of approximately 2%.

Cultures were started by inoculating a swath of colonies into 50 ml YPX (4% xylose) and grown overnight, then recultured in 500 ml YPX (4% xylose) and grown for an additional 48 hours. Bioreactors were inoculated to a starting OD600 of 5.0 (≈0.8 g/l dry weight of cells), in a defined minimal medium containing trace metal elements and vitamins, which is based on that described by Verduyn et al. (10) For this fermentation, a starting concentration of 40 g 1⁻¹ glucose and 100 g 1⁻¹ xylose was used.

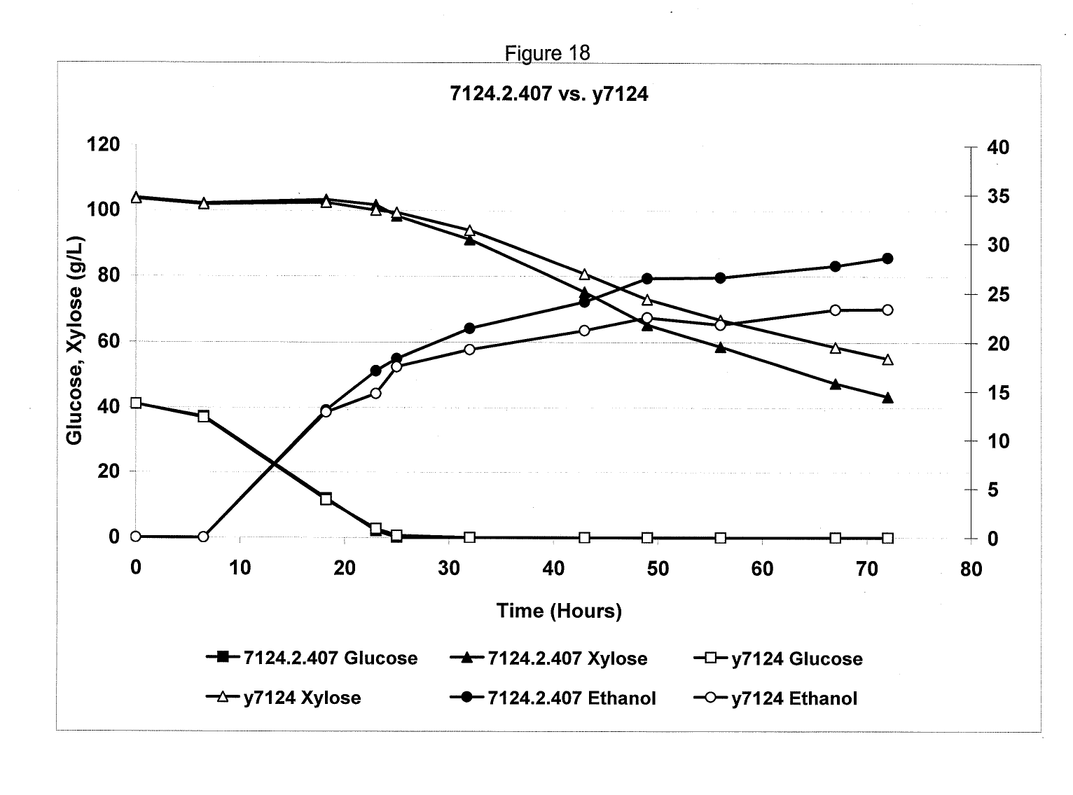

In the bioreactor, 7124.2.407 used xylose (1.19 g/l·h vs. 0.91 g/l·h, a 30.7% increase) faster than NRRL Y-7124, and produced ethanol at a faster rate and reached a higher final concentration than NRRL Y-7124, 28.56 g/l versus 23.37 g/l ethanol, a 22.2% increase. The specific ethanol yield increased 7.3% in 7124.2.407 (0.295 g ethanol/g sugar) vs. NRRL Y-7124 (0.275 g/g) (FIG. 18).

pSDM25 was constructed to contain a synthetic polynucleotide encoding the Zymomonas mobilis ADH1 protein, fused to the P. stipitis TDH3 promoter and terminator, and the HXT4 gene fused the P. stipitis TDH3 promoter. Approximately 100 μg of plasmid was linearized using the restriction enzymes SacII and KpnI, ethanol precipitated, resuspended in water, creating a fragment that could be directly inserted into the P. stipitis genome. The digested construct was then transformed using a LiAc protocol (2) into 7124.1.144, creating 7124.1.155, into a pool of 7124.2.415, 7124.2.416, 7124.2.417, 7124.2.418, and 7124.2.419, creating 7124.2.462, and into NRRL Y-7124 creating 7124.2.469 and 7124.2.470.

Transformants were selected via growth on YPD plates containing 50 μg/ml nourseothricin and dextrose (2%). Colonies were grown overnight in YPD+50 μg/ml nourseothricin liquid medium, genomic DNA was prepped and evaluated by PCR to confirm integration of the fragment.

The NAT1 gene was removed by transforming the transformants with approximately 10 μg of pJML545 (7). Transformants were selected on YPD plates containing 50 μg/ml zeocin and dextrose (2%). Colonies were patched onto YPD and YPD nourseothricin plates to confirm excision of the NATI marker.

Fermentation of 7124.1.155. Cultures were started by inoculating a swath of colonies into 50 ml YPX (2% xylose) and grown overnight. The following morning, duplicate flasks were inoculated to a starting OD600 of 7.5 (≈1.2 g/l dry weight of cells). The fermentation was carried out under oxygen limiting conditions with 50 ml of medium in a 125 ml flask, agitation at 100 RPM, and at 30° C. A modified defined minimal medium was used containing trace metal elements and vitamins, which is based on that described by Verduyn et al. (10) For this fermentation, a starting concentration of 40 g 1⁻¹ glucose and 100 g 1⁻¹ xylose was used.

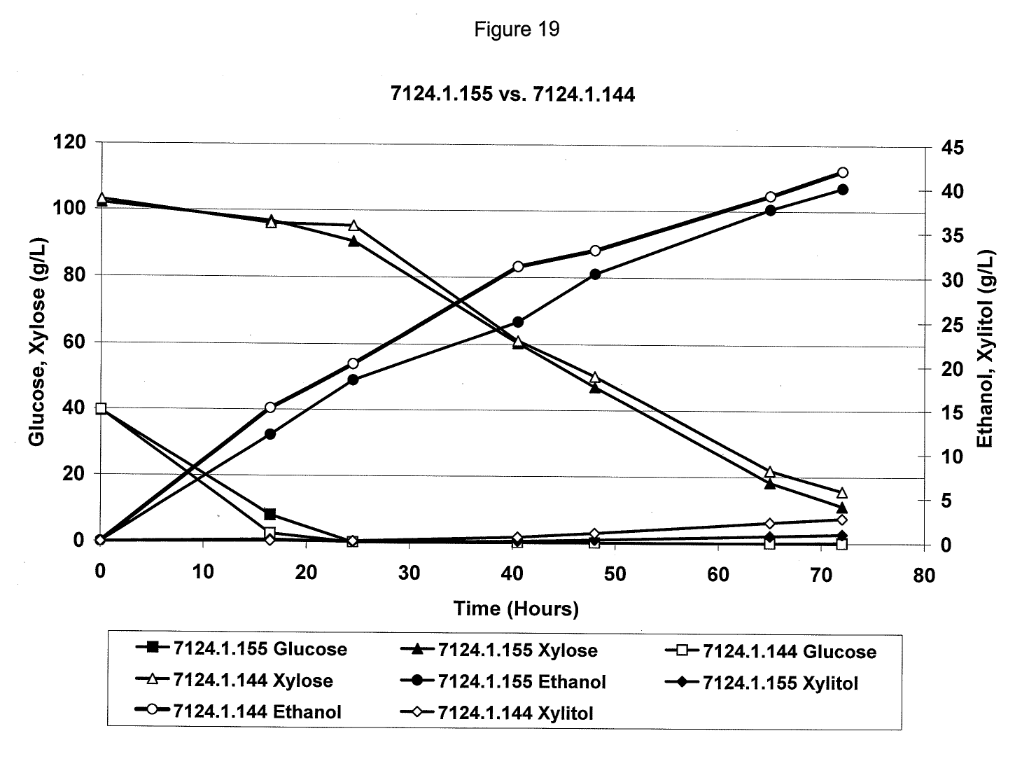

In shake flask trials, 7124.1.155 was able to utilize xylose (1.54 g/l·h vs. 1.45 g/l·h, a 6.2% increase) at faster rate than the parental strain, with decreased xylitol production (1.02 g/l vs. 2.81 g/l, a 63.7% decrease) (FIG. 19).

Fermentation of 7124.2.462. Cultures were started by inoculating a swath of colonies into 100 ml of modified defined minimal medium containing trace metal elements and vitamins, which is based on that described by Verduyn et al. (10), with sugar a concentration of 40 g 1⁻¹ glucose and 100 g 1⁻¹ xylose. After 96 hours, triplicate flasks were inoculated to a starting OD600 of 8.0 (≈1.3 g/l dry weight of cells). The fermentation was carried out under oxygen limiting conditions with 50 ml of medium, as above, in a 125 ml flask, agitation at 100 RPM, and at 30° C.

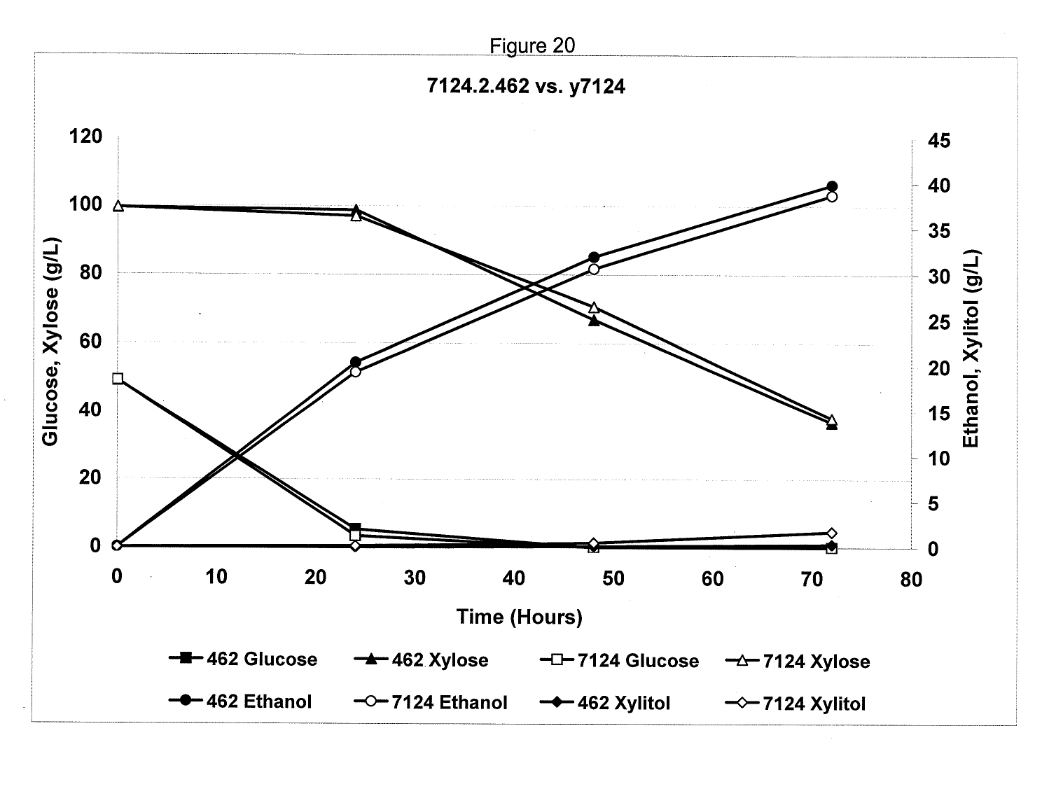

In shake flask trials, 7124.2.462 was able to utilize xylose (1.29 g/l·h vs. 1.22 g/l·h, a 5.7% increase) at a faster rate than NRRL Y-7124. A higher yield of ethanol was obtained after 72 hours of fermentation by 7124.2.462 (39.8 g/l) than by NRRL Y-7124 (38.7 g/l), a 2.8% increase. Xylitol production was decreased by 81.2% in 7124.2.462, which produced 0.32 g/l compared to NRRL Y-7124 which produced 1.71 g/l (FIG. 20).

pSDM31 was constructed to contain the P. stipitis XUT3 gene under control of the constitutive P. stipitis TKT1 promoter and the native XUT3 terminator. Approximately 100 μg of plasmid was linearized using the restriction enzymes NotI and KpnI, ethanol precipitated, resuspended in water, creating a fragment that could be directly inserted into the P. stipitis genome. The digested construct was then transformed using a LiAc protocol (2) into NRRL Y-7124, creating 7124.2.465, 7124.2.466, 7124.2.467, and 7124.2.468, into 7124.1.144 creating 7124.1.176, 7124.1.177, 7124.1.178, 7124.1.179, 7124.1.180, and 7124.1.181, and into a pool of 7124.2.405, 7124.2.406, 7124.2.407, 7124.2.408, and 7124.2.409 creating 7124.2.455, 7124.2.456, 7124.2.457, 7124.2.458, 7124.2.459, and 7124.2.460.

Transformants were selected via growth on YPD plates containing 50 μg/mL nourseothricin and dextrose (2%). Colonies were grown overnight in YPD+50 μg/mL nourseothricin liquid medium, genomic DNA was prepped and evaluated by PCR to confirm integration of the fragment.

The NatI gene was removed by transforming the transformants with approximately 10 μg of pJML545 (7). Transformants were selected on YPD plates containing 50 μg/mL zeocin and dextrose (2%). Colonies were patched onto YPD and YPD nourseothricin plates to confirm excision of the NATI marker.

Shake flask fermentation of 7124.2.465, 7124.2.466, 7124.2.467, and 7124.2.468. Cultures were started by inoculating a swath of colonies into 50 mL YPX (3% xylose) and grown overnight. The following morning, triplicate flasks were inoculated to a starting OD600 of 14.0 (≈1.96 g/l dry weight of cells). The fermentation was carried out under oxygen limiting conditions with 50 mL of medium in a 125 mL flask, agitation at 100 RPM, and at 30° C. A modified defined minimal medium was used containing trace metal elements and vitamins, which is based on that described by Verduyn et al. (10). For this fermentation, a starting concentration of 40 g 1⁻¹ glucose and 100 g 1⁻¹ xylose was used.

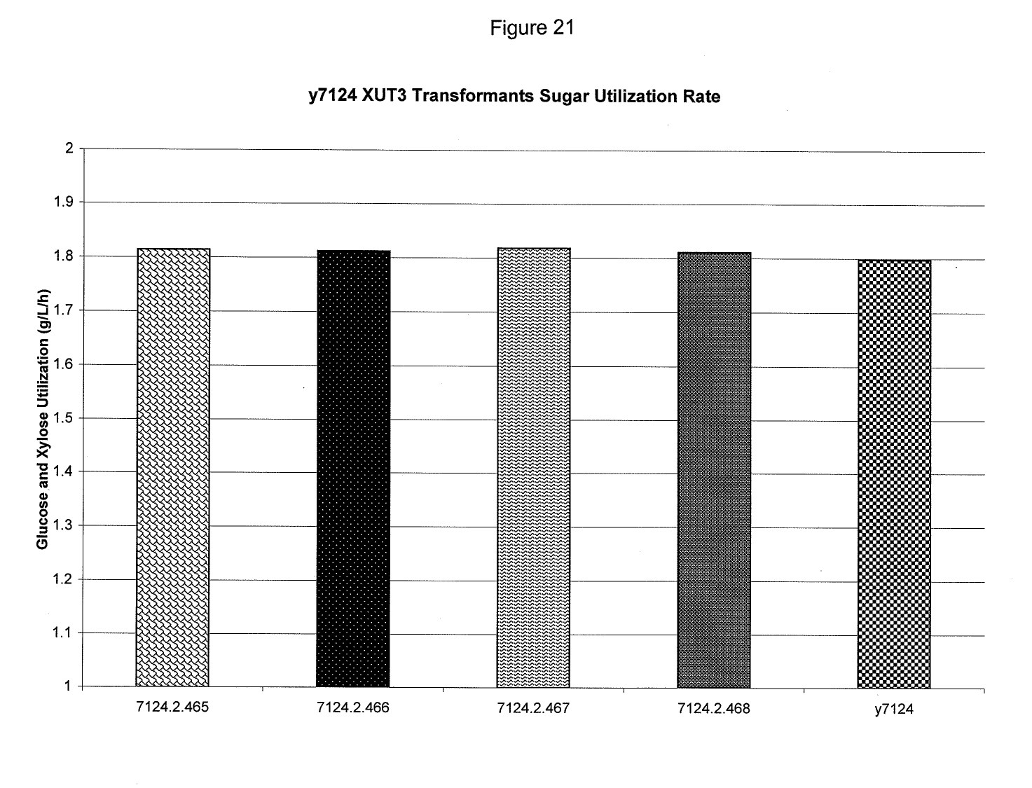

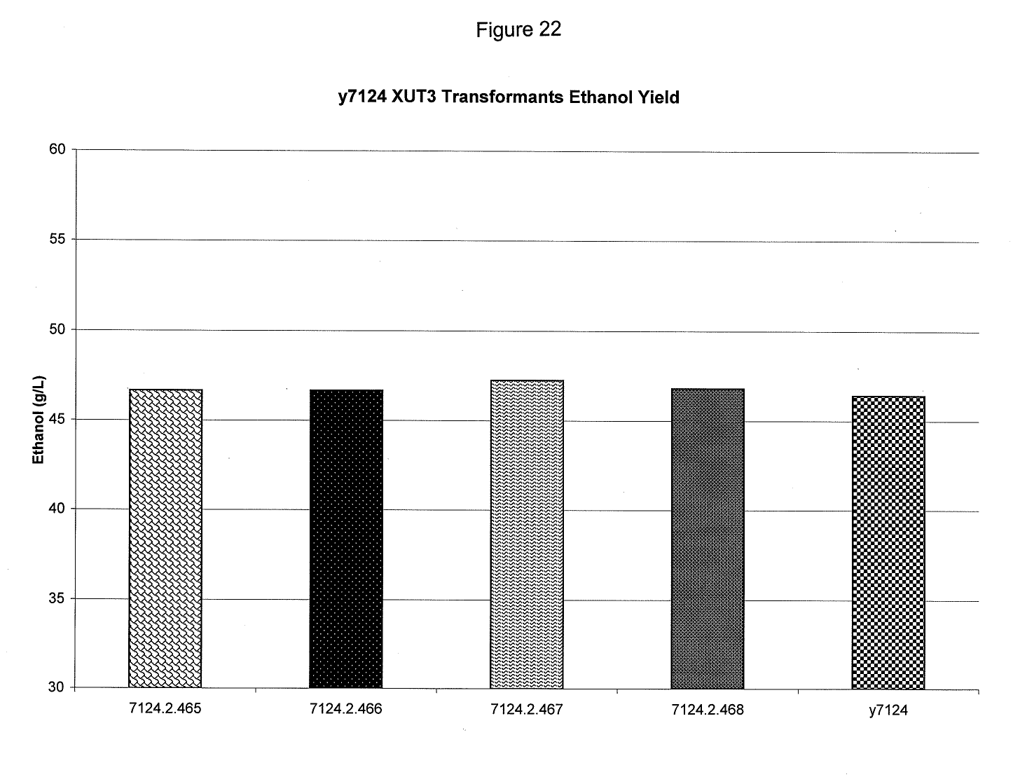

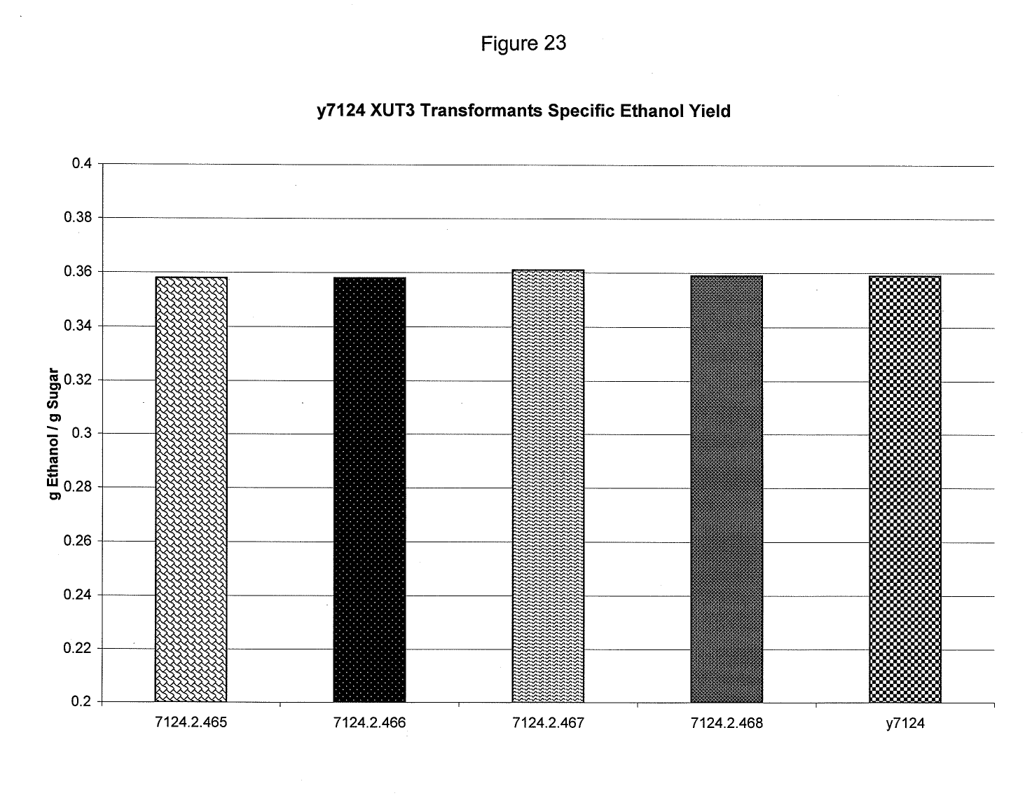

In shake flask, 7124.2.465, 7124.2.466, 7124.2.467, and 7124.2.468 showed no increase in sugar utilization rate, ethanol yield, or specific ethanol yield when compared to the parental y7124 (FIGS. 21-23).

pSDM22 was constructed to contain the P. stipitis HXT4 gene under control of the constitutive P. stipitis TDH3 promoter and the native HXT4 terminator. Approximately 100 μg of plasmid was linearized using the restriction enzymes SacII and KpnI, ethanol precipitated, resuspended in water, creating a fragment that could be directly inserted into the P. stipitis genome. The digested construct was then transformed using a LiAc protocol (2) into NRRL Y-7124, creating 7124.2.471 and 7124.2.472, into a pool of 7124.2.345, 7124.2.346, 7124.2.347, 7124.2.348, 7124.2.349, 7124.2.350, 7124.2.351, 7124.2.352, 7124.2.353, and 7124.2.354 creating 7124.2.446, 7124.2.447, and 7124.2.448, into 7124.1.144 creating 7124.1.170, 7124.1.171, 7124.1.172, 7124.1.173, 7124.1.174, and 7124.1.175, and into a pool of 7124.2.405, 7124.2.406, 7124.2.407, 7124.2.408, and 7124.2.409 creating 7124.2.449, 7124.2.450, 7124.2.451, 7124.2.452, 7124.2.453, and 7124.2.454.

pSDM30 was constructed to contain a synthetic polynucleotide encoding the P. stipitis SUT4 protein under control of the constitutive P. stipitis TDH3 promoter and the native SUT4 terminator, and a synthetic polynucleotide encoding the Zymomonas mobilis ADH1 protein, fused to the P. stipitis TDH3 promoter and terminator. Approximately 100 μg of plasmid was linearized using the restriction enzyme NotI, ethanol precipitated, resuspended in water, creating a fragment that could be directly inserted into the P. stipitis genome. The digested construct was then transformed using a LiAc protocol (2) into NRRL Y-7124 creating 7124.2.477, 7124.2.478, 7124.2.479, 7124.2.480, and 7124.2.481.

### Cellobiose Work:

pSN₂O₇ was constructed to contain the promoter, coding sequence, and terminator for the P. stipitis HXT2.4 gene. Approximately 100 μg of plasmid was linearized using the restriction enzymes SacII and BsrBI, ethanol precipitated, resuspended in water, creating a fragment that could be directly inserted into the P. stipitis genome. The digested construct was then transformed using a LiAc protocol into UC7, creating UC7.1.101 (2).

pSN212 was constructed to contain the P. stipitis BGL5 gene cluster, including the promoters, coding sequences, and terminators for BGL5, EGC2, and HXT2.4. Approximately 100 μg of plasmid was linearized using the restriction enzymes SacII and BsrBI, ethanol precipitated, resuspended in water, creating a fragment that could be directly inserted into the P. stipitis genome. The digested construct was then transformed using a LiAc protocol into UC7, creating UC7.1.102 (2)

Transformants of each reaction were selected for growth on ScD-Ura plates, which contain 0.62 g/l CSM-Leu-Trp-Ura (Bio 101 Systems) and dextrose (2%). Transformants were picked and grown in ScD-Ura liquid medium. Genomic DNA was extracted and PCR was performed to confirm the integration of the constructs. As a control for these strains, the LoxP_Ura3_LoxP cassette was transformed into UC7 (UC7 control).

Fermentation of UC7.1.101 and UC7.1.102. Cultures were started by inoculating a swath of colonies into 150 ml YPD (2% glucose) and grown overnight. The following morning, triplicate flasks were inoculated to a starting OD600 of 14.0 (≈2.0 g/l dry weight of cells). The fermentation was carried out under oxygen limiting conditions with 50 ml of medium in a 125 ml flask, agitation at 100 RPM, and at 30° C. A modified defined minimal medium was used containing trace metal elements and vitamins, which is based on that described by Verduyn et al. (10). For this fermentation, a starting concentration of 50 g 1¹ cellobiose was used.

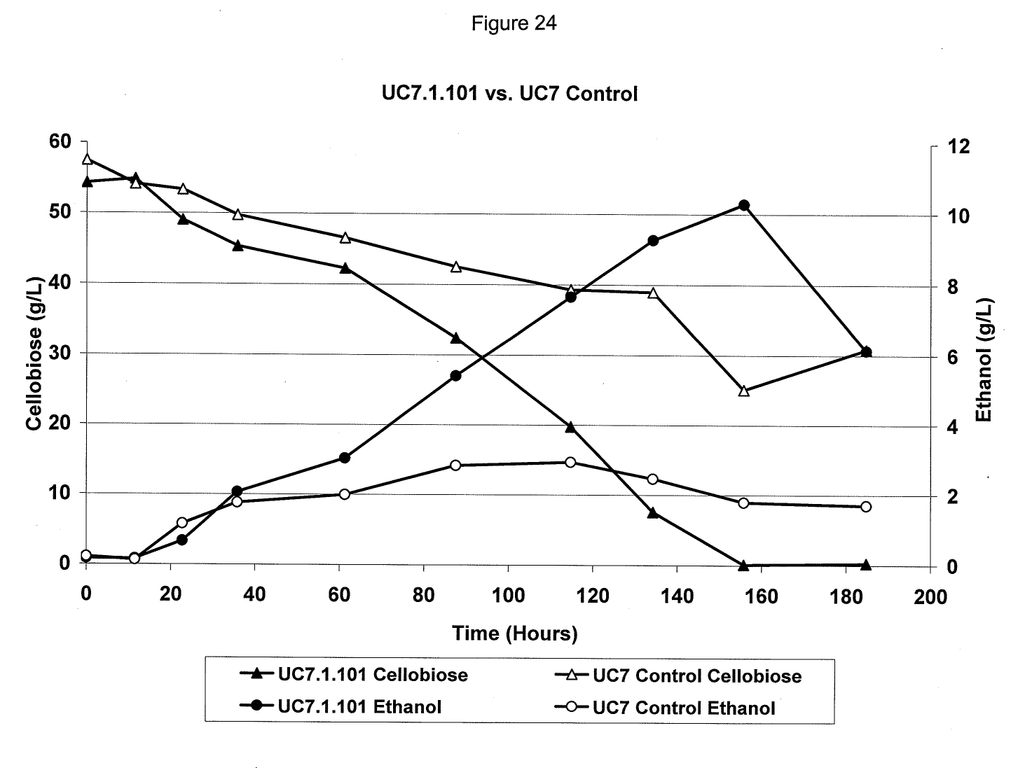

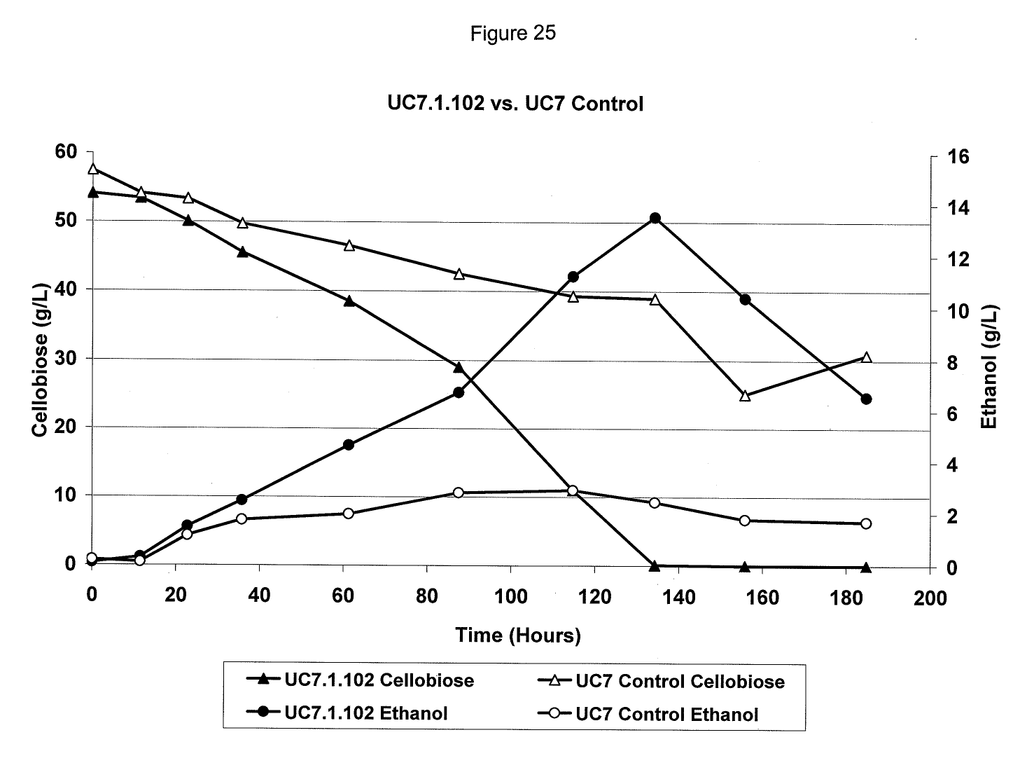

In shake flask trials, both UC7.1.101 and UC7.1.102 were found to use cellobiose at a faster rate than the control UC7 strain. UC7.1.101 had a 100% increase in cellobiose utilization rate (0.322 g/l·h) vs. the control (0.161 g/l·h). UC7.1.102 had a 131.3% increase in cellobiose utilization rate (0.373 g/l·h) vs. the UC7 control (0.161 g/l·h). UC7.1.101 fermented the cellobiose to ethanol with a maximum yield of 10.28 g/l, compared to 2.93 g/l for the control, a 250% increase. The specific ethanol yield increased 75.2% in UC7.1.101 to 0.205 g ethanol/g cellobiose vs. 0.117 g/g for the UC7 control. UC7.1.102 had a maximum ethanol yield of 13.53 g/l, while the UC7 control had a maximum ethanol yield of 2.93 g/l, a 361.8% increase. The specific ethanol yield increased 130.7% in UC7.1.102 to 0.270 g ethanol/g cellobiose vs. 0.117 g/g for the UC7 control (FIGS. 24 and 25).

### Saccharomyces Cellobiose Work:

pSN259 was constructed to contain the P. stipitis BGL5 gene, under the control of the S. cerevisiae TDH3 promoter and terminator, in a 2μ S. cerevisiae vector. Additional S. cerevisiae centromere vectors were constructed to contain P. stipitis genes under control of the S. cerevisiae TDH3 promoter and terminator; pSN260 contains HXT2.4, pSN261 contains HXT2.2, pSN264 contains HXT2.5, and pSN266 contains HXT2.6. Approximately 10 μg of pSN259 along with 10 μg of a either pSN260, pSN261, pSN262, or pSN263 was transformed using a LiAc protocol (Gietz & Woods, 2002, Methods Enzymol 350, 87-98) into S. cerevisiae CEN. PK. 111-27B (Entian K, Kotter P, 2007, 25 Yeast Genetic Strain and Plasmid Collections. In: Methods in Microbiology; Yeast Gene Analysis-Second Edition, Vol. Volume 36 (Ian Stansfield and Michael J R Stark ed), pp 629-666. Academic Press.), creating strains SSN17 (BGL5 and HXT2.4), SSN18 (BGL5 and HXT2.2), SSN21 (BGL5 and HXT2.5), and SSN23 (BGL5 and HXT2.6). A control strain containing empty vectors was also created, SSN7.

Transformants of each reaction were selected for growth on ScD-Trp_Leu plates, which contain 0.62 g/l CSM-Leu-Trp-Ura (Bio 101 Systems) and dextrose (2%). Transformants were picked and grown in ScD-Trp-Leu liquid medium. DNA was extracted and PCR was performed to confirm the presence of the vectors.

Fermentation of SSN17, SSN18, SSN21, and SSN23. Cultures were started by inoculating a swath of colonies into 50 ml of ScD-Trp-Leu and grown overnight. The following morning, triplicate flasks were inoculated to a starting OD600 of 0.5 (≈0.07 g/l dry weight of cells). The fermentation was carried out under oxygen limiting conditions with 50 ml of medium in a 125 ml flask, agitation at 100 RPM, and at 30° C. A defined minimal medium was used containing trace metal elements and vitamins, which is based on that described by Verduyn et al. (10) For this fermentation, a starting concentration of 50 g/l cellobiose and 20 g/l glucose was used.

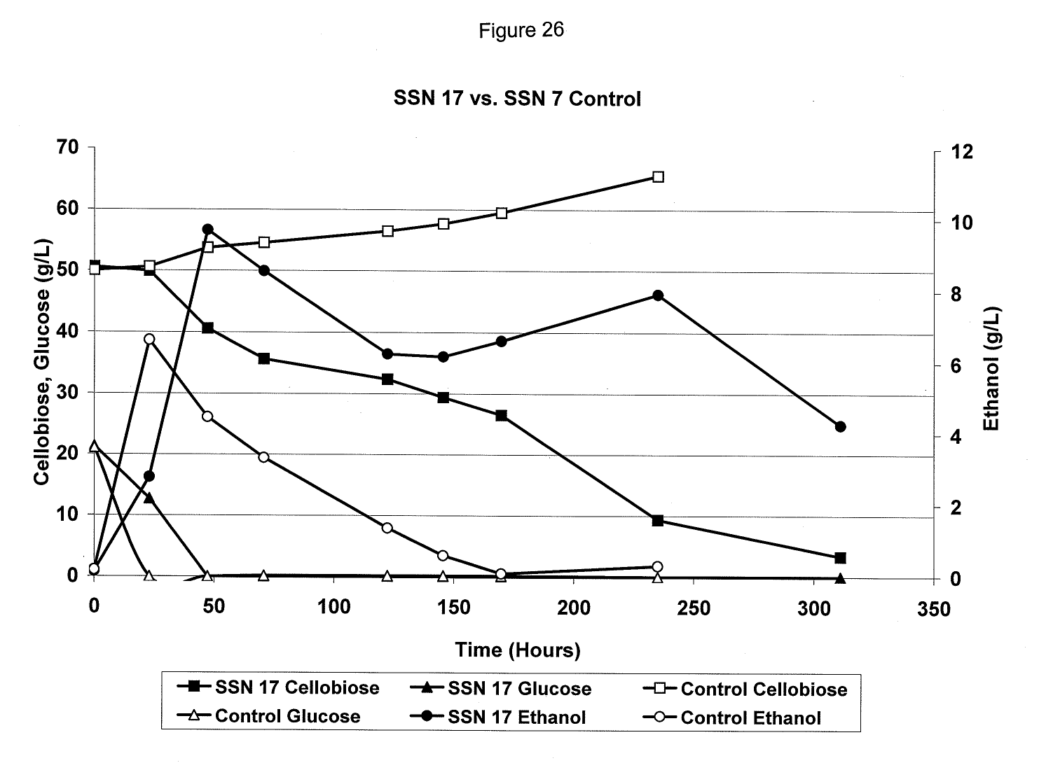

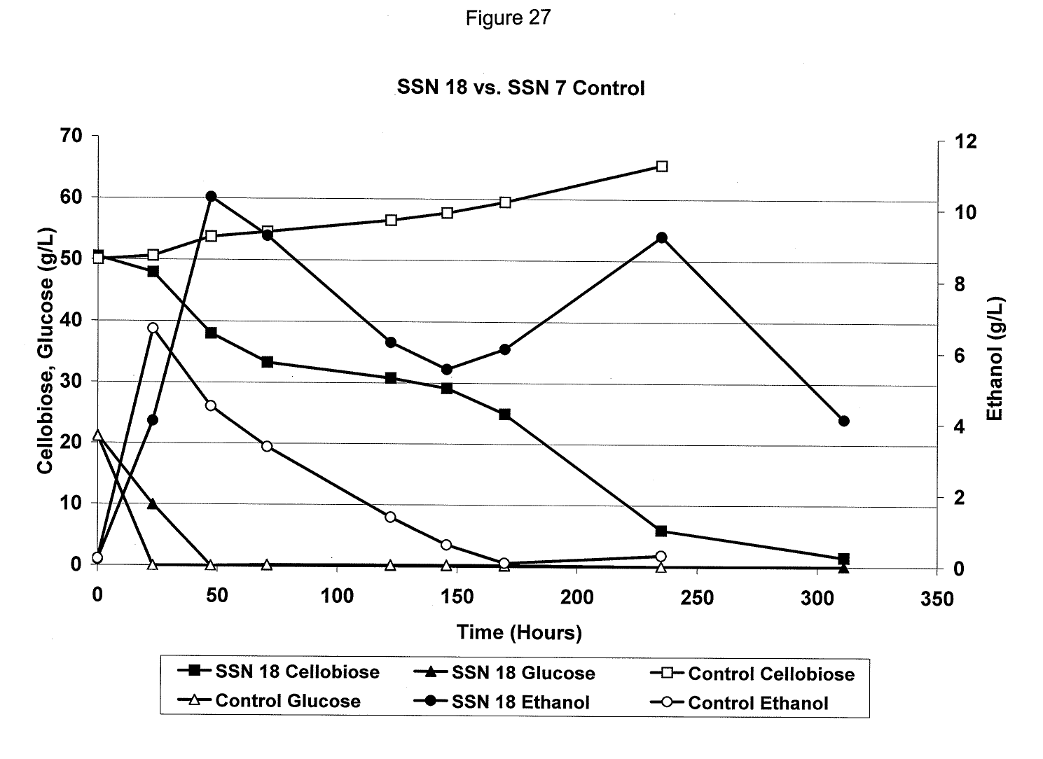

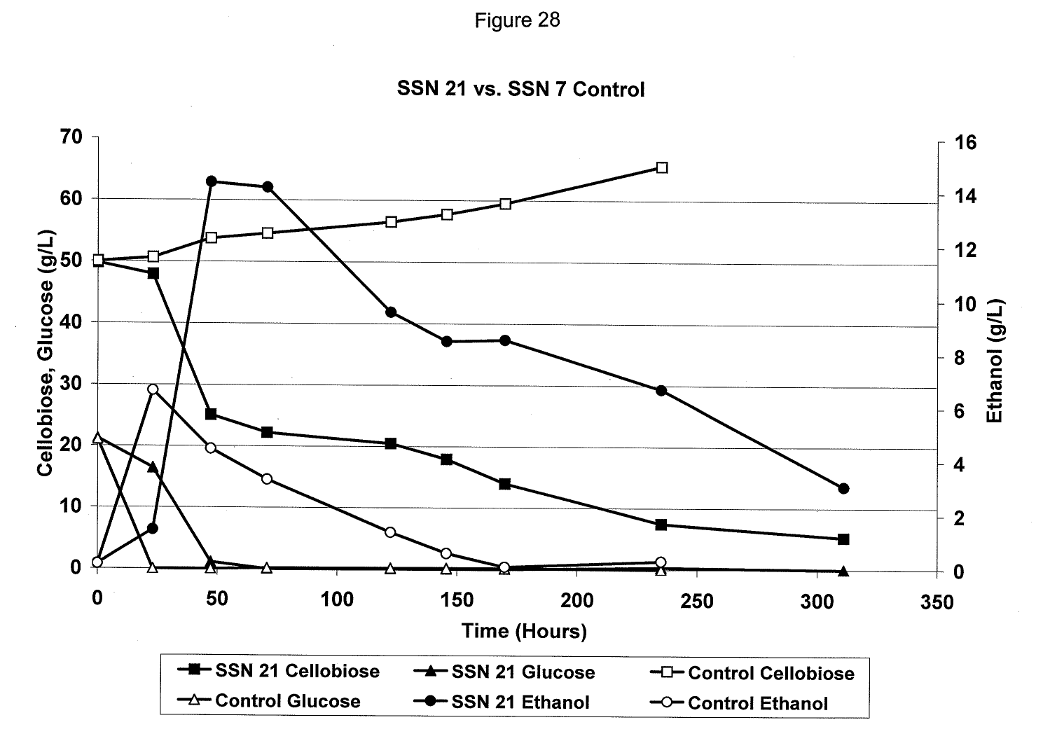

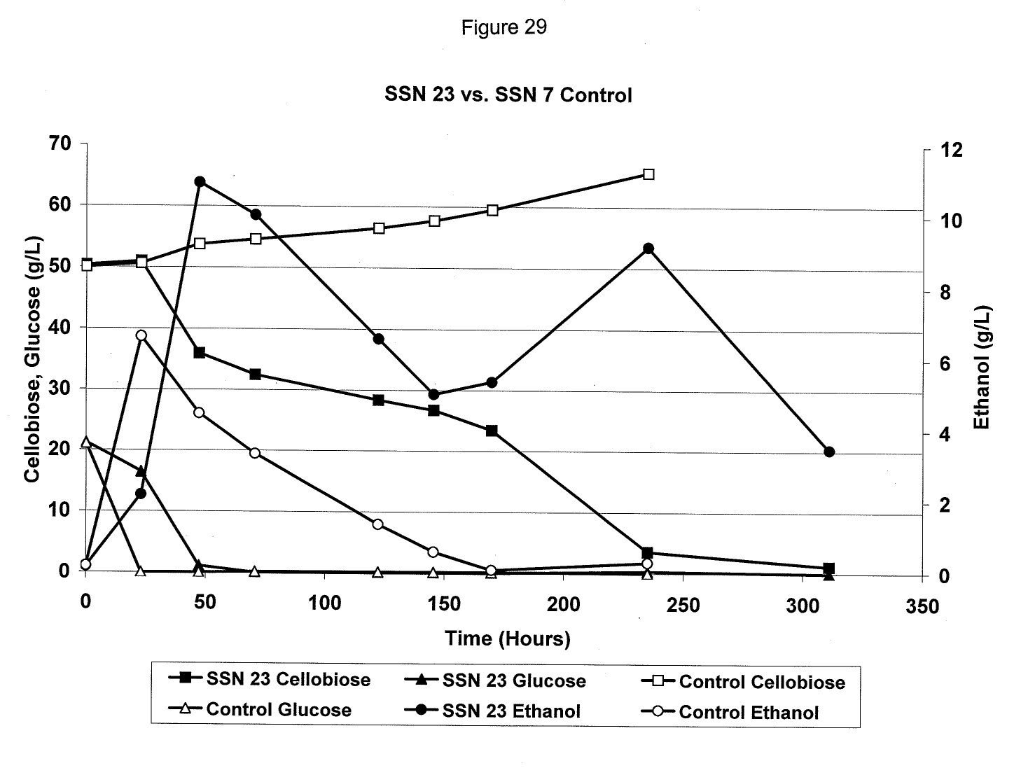

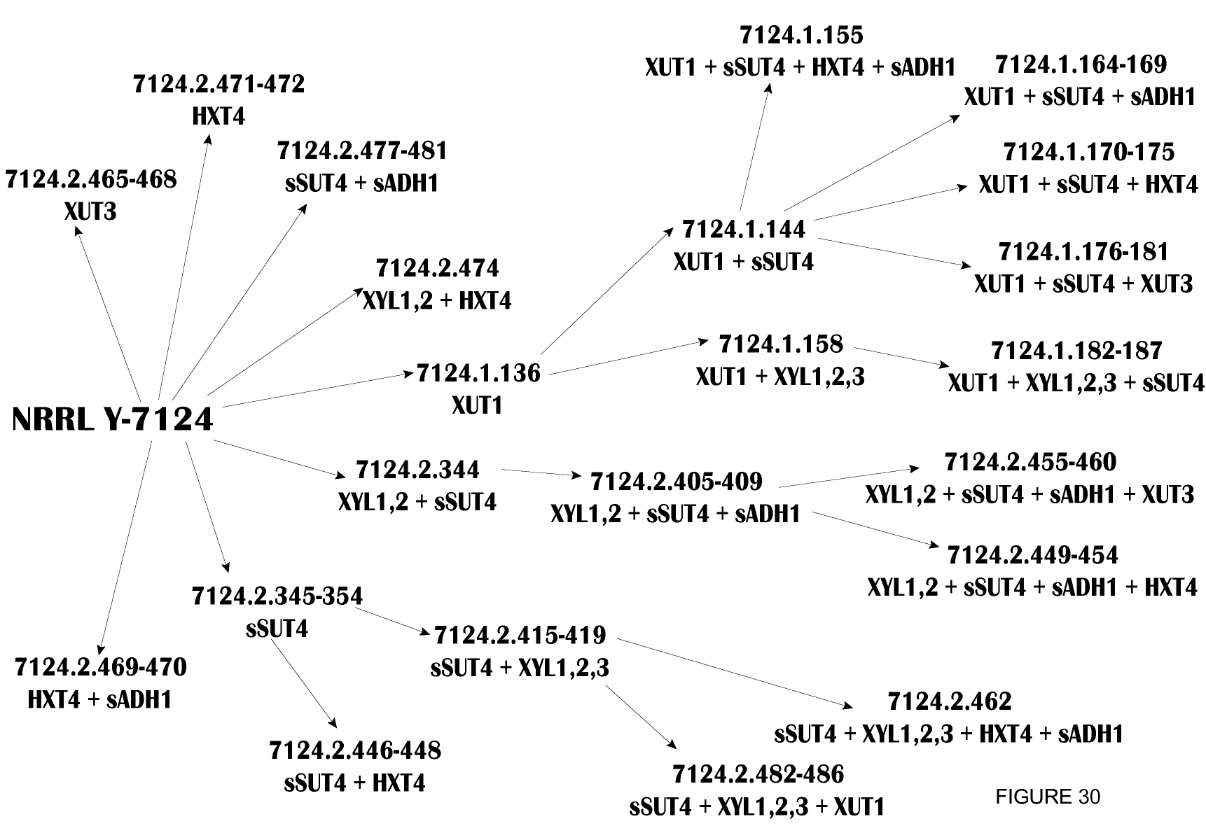

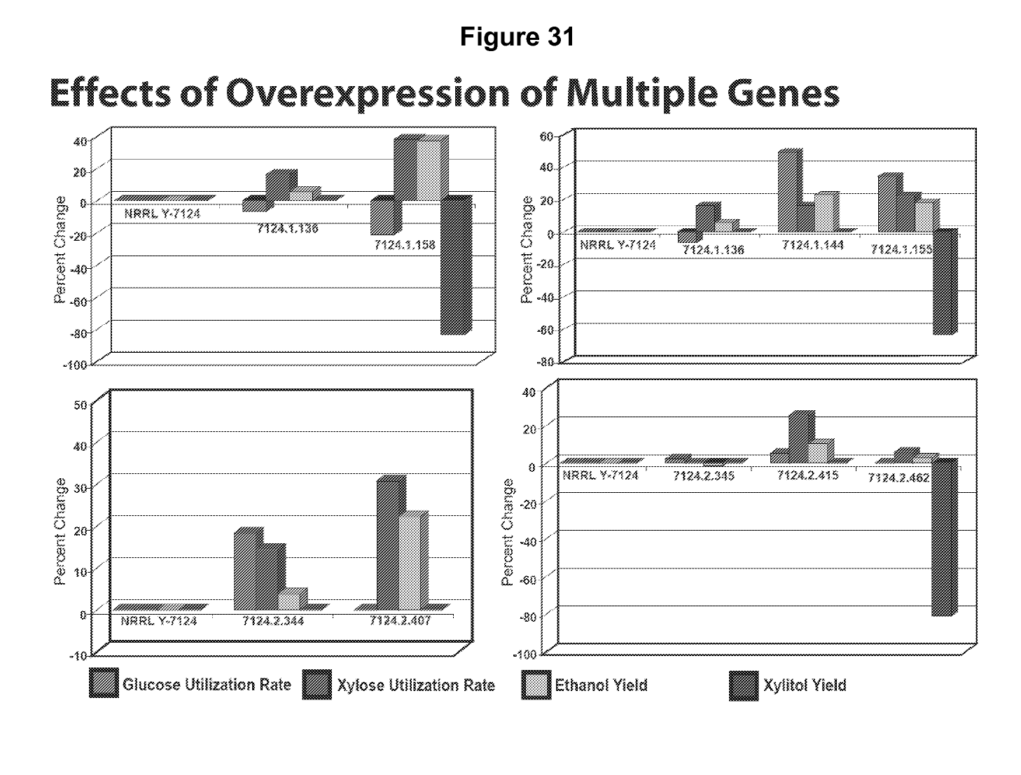

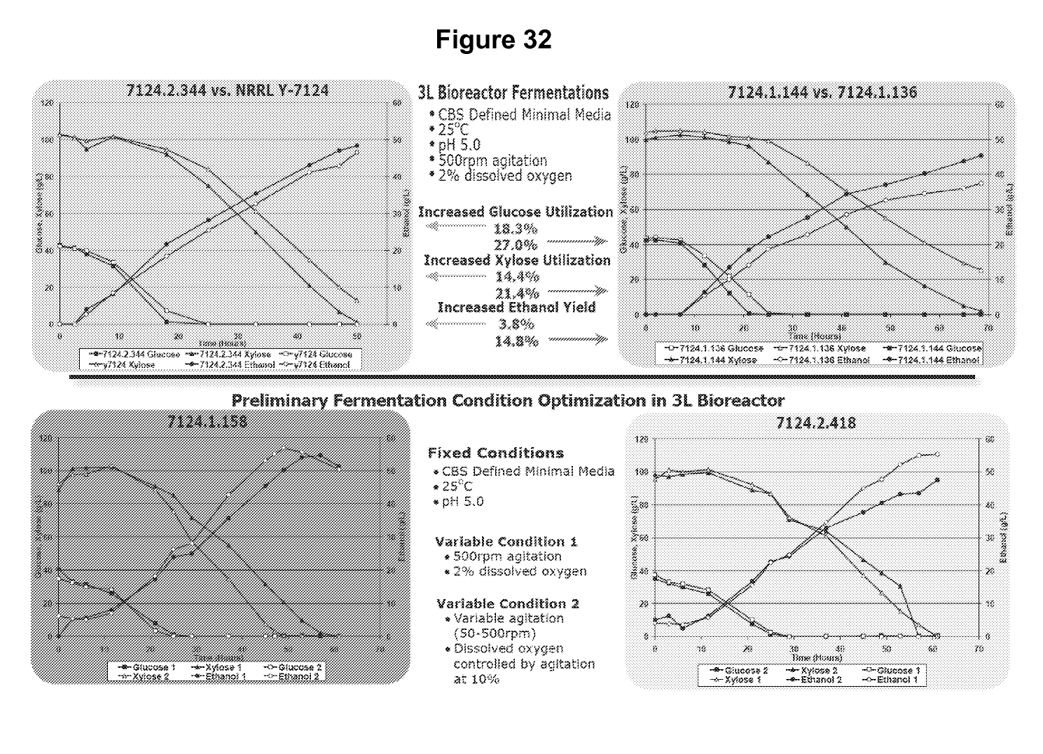

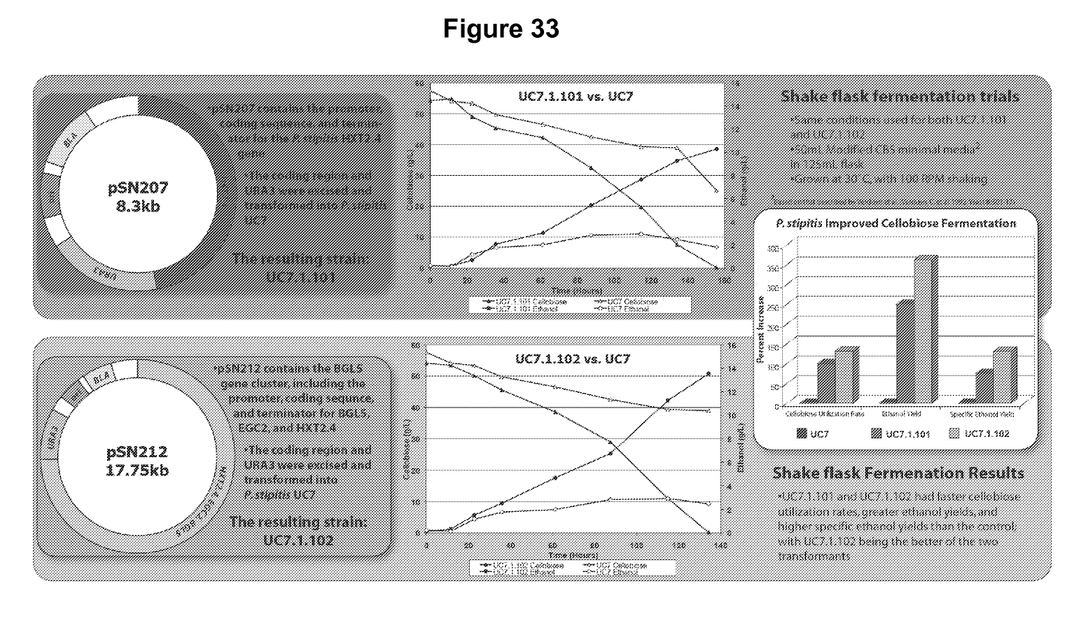

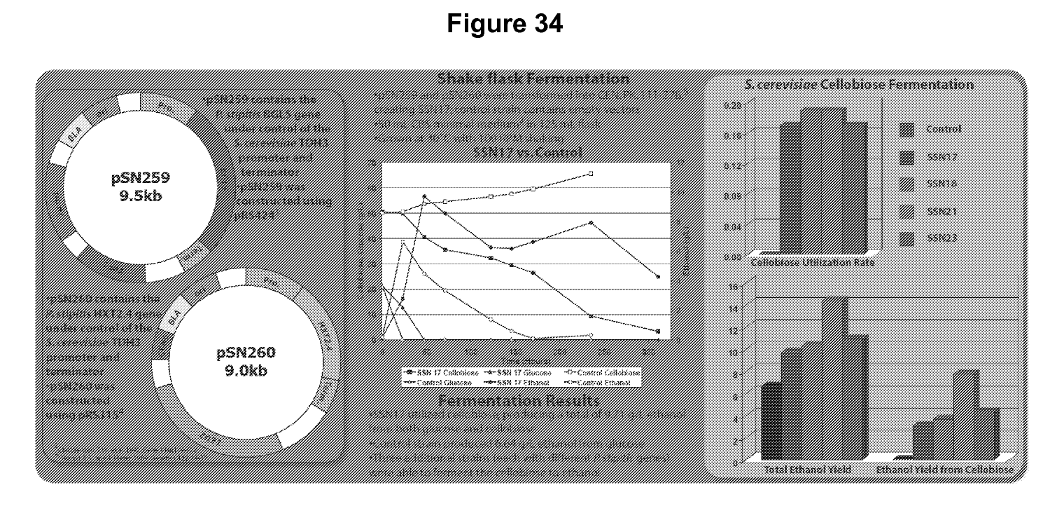

All four transformant strains used the cellobiose, after 238 hours of fermentation, SSN17 had 9.35 g remaining, resulting in a 0.17 g/l·h utilization rate, SSN18 had 5.93 g remaining, resulting in a 0.19 g/l·h utilization rate, SSN21 had 4.77 g remaining, resulting in a 0.19 g/l·h utilization rate, SSN23 had 9.58 g remaining, resulting in a 0.17 g/l·h utilization rate, and the control strain failed to use any of the cellobiose. All four transformants were able to ferment both the glucose and cellobiose to ethanol producing maximum yields of; SSN17 9.71 g/l (3.07 g/l from cellobiose), SSN18 10.31 g/l (3.67 g/l from cellobiose), SSN21 14.37 g/l (7.73 g/l from cellobiose), SSN23 10.93 g/l (4.29 g/l from cellobiose). The control strain was only able to ferment the glucose, producing a maximum yield of 6.64 g/l ethanol (FIGS. 26-29).

Recombineering is a promising in vivo multi-gene cloning method for organisms, such as Saccharomyces cerevisiae, that are especially susceptible to DNA repair via homologous recombination because it overcomes several shortcomings with traditional amplification-ligation cloning techniques. Using a previously engineered plasmid containing native xylose-degradation genes from the yeast Pichia stipitis, pSDM20, a new plasmid designated pMA300.4.3 was genetically recombineered to harbor two additional Pichia stipitis genes, transketolase and transaldolase, and thereby improve Saccharomyces cerevisiae's fermentative capabilities on xylose by increasing activity within the pentose phosphate pathway. Recombineering within Saccharomyces cerevisiae was especially beneficial because it was time-efficient and gave successful in vivo plasmid construction when there were a limited number of restriction enzyme digest sites available. Thus, recombineering proved to be a stable and effective means of plasmid construction in vivo and genetic manipulation in attempts at improving the fermentative capabilities of Saccharomyces cerevisiae. Such proficient manipulation shows promising capabilities of not only Saccharomyces cerevisiae, but also of recombineering in cellulose and hemicellulose degradation in biofuel production.

### Example 2

### Construction of Strain 7124.2.541

pMA300 was constructed to contain the promoter, coding sequence, and terminator for the P. stipitis TAL1 gene, and the promoter, coding sequence, and terminator for the P. stipitis TKT1 gene. Approximately 100 μg of plasmid was linearized using the restriction enzyme ApaLI, ethanol precipitated, resuspended in water, creating a fragment that could be directly inserted into the P. stipitis genome. The digested construct was then transformed into 7124.2.344 using a LiAc protocol (Gietz & Woods, 2002, Methods Enzymol 350, 87-98), thereby creating 7124.2.541.

Transformants were selected via growth on YPD plates containing 50 μg/ml nourseothricin and dextrose (2%). Colonies were grown overnight in YPD+50 μg/ml nourseothricin liquid medium.

The NAT1 gene was removed by transforming the transformants with approximately 10 μg of pJML545 (Jose M. Laplaza and T. W. Jeffries, U.S. Pat. No. 7,501,275 B2; Laplaza, et. al, 2006, Enzyme & Micro Tech, 38:741-747). Transformants were selected on YPD plates containing 50 μg/ml zeocin and dextrose (2%). Colonies were patched onto YPD and YPD+nourseothricin plates to confirm excision of the NAT1 marker.

### Shake Flask Fermentation Assessment of 7124.2.541.

Cultures were started by inoculating a swath of colonies into 50 ml medium in a 125 ml flask and grown overnight at 30° C. and 200 rpm. A modified defined minimal medium containing trace metal elements and vitamins was used (modified from Verduyn et al., 1992, Yeast 8:501-517). It had the following composition: 3.6 g urea 1⁻¹, 14.4 g KH₂PO₄ 1⁻¹, 0.5 g MgSO₄.7H₂O 1⁻¹, 2 ml trace metal solution 1⁻¹, 1 ml vitamin solution 1⁻¹, 500 μl antifoam 289 (Sigma A-8436) 1⁻¹, 10% xylose, 4% glucose. The following morning, triplicate flasks were inoculated to a starting OD₆₀₀ of 4.5 (≈0.7 g/l dry weight of cells) without spinning or washing the cells. The fermentation was carried out under oxygen limiting conditions with 50 ml of medium in a 125 ml flask, agitation at 100 rpm, and at 30° C. Modified defined minimal fermentation medium containing 40 g 1⁻¹ glucose and 100 g 1⁻¹ xylose was used for the fermentation.

In 68 hours strain 7124.2.541 fermented a mixture of glucose and xylose to ethanol at a final concentration of 42.62 g/l, compared to a concentration of 34.26 g/l attained by the parental strain Y-7124 resulting in a 24% increase in final ethanol concentration.

This experiment showed that engineering the overexpression of P. stipitis TAL1 and/or TKT1 in P. stipitis could substantially improve fermentation performance.

### Example 3

### Construction of Strains 7124.2.535 Through 7124.2.539

Strains 7124.2.535 through 7124.2.539 were created by transforming 7124.2.418 with digested pSDM29. pSDM29 was constructed to contain the P. stipitis TDH3 promoter, sSUT4 coding sequence, and P. stipitis SUT4 terminator. Approximately 100 μg of plasmid was linearized using the restriction enzymes NotI and KpnI, ethanol precipitated, resuspended in water, creating a fragment that could be directly inserted into the P. stipitis genome. The digested construct was then transformed into 7124.2.418 using a LiAc protocol (Gietz & Woods, 2002, Methods Enzymol 350, 87-98), thereby creating 7124.2.535 and 7124.2.538.

Transformants were selected via growth on YPD plates containing 50 μg/ml nourseothricin and dextrose (2%). Colonies were grown overnight in YPD+50 μg/ml nourseothricin liquid medium.

The NAT1 gene was removed by transforming the transformants with approximately 10 μg of pJML545 (Jose M. Laplaza and T. W. Jeffries, U.S. Pat. No. 7,501,275 B2; Laplaza, et. al, 2006, Enzyme & Micro Tech, 38:741-747). Transformants were selected on YPD plates containing 50 μg/ml zeocin and dextrose (2%). Colonies were patched onto YPD and YPD+nourseothricin plates to confirm excision of the NAT1 marker.

### Shake Flask Fermentation of Strains 7124.2.535 Through 7124.2.539 in Defined Minimal Medium Containing Hydrolysate.

Cultures were started by inoculating a swath of colonies into 50 ml YPX (4% xylose)+14.6% (v/v, for a total acetic acid concentration of 0.1%) filtered industrial corn stover hydrolysate (provided by EdeniQ, Inc.) in a 125 ml flask and grown for 48 hours at 30° C. and 200 rpm. The following morning, triplicate flasks were inoculated to a starting OD₆₀₀ of 8.0 (≈1.2 g/l dry weight of cells). The fermentation was carried out under oxygen limiting conditions with 50 ml of medium in a 125 ml flask, agitation at 100 rpm, and at 30° C. A modified defined minimal medium was used containing trace metal elements and vitamins, which is based on that described by Verduyn et al. (Verduyn et al., 1992, Yeast 8:501-517). It had the following composition: 3.6 g urea 1⁻¹, 14.4 g KH₂PO₄ 1⁻¹, 0.5 g MgSO₄.7H₂O 1⁻¹, 2 ml trace metal solution 1⁻¹, 1 ml vitamin solution 1⁻¹, 500 μl antifoam 289 (Sigma A-8436) 1⁻¹, 10 ppm Lactrol® (PhibroChem, Ridgefield Park, N.J.), 10 ppm Allpen™ (Alltech, Nicholasville, Ky.), 14.6% (v/v, for a total acetic acid concentration of 0.1%) filtered industrial corn stover hydrolysate (provided by EdeniQ, Inc.), 60 g 1⁻¹ xylose.

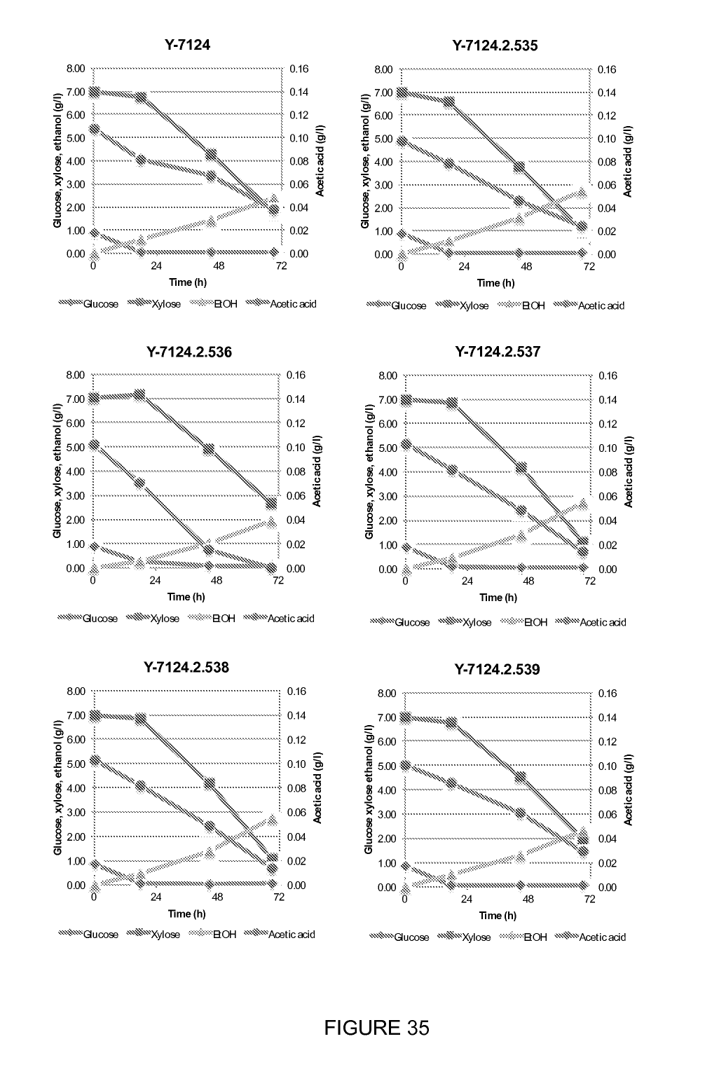

Following incubation and analysis of samples, the relative performance characteristics of several transformants were assessed. Notably, all but one of the transformants showed higher rates of xylose fermentation than Y-7124 and several showed improved rates of acetic acid removal. Strain 7124.2.536 showed markedly increased acetic acid removal but somewhat lower ethanol production and xylose utilization (FIG. 35).

Strain 7124.2.535 was able to ferment xylose in the presence of acetic acid in medium containing industrial corn stover hydrolysate with a final ethanol yield of 27.21 g/l, compared to 23.85 g/l by the parental strain Y-7124 in 69 hours resulting in a 14.08% increase in final ethanol yield. 7124.2.535 consumed 59.62 g/l xylose in 69 hours compared to 52.42 g/l xylose by the parental strain Y-7124 resulting in a 13.7% increase in xylose utilization.

Strain 7124.2.538 was able to ferment xylose in the presence of acetic acid in medium containing industrial corn stover hydrolysate with a final ethanol yield of 27.45 g/l, compared to 23.85 g/l by the parental strain Y-7124 in 69 hours resulting in a 15.1% increase in final ethanol yield. 7124.2.538 consumed 58.84 g/l xylose in 69 hours compared to 52.42 g/l xylose by the parental strain Y-7124 resulting in a 12.24% increase in xylose utilization. 7124.2.538 had a specific yield of 0.466 g ethanol produced/g sugar used, compared to a yield of 0.454 g/g for the parental strain, a 2.6% increase.

This experiment demonstrated that overexpression of a synthetic copy of SUT4 (sSUT4) could substantially improve fermentation performance and that independent clones exhibit various performance characteristics. Multiple transformations and screenings are therefore useful in obtaining improved strains.

### Shake Flask Fermentation of 7124.2.535 in Hydrolysate Containing 0.85 g/l Acetic Acid.

Cultures were started by inoculating a swath of colonies into 50 ml YPX (4% xylose) in a 125 ml flask and grown for 48 hours at 30° C. and 200 rpm. The following morning, triplicate flasks were inoculated to a starting OD₆₀₀ of 9.0 (≈1.35 g/l dry weight of cells). The fermentation was carried out under oxygen limiting conditions with 50 ml of medium in a 125 ml flask, agitation at 100 rpm, and at 30° C. A modified defined minimal medium was used containing trace metal elements and vitamins, which is based on that described by Verduyn et al. (Verduyn et al., 1992, Yeast 8:501-517) and containing filtered industrial corn stover hydrolysate (EdeniQ, Inc.). It had the following composition: 16.6% (v/v, for a final acetic acid concentration of 0.085%) filtered industrial corn stover hydrolysate, 2 ml trace metal solution 1⁻¹, 1 ml vitamin solution 1⁻¹, 10 ppm Lactrol®, 10 ppm Allpen™, 60 g 1⁻¹ xylose, pH 5.0.

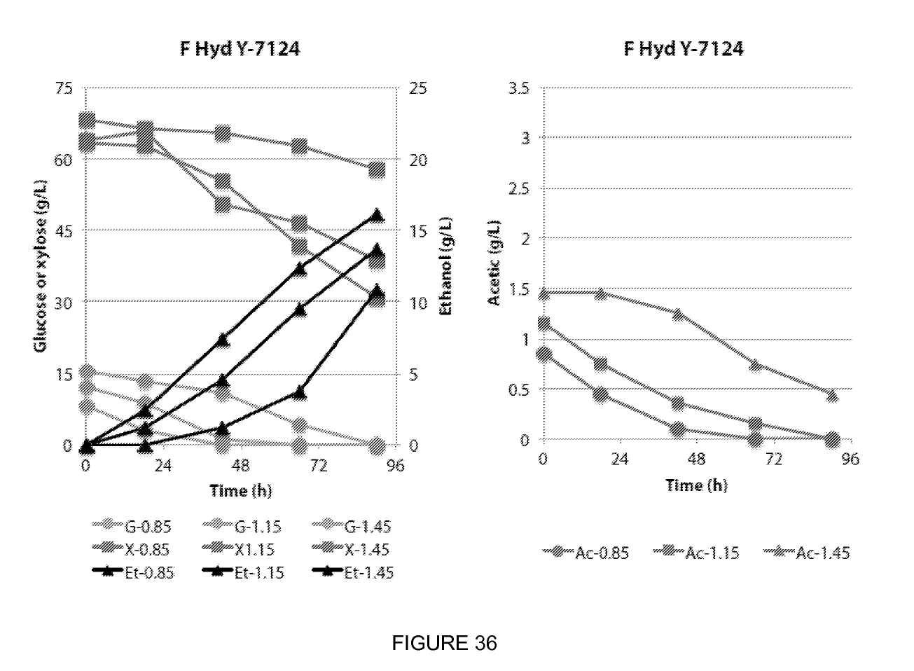

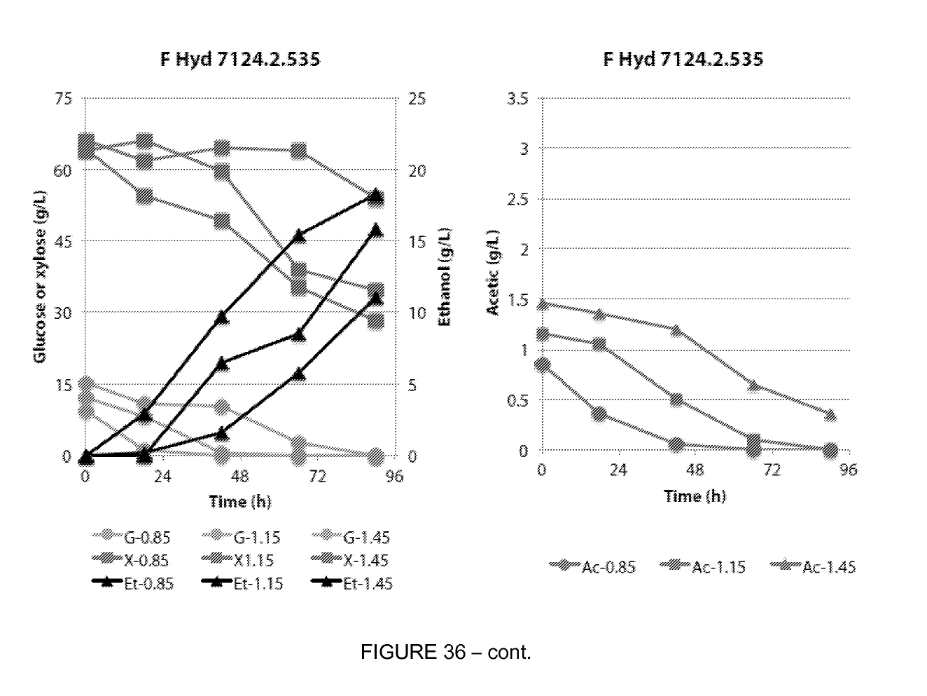

Strain 7124.2.535 was able to ferment xylose in the presence of acetic acid in medium containing industrial corn stover hydrolysate with a final ethanol yield of 18.3 g/l, compared to 16.15 g/l by the parental strain Y-7124 in 90 hours resulting in a 13.3% increase in final ethanol yield. 7124.2.535 consumed 36.0 g/l xylose in 90 hours compared to 27.5 g/l xylose by the parental strain Y-7124 resulting in a 30.9% increase in xylose utilization. 7124.2.538 had a specific yield of 0.466 g ethanol produced/g sugar used, compared to a yield of 0.454 g/g for the parental strain, a 2.6% increase (FIG. 36).

This experiment demonstrated that strains engineered for improved performance in minimal defined medium also exhibit improved performance in hydrolysate medium.

### Shake Flask Fermentation of 7124.2.535 in Hydrolysate Containing 1.15 g/l Acetic Acid.

Cultures were started by inoculating a swath of colonies into 50 ml YPX (4% xylose) in a 125 ml flask and grown for 48 hours at 30° C. and 200 rpm. The following morning, triplicate flasks were inoculated to a starting OD₆₀₀ of 9.0 (≈1.35 g/l dry weight of cells). The fermentation was carried out under oxygen limiting conditions with 50 ml of medium in a 125 ml flask, agitation at 100 rpm, and at 30° C. A modified defined minimal medium was used containing trace metal elements and vitamins, which is based on that described by Verduyn et al. (Verduyn et al., 1992, Yeast 8:501-517) and containing filtered industrial corn stover hydrolysate (EdeniQ, Inc.). It had the following composition: 22.2% (v/v, for a final acetic acid concentration of 0.115%) filtered industrial corn stover hydrolysate, 2 ml trace metal solution 1⁻¹, 1 ml vitamin solution 1⁻¹, 10 ppm Lactrol®, 10 ppm Allpen™, 60 g 1⁻¹ xylose, pH 5.0.

In 90 hours strain 7124.2.535 was able to ferment xylose in the presence of acetic acid in medium containing industrial corn stover hydrolysate with a final ethanol yield of 15.8 g/l, compared to 13.65 g/l by the parental strain Y-7124. This difference comprised a 15.7% increase in final ethanol yield. Strain 7124.2.535 consumed 29.35 g/l xylose in 90 hours compared to 25.25 g/l xylose by the parental strain Y-7124 resulting in a 16.2% increase in xylose utilization. 7124.2.535 had a specific yield of 0.436 g ethanol produced/g sugar used, compared to a yield of 0.421 g/g for the parental strain, a 3.56% increase.

This experiment demonstrated that strains engineered for improved performance in minimal defined medium also exhibit improved performance in hydrolysate medium even when hydrolysate and acetic acid are present at relatively high levels.

### Shake Flask Fermentation by Strain 7124.2.535 in Hydrolysate Containing 0.85 g/l Acetic Acid.

Cultures were started by inoculating a swath of colonies into 50 ml YPX (4% xylose) in a 125 ml flask and grown for 48 hours at 30° C. and 200 rpm. The following morning, triplicate flasks were inoculated to a starting 0D₆₀₀ of 9.0 (≈1.35 g/l dry weight of cells). The fermentation was carried out under oxygen limiting conditions in 125 ml flasks each containing 50 ml of medium. Cultures were incubated at 30° C. and agitated at 100 rpm. A modified defined minimal medium was used containing trace metal elements and vitamins, which is based on that described by Verduyn et al. (Verduyn et al., 1992, Yeast 8:501-517) and containing unfiltered industrial corn stover hydrolysate (EdeniQ, Inc.). It had the following composition: 16.6% (v/v, for a final acetic acid concentration of 0.085%) unfiltered industrial corn stover hydrolysate, 2 ml trace metal solution 1⁻¹, 1 ml vitamin solution 1⁻¹, 10 ppm Lactrol®, 10 ppm Allpen™, 60 g 1⁻¹ xylose, pH 5.0.

Strain 7124.2.535 was able to ferment xylose in the presence of acetic acid in medium containing industrial corn stover hydrolysate with a final ethanol yield of 21.5 g/l, compared to 16.2 g/l by the parental strain Y-7124 in 138 hours resulting in a 32.7% increase in final ethanol yield. Strain 7124.2.535 consumed 45.75 g/l xylose in 138 hours compared to 35.95 g/l xylose by the parental strain Y-7124 resulting in a 27.2% increase in xylose utilization. Strain 7124.2.535 had a specific yield of 0.417 g ethanol produced/g sugar used, compared to a yield of 0.383 g/g for the parental strain, a 8.87% increase.

### Shake Flask Fermentation of 7124.2.535 in Hydrolysate Containing 1.15 g/l Acetic Acid.

Cultures were started by inoculating a swath of colonies into 50 ml YPX (4% xylose) in a 125 ml flask and grown for 48 hours at 30° C. and 200 rpm. The following morning, triplicate flasks were inoculated to a starting 0D₆₀₀ of 9.0 (≈1.35 g/l dry weight of cells). The fermentation was carried out under oxygen limiting conditions with 50 ml of medium in a 125 ml flask, agitation at 100 rpm, and at 30° C. A modified defined minimal medium was used containing trace metal elements and vitamins, which is based on that described by Verduyn et al. (Verduyn et al., 1992, Yeast 8:501-517) and containing unfiltered industrial corn stover hydrolysate (EdeniQ, Inc.). It had the following composition: 22.2% (v/v, for a final acetic acid concentration of 0.115%) unfiltered industrial corn stover hydrolysate, 2 ml trace metal solution 1⁻¹, 1 ml vitamin solution 1⁻¹, 10 ppm Lactrol®, 10 ppm Allpen™, 60 g 1⁻¹ xylose, pH 5.0.

7124.2.535 was able to ferment xylose in the presence of acetic acid in medium containing industrial corn stover hydrolysate with a final ethanol yield of 15.05 g/l, compared to 7.1 g/l by the parental strain Y-7124 in 114 hours resulting in a 111.9% increase in final ethanol yield. 7124.2.535 consumed 29.55 g/l xylose in 114 hours compared to 12.45 g/l xylose by the parental strain Y-7124 resulting in a 137.3% increase in xylose utilization. 7124.2.535 had a specific yield of 0.367 g ethanol produced/g sugar used, compared to a yield of 0.233 g/g for the parental strain, a 57.5% increase.

### Example 4

### Shake Flask Fermentation of Adapted 7124.2.418 and Adapted 7124.2.535.

Hydrolysates with high concentrations of acetic acid are toxic to yeast cells and adversely affect fermentation performance. The purpose of this experiment was to determine whether fermentation performance of engineered cells would further improve or deteriorate upon serial passage in hydrolysate.

Engineered and parental Y-7124 strains were adapted to industrial corn stover hydrolysate (EdeniQ, Inc.) by serial subculture into increasing concentrations of hydrolysate. Cells were adapted in modified defined minimal medium containing trace metal elements and vitamins, which is based on that described by Verduyn et al. (Verduyn et al., 1992, Yeast 8:501-517) and containing filtered industrial corn stover hydrolysate (EdeniQ, Inc.). It had the following composition: 2 ml trace metal solution 1⁻¹, 1 ml vitamin solution 1⁻¹, 10 ppm Lactrol®, 10 ppm Allpen™, 60 g 1⁻¹ xylose, and varying concentrations of filtered industrial corn stover hydrolysate increasing from 14.6% v/v to 43.8% v/v over a period of 14 days. Adapted cultures were started for shake flask fermentation by inoculating a swath of colonies into 100 ml YPX (6% xylose)+14.6% (v/v, for a total acetic acid concentration of 0.1%) filtered industrial corn stover hydrolysate (provided by EdeniQ, Inc.) in a 300 ml flask and grown for 60 hours at 30° C. and 100 rpm. Triplicate flasks were inoculated to a starting 0D₆₀₀ of 9.0 (≈1.35 g/l dry weight of cells). The fermentation was carried out under oxygen limiting conditions with 50 ml of medium in a 125 ml flask, agitation at 100 rpm, and at 30° C. A modified defined minimal medium was used containing trace metal elements and vitamins, which is based on that described by Verduyn et al. (Verduyn et al., 1992, Yeast 8:501-517) and containing filtered pre-fermented industrial corn stover hydrolysate (EdeniQ, Inc.). It had the following composition: 52.6% (v/v, for a final acetic acid concentration of 0.18%) filtered pre-fermented industrial corn stover hydrolysate, 3.6 g urea 1⁻¹, 14.4 g KH₂PO₄ 1⁻¹, 0.5 g MgSO₄.7H₂O 1⁻¹, 2 ml trace metal solution 1⁻¹, 1 ml vitamin solution 1⁻¹, 10 ppm Lactrol®, 10 ppm Allpen™, 60 g 1⁻¹ xylose.

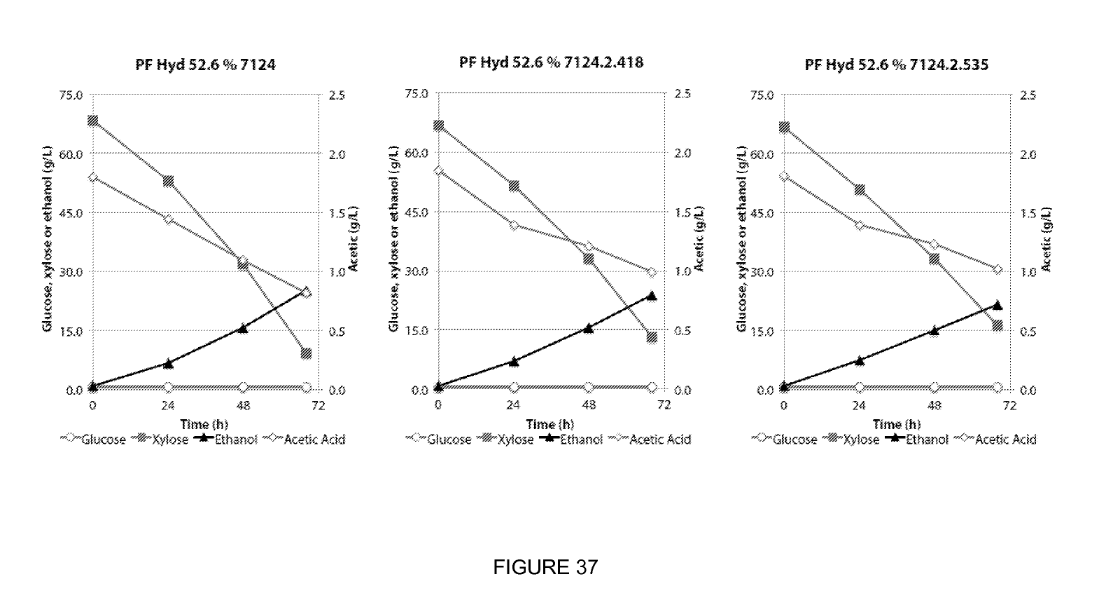

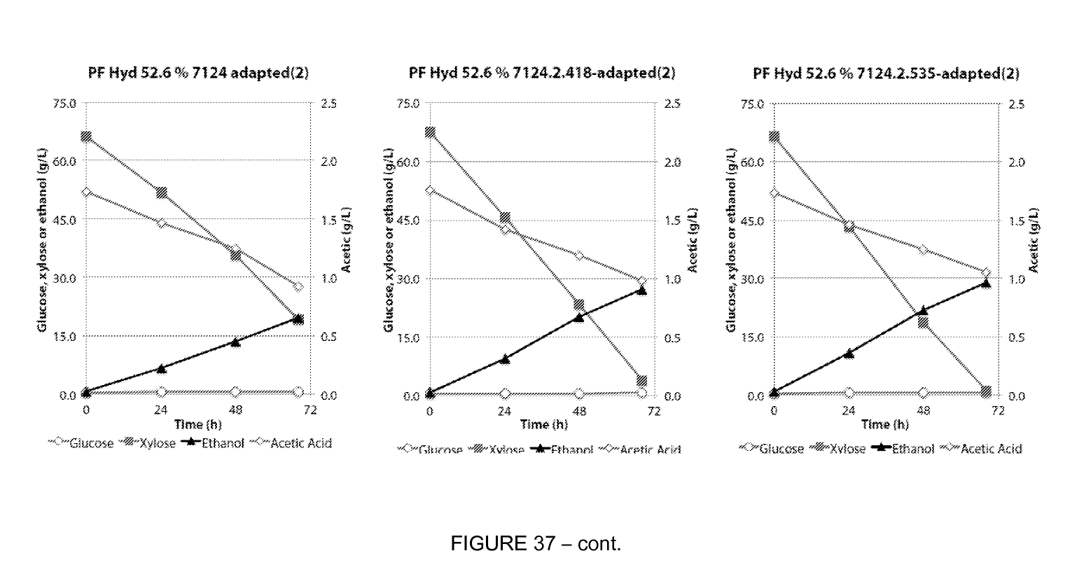

Adapted 7124.2.418 was able to ferment xylose in the presence of acetic acid in medium containing industrial corn stover hydrolysate with a final ethanol yield of 22.18 g/l, compared to 18.25 g/l by the adapted parental strain Y-7124 in 72 hours resulting in a 21.5% increase in final ethanol yield. Adapted 7124.2.418 consumed 52.4 g/l xylose in 72 hours compared to 44.48 g/l xylose by the adapted parental strain Y-7124 resulting in a 17.8% increase in xylose utilization. Adapted 7124.2.418 had a specific yield of 0.415 g ethanol produced/g sugar used, compared to a yield of 0.401 g/g for the adapted parental strain, a 3.49% increase (FIG. 37).

Adapted strain 7124.2.535 was able to ferment xylose in the presence of acetic acid in medium containing industrial corn stover hydrolysate with a final ethanol yield of 23.8 g/l, compared to 18.25 g/l by the adapted parental strain Y-7124 in 72 hours resulting in a 30.4% increase in final ethanol yield. Adapted 7124.2.535 consumed 56.73 g/l xylose in 72 hours compared to 44.48 g/l xylose by the adapted parental strain Y-7124 resulting in a 27.5% increase in xylose utilization. Adapted 7124.2.535 had a specific yield of 0.412 g ethanol produced/g sugar used, compared to a yield of 0.401 g/g for the adapted parental strain, a 2.7% increase.

When comparing adapted strains to non-adapted strains, adapted Y-7124 produced 18.25 g/l ethanol in 72 hours, compared to 17.48 g/l by the non-adapted strain resulting in a 4% increase in final ethanol yield. Adapted 7124.2.418 produced 22.18 g/l ethanol in 72 hours, compared to 18.84 g/l by the non-adapted strain resulting in a 17.7% increase in final ethanol yield. Adapted 7124.2.535 produced 23.8 g/l ethanol in 72 hours, compared to 18.53 g/l by the non-adapted strain resulting in a 28.4% increase in final ethanol yield. Adapted Y-7124 consumed 44.51 g/l xylose in 72 hours, compared to 43.54 g/l by the non-adapted strain resulting in a 2.2% increase in xylose consumption. Adapted 7124.2.418 consumed 52.4 g/l xylose in 72 hours, compared to 45.41 g/l by the non-adapted strain resulting in a 15.4% increase in xylose consumption. Adapted 7124.2.535 consumed 56.73 g/l xylose in 72 hours, compared to 45.26 g/l by the non-adapted strain resulting in a 25.3% increase in xylose consumption.

This experiment showed that adapting the engineered strains to growth in hydrolysate containing acetic acid substantially improves performance relative to the performance of the non-adapted cells.

### Example 5

### Shake Flask Fermentation Assessment of Cell Recycling with Adapted Strains of Adapted 7124.2.418 and Adapted 7124.2.535.

Cell recycling might be used as a means for inoculum propagation on an industrial scale. Therefore, the purpose of this experiment was to determine whether the performance of adapted cells would improve or degenerate upon subsequent recycling of cells from one fermentation trial to another.

Engineered and parental Y-7124 strains were adapted to industrial corn stover hydrolysate (EdeniQ, Inc.) by serial subculture into increasing concentrations of hydrolysate. Cells were adapted in modified defined minimal medium containing trace metal elements and vitamins, which is based on that described by Verduyn et al. (Verduyn et al., 1992, Yeast 8:501-517) and containing filtered industrial corn stover hydrolysate (EdeniQ, Inc.). It had the following composition: 2 ml trace metal solution 1⁻¹, 1 ml vitamin solution 1⁻¹, 10 ppm Lactrol®, 10 ppm Allpen™, 60 g 1⁻¹ xylose, and varying concentrations of filtered industrial corn stover hydrolysate increasing from 14.6% v/v to 43.8% v/v over a period of 14 days. Adapted cultures were started for shake flask fermentation by inoculating a swath of colonies into 100 ml YPX (6% xylose)+14.6% (v/v, for a total acetic acid concentration of 0.1%) filtered industrial corn stover hydrolysate (provided by EdeniQ, Inc.) in a 300 ml flask and grown for 60 hours at 30° C. and 100 rpm. Triplicate flasks were inoculated to a starting 0D₆₀₀ of 9.0 (≈1.35 g/l dry weight of cells). The fermentation was carried out under oxygen limiting conditions with 50 ml of medium in a 125 ml flask, agitation at 100 rpm, and at 30° C. A modified defined minimal medium was used containing trace metal elements and vitamins, which is based on that described by Verduyn et al. (Verduyn et al., 1992, Yeast 8:501-517) and containing filtered pre-fermented industrial corn stover hydrolysate (EdeniQ, Inc.). It had the following composition: 52.6% (v/v, for a final acetic acid concentration of 0.18%) filtered pre-fermented industrial corn stover hydrolysate, 3.6 g urea 1⁻¹, 14.4 g KH₂PO₄ 1⁻¹, 0.5 g MgSO₄.7H₂O 1⁻1, 2 ml trace metal solution 1⁻¹, 1 ml vitamin solution 1⁻¹, 10 ppm Lactrol®, 10 ppm Allpen™, 60 g 1¹ xylose. After a fermentation time of 72 hours, each flask was transferred to a 50 ml conical centrifuge tube and cells were pelleted and resuspended in 3 ml 30% glycerol and stored at −20° C. for 72 hours. Cells were thawed and washed with water and recycled into fresh fermentation flasks. The fermentation of recycled cells was carried out under oxygen limiting conditions with 50 ml of medium in a 125 ml flask, agitation at 100 rpm, and at 30° C. A modified defined minimal medium was used containing trace metal elements and vitamins, which is based on that described by Verduyn et al. (Verduyn et al., 1992, Yeast 8:501-517) and containing filtered pre-fermented industrial corn stover hydrolysate (EdeniQ, Inc.). It had the following composition: 52.6% (v/v, for a final acetic acid concentration of 0.18%) filtered pre-fermented industrial corn stover hydrolysate, 3.6 g urea 1⁻¹, 14.4 g KH₂PO₄ 1¹, 0.5 g MgSO₄.7H₂O 1⁻1, 2 ml trace metal solution 1⁻¹, 1 ml vitamin solution 1⁻¹, 10 ppm Lactrol®, 10 ppm Allpen™, 60 g 1¹ xylose.

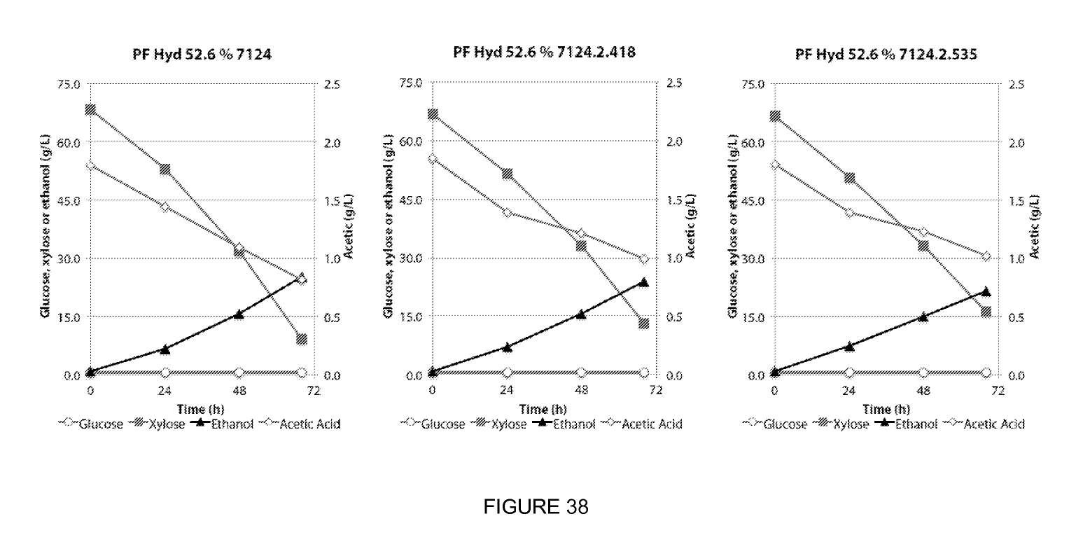

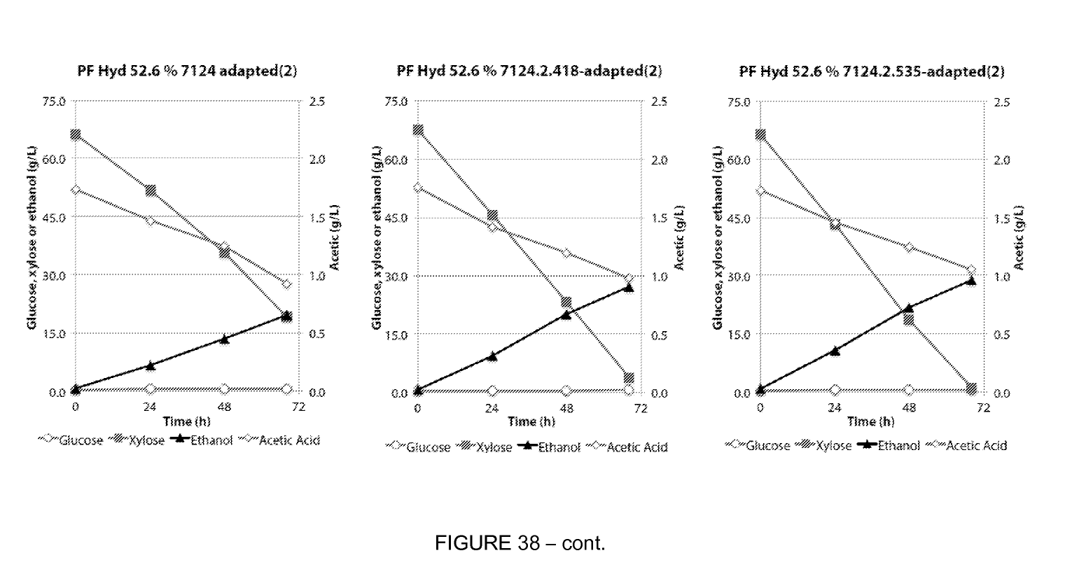

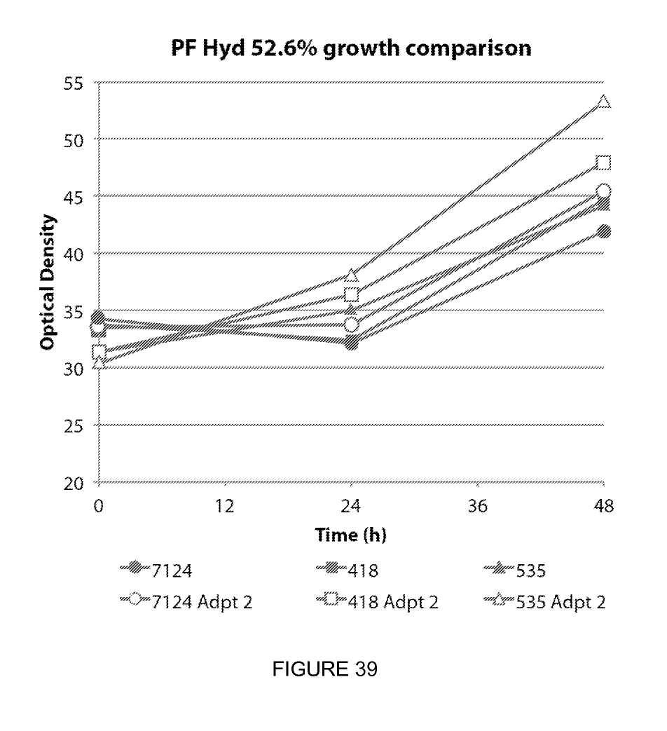

Recycled adapted 7124.2.418 was able to ferment xylose in the presence of acetic acid in medium containing industrial corn stover hydrolysate with a final ethanol yield of 27.23 g/l, compared to 19.75 g/l by the recycled adapted parental strain Y-7124 in 68 hours resulting in a 37.8% increase in final ethanol yield. Recycled adapted 7124.2.418 consumed 63.34 g/l xylose in 68 hours compared to 46.79 g/l xylose by the recycled adapted parental strain Y-7124 resulting in a 35.3% increase in xylose utilization. Recycled adapted 7124.2.418 had a specific yield of 0.429 g ethanol produced/g sugar used, compared to a yield of 0.420 g/g for the recycled adapted parental strain, a 2.1% increase (FIG. 38).

Recycled adapted 7124.2.535 was able to ferment xylose in the presence of acetic acid in medium containing industrial corn stover hydrolysate with a final ethanol yield of 28.86 g/l, compared to 19.75 g/l by the recycled adapted parental strain Y-7124 in 68 hours resulting in a 46% increase in final ethanol yield. Recycled adapted 7124.2.535 consumed 65.24 g/l xylose in 68 hours compared to 46.79 g/l xylose by the recycled adapted parental strain Y-7124 resulting in a 38% increase in xylose utilization. Recycled adapted 7124.2.535 had a specific yield of 0.442 g ethanol produced/g sugar used, compared to a yield of 0.420 g/g for the recycled adapted parental strain, a 5.2% increase.

When comparing recycled adapted strains to recycled non-adapted strains, recycled adapted Y-7124 produced 19.75 g/l ethanol in 68 hours, compared to 25.32 g/l by the recycled non-adapted strain resulting in a 21.9% decrease in final ethanol yield. Recycled adapted 7124.2.418 produced 27.23 g/l ethanol in 68 hours, compared to 23.75 g/l by the recycled non-adapted strain resulting in a 14.7% increase in final ethanol yield. Recycled adapted 7124.2.535 produced 28.86 g/l ethanol in 68 hours, compared to 21.47 g/l by the recycled non-adapted strain resulting in a 34.4% increase in final ethanol yield. Recycled adapted Y-7124 consumed 46.97 g/l xylose in 68 hours, compared to 59.34 g/l by the recycled non-adapted strain resulting in a 20.8% decrease in xylose consumption. Recycled adapted 7124.2.418 consumed 63.34 g/l xylose in 68 hours, compared to 53.76 g/l by the recycled non-adapted strain resulting in a 17.8% increase in xylose consumption. Recycled adapted 7124.2.535 consumed 65.24 g/l xylose in 68 hours, compared to 50.59 g/l by the recycled non-adapted strain resulting in a 28.9% increase in xylose consumption. Recycled adapted Y-7124 had a specific yield of 0.420 g ethanol produced/g sugar used, compared to a yield of 0.426 g/g for the recycled non-adapted strain, a 1.4% decrease. Recycled adapted 7124.2.535 had a specific yield of 0.442 g ethanol produced/g sugar used, compared to a yield of 0.424 g/g for the recycled non-adapted strain, a 4.2% increase.

This experiment showed that recycling cells that had been engineered for improved fermentation and subsequently adapted to hydrolysate could further improve fermentation performance, thereby enabling a convenient method for cell propagation on an industrial scale.

### Example 6

### Further Improvement of Fermentation Performance by Mating Independent Strains and Transformants of Pichia stipitis

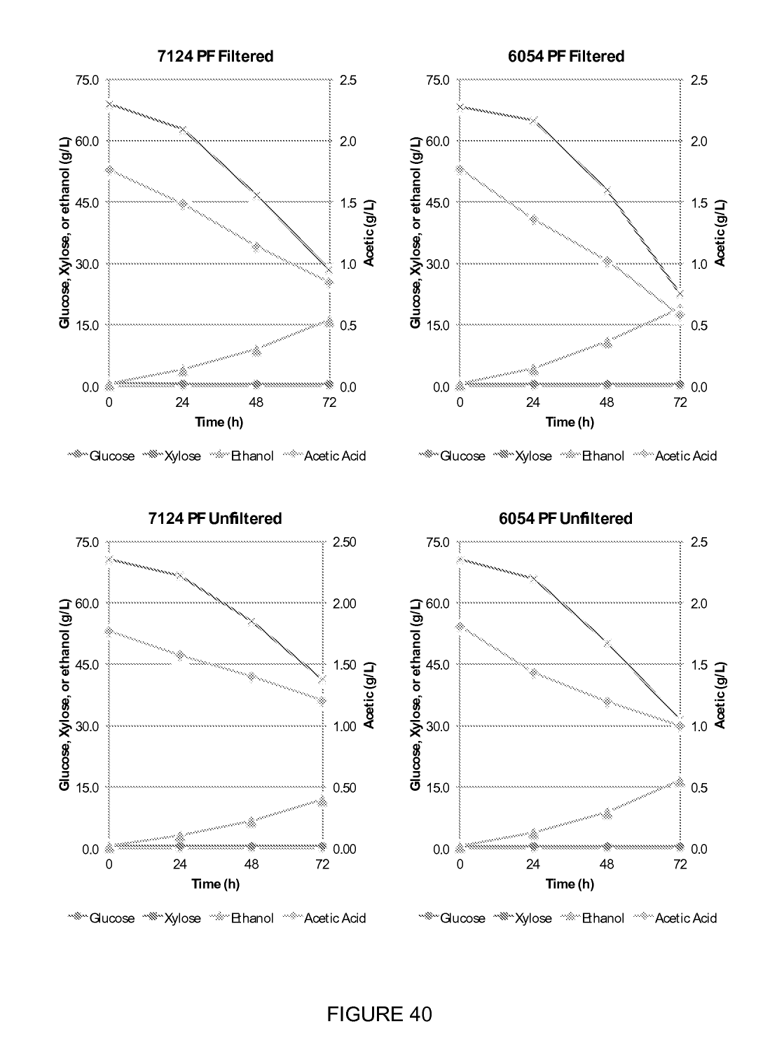

The objective of this experiment was to determine if additional performance improvement could be realized by mating strains of Pichia stipitis that had been obtained through completely independent lines of transformation and selection. The native Scheffersomyces (Pichia) stipitis strains CBS 6054 and NRRL Y-7124 were independently isolated and characterized. Genomic sequencing of these two strains reveals more than 42 thousand single nucleotide variants (SNVs), which are essentially equivalent to single nucleotide polymorphisms (SNPs) and 3 thousand insertions or deletions (indels) when compared to one another: See world wide web at genome.jgi-psf.org/Picst3/Picst3.home.html. Other studies have shown substantial differences between these two strains in their abilities to ferment cellobiose and in their capacities to ferment hydrolysates (FIG. 40).

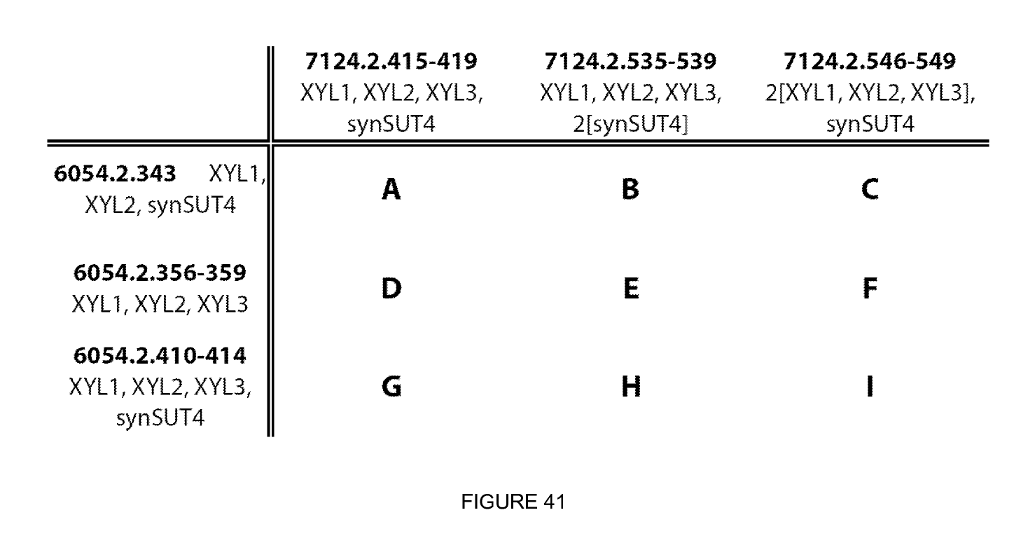

It was unknown whether different lines of independently derived S. stipitis transformants could be mated and whether selection for resistance to hydrolysate would obtain improved performance. Nine different crosses of independently derived lines of cells were made (FIG. 41).

Independent transformants of CBS 6054 were created by transforming the parental strain with expression cassetts described previously and the resulting transformants, 6054.2.343 (XYL1, XYL2, SynSUT4); 6054.2.356-359 (XYL1, XYL2, XYL3) and 6054.2.410-414 (XYL1, XYL2, XYL3, synSUT4), were employed in mating trials with transformants of NRRL Y-7124.

Cells from six engineered strains of Scheffersomyces stipitis (three strains derived from CBS 6054 and three strains derived from NRRL Y-7124) were mated by pairwise mixing of the cells on the surface of a SporB plate, which contained 1.7 g/l Yeast Nitrogen Base (without amino acids or ammonium sulfate), 0.05 g/l ammonium sulfate, 1.0 g/l xylose and 1.0 g/l cellobiose in 3% agar. For example a SporB plate, which contained 1.7 g/l Yeast Nitrogen Base (without amino acids or ammonium sulfate), 0.05 g/l ammonium sulfate, 1.0 g/l xylose and 1.0 g/l cellobiose in 3% agar. The inoculated plates were incubated at 30° C. for 21 days. For example, 6054.2.343 was crossed in pairwise fashions with pooled transformants 7124.2.415 to 419, 7124.2.535 to 539 or 7124.2.546 to 549 to create the mated hybrids A, B and C, respectively. Six other crosses were carried out in a similar manner according to the design depicted in FIG. 41. The inoculated plates were then incubated at 30° C. for 21 days.

During this time, samples of cells were removed from the plate and examined microscopically. The cells were observed to form mating figures and spore bodies. A swath of cells from the sporB plate was inoculated into 50 ml of YPX (2% xylose) in a 125 ml flask and incubated for 8 hours at 30° C. for 8 hours to recover sporulated cells. Following this initial growth period, hydrolysate was added to the growing culture of YPX sufficient to increase the acetic acid content of the medium to approximately 0.3%. Notably, crosses (A) and (I) did not show viable cells following introduction of hydrolysate. Media from those inoculated cultures that did not grow out were serially transferred as negative controls throughout the subsequent adaptation.

Once cells had grown out from the first addition of hydrolysate (cultures B through H), the strains were adapted to industrial corn stover hydrolysate containing inhibitory concentrations of acetic acid (EdeniQ) by serial subculture into increasing concentrations of hydrolysate ranging from 33% v/v (0.2% acetic acid) hydrolysate to 97.5% v/v (0.35% acetic acid) hydrolysate over a period of 14 days. Strains were then maintained in 87.5% v/v (0.3% acetic acid) hydrolysate for 33 days by serial subculture every 4-7 days, and then adapted to 87.5% v/v (0.5% acetic acid) harsh hydrolysate over 24 days via serial subculture every 4-7 days.

When the resulting crosses were examined microscopically they showed substantial differences in morphology and culture characteristics. Some strains predominantly formed cells that were yeast-like in appearance while other strains predominantly formed pseudomycelial cells. Some strains tended to form pellets which rapidly sank to the bottom of the flask. Other strains remained in suspension. Strains also showed notable differences in colonial morphology when plated onto agar medium.

This experiment showed that crossing lines of independently derived transformants could result in significant strain heterogeneity and that the resulting pools of mated strains were likely highly diverse.

Mated strain 7124.2.557 (Cross E) was created by mating a pool of transformed strains derived from Y-7124(7124.2.535-539) with a pool of transformed strains derived from CBS 6054(6054.2.356-359). Mated strain 7124.2.558 (Cross F) was created by mating a pool of strains 7124.2.546-549 with a pool of strains 6054.2.356-359.

### Example 7

### Shake Flask Fermentation of 7124.2.557 and 7124.2.558

Cultures were started by inoculating a swath of colonies into 100 ml propagation medium (2.3% (v/v) black strap molasses, 26.8% (v/v) filter-sterilized pre-fermented corn stover hydrolysate (EdeniQ), 2.4 g/L urea, pH 5.55) in a 300 ml flask and grown for 48 hours at 30° C. and 200 RPM. Triplicate flasks were inoculated to a starting OD₆₀₀ of 3.5 (≈0.53 g/l dry weight of cells). The fermentation was carried out under oxygen limiting conditions with 50 ml of medium in a 125 ml flask, agitation at 100 RPM, and at 30° C. Fermentation medium composition was: 53.6% v/v filtered pre-fermented corn stover hydrolysate (EdeniQ), 60 g 1⁻¹ xylose, and 2.4 g 1⁻¹ urea. Starting glucose concentration was 4.7 g/l, starting xylose concentration was 60 g/l, starting ethanol concentration was 0.85 g/l, starting acetic acid concentration was 0.27% w/v and pH was 5.1.

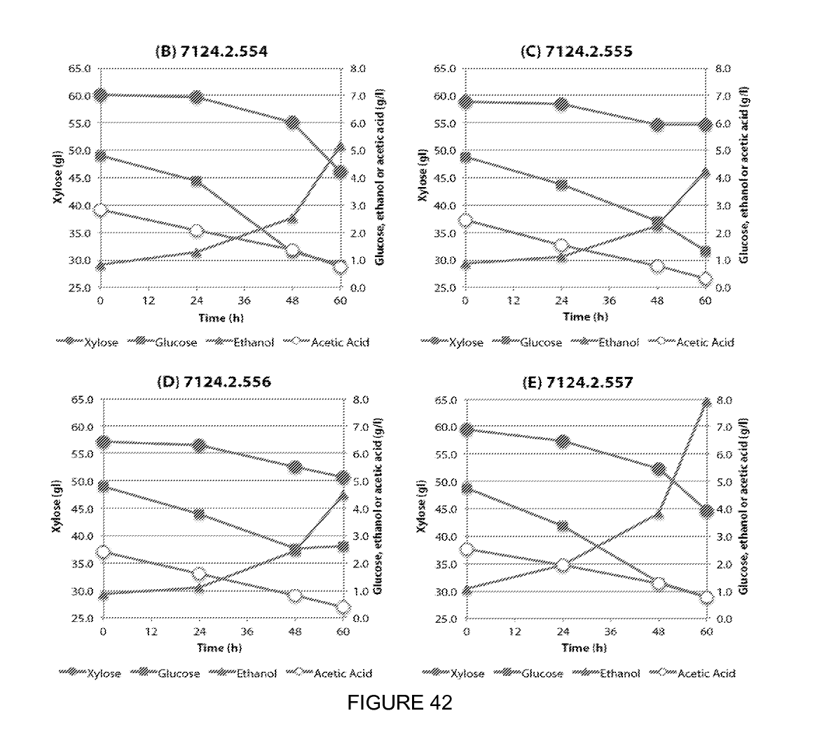

7124.2.557 was able to ferment glucose and xylose in the presence of acetic acid in medium containing industrial corn stover hydrolysate with a final ethanol yield of 6.87 g/l, compared to 4.6 g/l by the control strain CBS 6054 in 60 hours resulting in a 49.3% increase in final ethanol yield. 7124.2.557 consumed 18.66 g/l total sugars in 60 hours compared to 12.79 g/l total sugars by the control strain CBS 6054 resulting in a 45.9% increase in sugar utilization. 7124.2.557 had a specific yield of 0.368 g ethanol produced/g sugar used, compared to a yield of 0.359 g/g for the control strain, a 2.5% increase (FIG. 42).

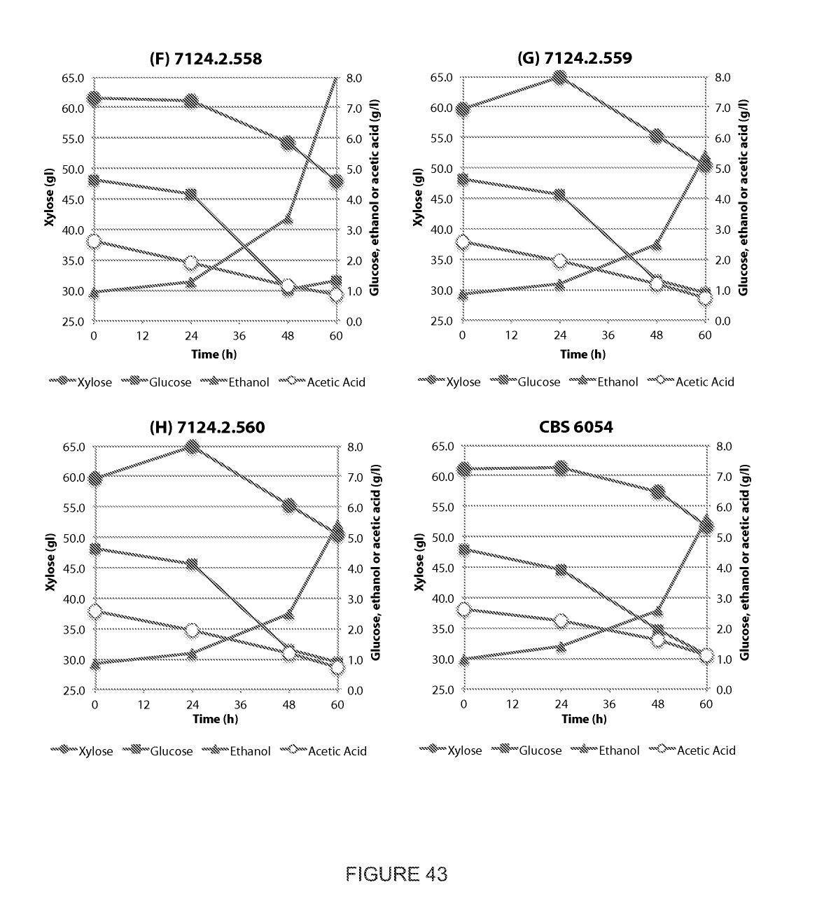

7124.2.558 was able to ferment glucose and xylose in the presence of acetic acid in medium containing industrial corn stover hydrolysate with a final ethanol yield of 7.09 g/l, compared to 4.6 g/l by the control strain CBS 6054 in 60 hours resulting in a 54.1% increase in final ethanol yield. 7124.2.558 consumed 16.89 g/l total sugars in 60 hours compared to 12.79 g/l total sugars by the control strain CBS 6054 resulting in a 32% increase in sugar utilization. 7124.2.558 had a specific yield of 0.419 g ethanol produced/g sugar used, compared to a yield of 0.359 g/g for the control strain, a 16.7% increase (FIG. 43).

Notably, the unadapted parental strain NRRL Y7124 was inoculated as a control but failed to grow in this medium.

This experiment showed that the various crosses all exhibited better acetic acid tolerance than the best of the parental strains and cells from two of the crosses showed significantly higher ethanol production.

### REFERENCES

1. Boles, E., and C. P. Hollenberg. 1997. The molecular genetics of hexose transport in yeasts. FEMS Microbiology Reviews 21:85-111.
2. Gietz, R. D., and R. A. Woods. 2002. Transformation of yeast by lithium acetate/single-stranded carrier DNA/polyethylene glycol method. Methods in Enzymology 350:87-96.
3. Jeffries, T. W., and Y. S. Jin. 2000. Ethanol and thermotolerance in the bioconversion of xylose by yeasts, p. 221-268. Advances in Applied Microbiology, Vol 47, vol. 47.
4. Jin, Y. S., H. Y. Ni, J. M. Laplaza, and T. W. Jeffries. 2003. Optimal growth and ethanol production from xylose by recombinant Saccharomyces cerevisiae require moderate D-xylulokinase activity. Applied and Environmental Microbiology 69:495-503.
5. Katahira, S., M. Ito, H. Takema, Y. Fujita, T. Tamino, T. Tanaka, H. Fukuda, and A. Kondo. 2008. Improvement of ethanol productivity during xylose and glucose co-fermentation by xylose-assimilating S. cerevisiae via expression of glucose transporter Sut1. Enzyme and Microbial Technology 43:115-119.
6. Lagunas, R. 1993. Sugar transport in Saccharomyces cerevisiae FEMS Microbiology Reviews 104:229-242.
7. Laplaza, J. M., B. R. Torres, Y. S. Jin, and T. W. Jeffries. 2006. Sh ble and Cre adapted for functional genomics and metabolic engineering of Pichia stipitis. Enzyme and Microbial Technology 38:741-747.
8. Lu, C., and T. Jeffries. 2007. Shuffling of promoters for multiple genes to optimize xylose fermentation in an engineered Saccharomyces cerevisiae strain. Appl Environ Microbiol 73:6072-7.
9. Spencer-Martins, I. 1994. Transport of sugars in yeasts—Implications in the fermentation of lignocellulosic materials. Bioresource Technology 50:51-57.
10. Verduyn, C., E. Postma, W. A. Scheffers, and J. P. Van Dijken. 1992. Effect of benzoic acid on metabolic fluxes in yeasts: a continuous-culture study on the regulation of respiration and alcoholic fermentation. Yeast 8:501-17.
11. Weierstall, T., C. P. Hollenberg, and E. Boles. 1999. Cloning and characterization of three genes (SUT1-3) encoding glucose transporters of the yeast Pichia stipitis. Molecular Microbiology 31:871-883.

It is understood that the examples and embodiments described herein are for illustrative purposes only and that various modifications or changes in light thereof will be suggested to persons skilled in the art and are to be included within the spirit and purview of this application and scope of the appended claims. All publications, sequences of GenBank Accession numbers, patents, and patent applications cited herein are hereby incorporated by reference in their entirety for all purposes.

What is claimed is:

1. An isolated Pichia stipitis cell, recombinantly expressing:
a. a xylose transporter;
b. one or more of a xylose reductase, a xylitol dehydrogenase, and/or a xylulokinase.

2. The P. stipitis cell of claim 1, further recombinantly expressing a transketolase and/or a transaldolase.

3. The P. stipitis cell of claim 1 or 2, wherein the cell further recombinantly expresses a cellobiose transporter.

4. The P. stipitis cell of claim 3, wherein the cell further recombinantly expresses a betaglucosidase and/or an endo-1,4-beta-glucanase.

5. The P. stipitis cell of claim 1 or 2, wherein the cell further recombinantly expresses an alcohol dehydrogenase.

6. The P. stipitis cell of claim 1, wherein the xylose transporter is selected from the group consisting of Sut1, Sut2, Sut3, Sut4, Xut1 and Xut3.

7. The P. stipitis cell of claim 1, wherein the transporter is substantially identical to any one of SEQ ID NOs: 46-51.

8. The P. stipitis cell of claim 1, wherein the yeast recombinantly expresses a xylose reductase, a xylitol dehydrogenase, and a xylulokinase.

9. The P. stipitis cell of claim 1, wherein the xylose reductase is substantially identical to SEQ ID NO:52; the xylitol dehydrogenase is substantially identical to SEQ ID NO:53; and the xylulokinase is substantially identical to SEQ ID NO:54.

10. The P. stipitis cell of claim 2, wherein the transketolase is substantially identical to GenBank EAZ62979 (Tkl2; DHAS; SEQ ID NO: 92) or GenBank ABN64656 (Tkt1; SEQ ID NO: 93).

11. The P. stipitis cell of claim 2, wherein the transaldolase is substantially identical to GenBank ABN68690 (PsTal1p; SEQ ID NO: 94).

12. An isolated yeast cell comprising a first and second expression cassette, wherein the first and second expression cassette each encodes the same xylose transporter, wherein
the first expression cassette comprises a promoter operably linked to a polynucleotide encoding the xylose transporter; and
the second expression cassette comprises a promoter operably linked to a polynucleotide encoding the xylose transporter.

13. The isolated yeast cell of claim 12, wherein the xylose transporter is SUT4.

14. A method of converting xylose to ethanol, the method comprising, contacting a mixture comprising xylose with the yeast of claim 1 under conditions in which the yeast converts the xylose to ethanol.

15. The method of claim 14, wherein the mixture further comprises a C6 sugar and the yeast converts the C6 sugar to ethanol.

16. The method of claim 14, wherein the mixture comprises at least 0.115% acetic acid.

17. An isolated yeast cell, recombinantly expressing:
a. a cellobiose transporter; and
b. a betaglucosidase.

18. The isolated yeast of claim 17, wherein the cellobiose transporter is substantially identical to any of SEQ ID NOs: 38, 39, 40, 41, 42, 43, or 44.

19. The isolated yeast of claim 17, wherein the betaglucosidase is substantially identical to any of SEQ ID NOs: 26, 27, 28, 29, 30, 31, or 32.

20. The isolated yeast of claim 17, further recombinantly expressing:
c. an endo-1,4-beta-glucanase.

21. The isolated yeast of claim 20, wherein the endo-1,4-beta-glucanase is substantially identical to any of SEQ ID NOs: 33, 34, or 35.

22. A method of converting cellobiose to ethanol, the method comprising, contacting a mixture comprising cellobiose with the yeast of claim 17 under conditions in which the yeast converts the cellobiose to ethanol.


SEQUENCE LISTING (94 sequences)

SEQ ID NO: 1 - DNA, 689 bp, Scheffersomyces stipitis
  promoter (1)...(689): PICST_37097 promoter
gacttaactattttcctttcggcagtttatacgagatcctcttcttttatctttcgttcaatggtattttcatttttttcgaacatgtaagtcgtagacgataaacgataaacaacagtgttgtggatcagttctcaattctgaacaatatgcgccaccaaaacggttaccttggtttctgcgagcaacttgtggtacgaagctaattaatttgcaagtgagcgtgatcatggaacatatttacaatggactgtaggatgtaaaactgatgatacagagtgggctgtggcaagctgtactatcatagaattcgatctttttatagcctggaatacgagatcatctggaacacaattgggttgggcccctgacttcaatacaggcttcgaacgagtttcagtaaatttcagatggaacaatattttattacttattatagtgaaatataacaagccccctcaatggatccataaacatatacatactgtatgtatgacattcccccttttcgtgggcagcgtttcagtatggaagagtgtcttactggcagaaatgcgatgggggtaaagttgacacgcttgcataattgtcgacgcttcccaagaggtataaatatggggagtttcgtctcttaacttgagctcctgttttctgttcttcattttttaaacaaaagtaaaatcaataca

SEQ ID NO: 2 - DNA, 705 bp, Scheffersomyces stipitis
  promoter (1)...(705): PICST_84653 promoter
ctaccgatctgaaacctgtgttgggctcgttgcacgggtgcgttcctacatgggcaaactctgaaaccagcaacaggaatgcctagaccaggtaccggttccataaaaagcaggtggaggctggttgcgattttgtggatgttttacatctggaatctgaaacacatctgcgggaactaaaacaattgtatataagtttcgctagatccaaagtagtttcatggctgaatctaaccaagcttgctgcacttttgtttagcaactccaaacttccacaaccctgttcagactcttattattgtagttgactgttgttgaatccaaatcaaccgcactcactttgttttccatatttttcaccgtttttcccacacattaaaagtcgtctgggacctggccaacaaatatttagctgttgcttgacaaagctattctagccagtctacagtcccaattgagcttcctttcaccataaacttcaccacaggccaaacagaaattgcattcatcaccagtgtgccaattgctccgctgttacggccaagaaagcttgagttacactaccgtctaaaaacaaagggttcgcacaatcgccattcgccgaattatccaatacccacattttcggtgaaaccgtatcgcagtcgtcagcacgctagagtggatagcttatagtctataaactgtgactcgtctctgtgtcca

SEQ ID NO: 3 - DNA, 1000 bp, Scheffersomyces stipitis
  promoter (1)...(1000): ACB2 promoter
actcaccatcgtgctctaccaatatgggcatgataggtccatttcattagcttacatctagacatggagttgataggaaaaaaggaaaaagacaaaaacatcaataacacaacatctccaagaaggttcacagaactaaaggtgtagttgtagtgtagttgtaggtgtagatactttttatttgttgattattgtcgactattcaccagtgaaaagttgatctcagtatagaataagttagagtgagaaaaccacagtagaggagatatcaagccattgacttattctacaaagactgtatattctacacaactgtcgcaattctagttcatggctgtctaggataattcaccctcgacgctgttcttaactgtctgctctattctttgctggtagacccgagctcaacattcccaacagaacctgacactctcaaattatggaggactgtatatacaatcccaagctttagaatatatatagcgattctcctcggatgaaccttagctcaaatctaaagagagaagtaaacaggaaagttgcatatcatacagacggtcccttctggaagctattagttacatatagcttcaggtttccctctcgccacatgaaagaaattccatgacactcttctgctaagatatccagtctctatccttgggctgatcctgcataacatccttcatatagcaaatatcacgtgcggatccccaccatttcagagtagccaaaacaacagtctactgagattcaacctctgttccacccagaggactctccacttcaccccccttcttcttcctaccggtgtactctagacattctatataacgagtggtacttgtttgaggtagtcgataagtcaattggtgtttctgacttgtgtaactcagctcgtctacttacacgagccatcaagtcagatcaatagtccaactacacaatagcaattatattcactatctaagtacgcccataaaaccacttatatacaacg

SEQ ID NO: 4 - DNA, 651 bp, Saccharomyces cerevisiae
  promoter (1)...(651): ALD1 promoter
gggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatataca

SEQ ID NO: 5 - DNA, 1000 bp, Scheffersomyces stipitis
  promoter (1)...(1000): BGL5 promoter
atctaatggactgcgtttcgttggcctgggagttgaagtaaatcggtcgtattaaccaagaattaatctaatagtgtttgaaaatgggcctccccgtccccaccgtggccacccctccttgtagtataatctttttattttttcagaacaaacatacaatttgccatggcgagttttagacgcgcagagtaaaatgcaaactgcaaagcataataatagccctgggtgttcagacatttttcgcccagttcgcacatttcagatgaagtcctgtattttgtttcaggtttttgtcccgcttttctctaccactaccgtattcggcagcagctccagaatacaccatattgaacaatcactccatcggtccaaacgtcagctagcttgtcacgcttaggaaccggacattctgttctggctctggtcacgagacgagcatgcccaggttttttgtctaaacctggtgatgcttcgtgcggagacatctccacattcggcattcgttccgcatgtatgcgcgtggggaaggataccgaattgggaactgttcccccgcgtattctgaatttttcatctcaagaacttcttgctcgtaggaaagccaactcagaaactggattacgagcgatttcagatgaagaaagtcgtgagtagccaaacttgtgtagctgaagggcattccccatcaactcagtccatccagcaaactcaagtctcgacaatatgagatccagtttaaagaatcttgtgttacattaaaaccaacttgtagatcacaaaatgcaatacacaatgttccacatcaaagccgatccaaacgtctcgcaacaaaaatcgcaaatccaacattataccgagacgtcccctgattacgtttctgtgcgcagtgtaaatatatatatagagtctcacatcccgttttgacgctactactcagtttcatatattgttcttatacatcttttagttctacttataacaactaaatatcattactatc

SEQ ID NO: 6 - DNA, 1000 bp, Scheffersomyces stipitis
  promoter (1)...(1000): CLG1 promoter
tgtgatgtggaattgaggaattgagtaaaacagaacagaatttttcaagtgctggattactggatctctccctacttatatacccggcactagcccacctgtgcttagtgggggtgaaccaaccgaaattcgattagtgtggggactgtgtgcaggcgcgggacaaagtcgacctttgggagcacactgggcctggtgtggccggattgggcaacttggttccaggcgcggtggacggtgagtggactttgactaatgagatgtaatgaggttttgtaatgggagtggaaaggatgacgatcttgatcatctagaatgtctctagagttggatgtctgacatataagatggttagctccgtgttcgtgttggttctgtcatgttccaactgtttgtatgtattatgacttcaccgtgttctggaaactttcgcggtataatttttcattccatttttcagatttttcataatgaattttctccatgaattttccattctcactacccaccaattcacactgttcatcagcacactcgattgcgagatttcccggtaaacgtcatgttcatctaattggctgtaatcaggaacaacctgctctgctcaacggcactatttcttttcttattattccgtgatctcgcccatctccatcttgtattgcacctacagctactactcatttctatttctttacctctctccatacttttgcatctcgaaattgcttttcttctctactattatcagctctccattaccttctctactgttttcttcctgtcgcgactttcttttgtaaccgcctctctgcctctccacaaacttccatccctgccgacagtactccggaatttcgtgccaccaaatcttttttgggctcccacggagtgagtgcttccgcctacacaaactgggctaatattagccaggtggcccgctgccatcgccgattgtttccaatggcttattagcctatctagcgagcgggacatgtct

SEQ ID NO: 7 - DNA, 1000 bp, Scheffersomyces stipitis
  promoter (1)...(1000): EGC2 promoter
agattttgtccatctgctgttctcctgcagacactgcgatggaagctcaatccataattttgtctgcaatatttacgagccagggctcttagaaatatttgcgagttaggtgaggtgattcgctgcggagtacataatataccggtgtacatggtgtactggtggaaagaagactgacagaatagtctcatgtcactgtagcacagcgtgagagcctcagacaatttttccaatatcgtgtcgggcatctcacaagtcagccgatattctagagccgcctgtagactcaaagttttgcgtcagggttctttggctcgaaacgtcctagacttaccagaccaggtaagccggaatggatctggtttcatcgccatatcagcgtcagtgtcatagaagtggcttaaatcgaaggaggggtgatcatgtgactgatcaattacctcattgagagggtatcccgcacttatgccccatttcccgcatggacctgaactcccaattttgtgtctgtctggggatagagttgcattaggcagtcaatgcagtttggctctggcggtgtcggcaatttcgaggtctgggccagtcccaccaaattcacattcttgtaacttccaaagcttttgagtcggcgtaaggtggatccagattttcagacaccagccatactgttaccgcccaggaatcagctgtctgcgattgtcgggaaccagctaccactggtgcaacagacatttacccaaactaagaaatcaggctgcgaaatctggggctctttggttccagatgtcggttcctgggctcggcacagttttctctacaatactcgggaagtttggccaccatcgttttttagattgtccccagatttgatccaggctatataagactctcggactgctctctgattccgatctaacgttcatcttgttcaaaatagaaaacagacacttatagattttacatctcttttgtcaaagtaattctaataccctataca

SEQ ID NO: 8 - DNA, 1000 bp, Scheffersomyces stipitis
  promoter (1)...(1000): ENO1 promoter
cgggcaatgggaacttgttgcctcggcgcacgtgacaaatgtgattatgtcgaaaactccgagtccgagagcagacagacggagtgcggtgggaaggaacaatcagatcagggagaccagcgagaatcgtacgctcctctagaactgtgttattgagtcattggagtggcttcaatggatatgtgccattgtggcggttatgaacgttcagaatgcgattgaggggtagagtgaatcatttttgacggtgccagccagagaggaacgcatggtgcaagtgaaaaatttcagctactgtttgagagcaattagaatactgttatgagccaataaactagtcaaaccaaatagccaatcaaccagccaacaagccggtggtagccagccttgggacaggcattggcacacaaataacctggtgtactgtaactacatcaccagccaccgtatcactgctccattatcagtgccatctcatgagcattggagctgttgatgcaagctgtcgctaatatgccgcaacaaattggactcatttttagggcaattctatccagtaccaataaagcacgaatcgctttatgaatcatagcctggccgtagcatttcagcaatttcgcaggttatggtttaacagcgacgtacaaaacttttcacagtcatatacggtatacccaaacatggattcgtggacttcggctcctccgttgaactcatattcgtaatccccattcagattgccctctcatgatgcccaccagttgcaatctggtgatcgcattatgcacactcttcgggtatcgggactgagtggtccagtttcgcacaaaattcgcacacggtgaacaagatggcccacacttttttcactcgacatataaagggaacgagatttcctccttgatttctcctggcattgcgtactgtgtattttttgcatctagtcaattatctgatttccagctaattacttgcttctttatcgattcccgcactaaca

SEQ ID NO: 9 - DNA, 1123 bp, Scheffersomyces stipitis
  promoter (1)...(1123): FAS2 promoter
tgttgatcatttgtaatagtcctagtatgataccaaaagtgaccgtgggtctatcatgatagggtggagatgatctttgatattccaaagcaaaagtgttcccttaaaccagtttagactgaaacaaccgaatgtaatcagggtagatgagaaggcattaagctgtggtgtttggctcaaaaagagattctacacaatattggactttgatttgtatattggctatacaagaatatggcaggccatactgatgctgaaaagaggttgttgaaaaaagttatgaatatagaactgaaaaatttgaactaattgggaaatgtccgggtaagacatggagactgcatagctggagagggccaaagtataccgggctcaagagcaccagccaagggggagtgtcggacagccgatgggtctgctaatgggaagggattggaagcgagttaagacggaaaaagaaaacgttttgttgaaaccactttggaccaaacatgagaattccagagctgtgtcaaatggaagctccagagttgggtgcaaaatctgagtataatatttgttgcgaaatccaaaatgcagaccttaactatgggctgagcatcttatctatctacgtatactcttatatatcggcactatagcaaaactttactggctgacacatctcggctgtaacataaatatctgttaaatccgcctcaacaaagtgttacccaatctcgtgctggccacctaaatttgagcttttaattgtgtgcttttaactgtgtggtcttaactgtgtgcccgtttctcagcctagccaacatttctcccaaaaattcgtgtgtcaaaagcgtgcaccgccaaattcctcaacaaaagcgcgtgaatgttgggatgggtctggcgctattctggcaaccgcacccgtgccgcagtacacaacagcttggctgcaacggtgtcgaaaattgttggaacctgctgaatcttttttcgggccgcatgcaggctgcagcccaccagatatcaatgctccatatataagtcgatgatttctacaaatgaacgaattgtatctcttttcttgaactgtagttctgatttctcacttctatagtaattctaatctcctttcacc

SEQ ID NO: 10 - DNA, 1000 bp, Scheffersomyces stipitis
  promoter (1)...(1000): HXT2.4 promoter
actaatttttgccgattcgccgaatagatgcaaaaagaccgctgggtcacaccgtaccagacagacaatgaacggaatctgttaccgccgagtggaaaagaaagcccgagtgtcagccttaggatctcgtgtctggaaggaaacagaaattgtcgctgtttgtgtgagtctgaaggtgtctccgctagacaatagtgcggactccaaaaaaataccgaatctactcaactccagattctgtgtagtgactggtagtttcacaaaatttcttttattggttcgaccctttgggaaaaaatactgcagttttgcctgacattttttggtttcttgtgtttgcatctaaatctaaatgagggacgtgaacggacaagtgcggagatgccccactattgcgaccttccaatagtacctgagcaggtctgtgtcgatctgtttctggacgtacaagaaagggttgagtgggtatctcactagcattctagcagaagcggccgtttgttggccggcccattgtttcctgttcaacgtcacgactcctgttggtgaagacattcaactacgaaaatgtcagatttgtgtttgtattcacaccattattaacttttgtctgacggaaacacttggataaactgcaatgtcctctaaaaaatactccagaattcagatcgtaaaactagtgggattatgcgtctgggtcgatattttttagggactggtgcatagtgtgggtgggagaaaaattataaaaattttaaataattttctttcataatatgcgggacccatactaaaagaaatgccttactatcaacagaatggttgctgctggtcaaatttggagccaatcctaattccaaagttttgtataaatacactggatccccctgttgatgttctcgagatttaattattcattatctcaacaatcacttgttagtactacagcctacaagcttctatcgctggtcgatctataaagtgcattaaagtagtaaaa

SEQ ID NO: 11 - DNA, 1000 bp, Scheffersomyces stipitis
  promoter (1)...(1000): LPD1 promoter
tgatccatgctggcgggcgacaaattcaaaagttgcagttgctgcagacgtgacttaaacggttcacctccgcagaaacgaatgcagcacggcgagagctggtaatgatatggatgaatatggacttcaatacagctacaatataattttagccacagtcgcatgtagaccttcgcagtcaagtccgtattgtacatcttggcaactgtgagtagcggtgtttgaagctggtctttgaaacagtgacaatccctacagctgcaaaatgagatagcggcattaatttgtggtcggtagtaaaaccgcgaaattcgtcagcatctgaccccgtaaacgtccgggatatgaaggtttgtaagctggcatagttaagctggaatcaaagcttagaaatgaagaatggacaaaagagtgacgcgggatggacagatgacactgcggagatagtagtatatatgactaacttgtgtttcgaccactgtttaagctggaatgtcagtaattcgtatcatacgattcattagtataaaagtatatgctcacacctatttgcaactgtcagaatatttttgtgcaagatttgctaatactggaagttatttctctgaatatacttaatacttcagtatttccctaaccgaaaaaacatattcaatatactagtactatagaactaaccctatacatcagtactaccgccatttgatcccgattatagttctatagccgtatagtactattcgtatagatctcagttcattagcactgagaccagccgctgcactcctacagctctatttttccgacacgcccggcggcagagccaataaccttcgcgctcctgcagataacgcaactcaccacggacatcagtgaagcatttttgtaaactaccagtggaaaatccatcgaatctgaaggcatctttccaattcttgtaagctgctgctgaaaagtaattgaacactagcagaaagtccgttttcttcctatacgaaaa

SEQ ID NO: 12 - DNA, 1000 bp, Scheffersomyces stipitis
  promoter (1)...(1000): LSC1 promoter
gaacatcttaatatttaagttgtgattcgactcggattcccatttatggagtgcttgatttgtcaacggagtggcgagttatcttttcattggctgtagttgtatttgaagttcggatcagttcccgttgttacggttacctgactatatgacaatgtctagtttctgtctggctcctgccgacaaataccgacgagatgcccaaccgtggtgtctggtcgcggcacattggcgacacttcgatccaggttatctttttcatcagagcaccatattccagcattggaggaccaatgcttttcgggctcaacccggatgtgatgtagagaatgtagatattttgacgagtagatttgagctcaaaatgggtcgaaacaggcgagatcgaagtggaatggtggaaccatcttgtctcatcgcctaactttggtgggcttgtttctcccactaattctcggcaacagtgccagggtggctctctacagtcggtatgtctcgctattttgcaaagtttcagaaatttctcgctgtttcatgcatacatgcaaagtgggacgtgtattctctcagtaagtctcagtaagtctcagtaattcctcgtggaggtgtcgctattttcgcgcagtccaacgactattgctcagcgacctctccctattgttacccgtgtcgtgccgtggttcccctcacacccccggcggattcgtcacacccgaaaaccaccagcaaaaacaaaagccgatccaccaacaaaattcaggtcgtgaaaaaatcacaatcgagacttcgcttttcttctctccagtttgctcttcgagtctcctgatattttcccgattgtcgtctagatctaccagtcagcttctgtcctttaacggttcactgccatccaatttagtctacttttatctattagtttggaagtaaccaaaaaagtatctcactactgtttgtctctactttctcacgtactctaccactgattccaccagaactcacc

SEQ ID NO: 13 - DNA, 1000 bp, Scheffersomyces stipitis
  promoter (1)...(1000): MEP2 promoter
ataacactgggaaggttgagagaataaacgaatctgtcgaaattcaagagtattgtttgaaactaaaaaatgccgttgctagcgtcagcgactaagcccgatctgatgagtcaagagtcaaaatcttaattaataagctcgatattggatattgattttaatgctatttcctcccaatgaacaatcatttgttgattacacacttttctcattggatatcacagcgcgactttgaccttgcatgtgctcggtccccaagtaccccataaggaaatacgctttcttttgtttacgtcggccaaacgtcattggtcgtttcagttcagatacccaaatctgtttgctggtcgcccggttccggttgatgagtatagacaggaactacttcttaatggtaatgctgattgccttcaaccgtaatcgctggctctgccgatacctgcacacagcttatcagtaaattggtagtaattgggtcggtgcggcttgggttcaacagtatgcagtgactagattcgaccgttgctgatggagcgatttcattggccgatgataaacgattataccggctgctaacaaaacacgacaagaaacttcacatttgtggaattgcagttgcaggcaatttaggcatgtacccaatgaaaaataatgcaagcagagcaaataaggcaatatctcctgccatatgaaatatttgagcctaccctacaaatataaataccaagcaagtcctattattacctacaattgcaagaggagattttcttctgtttctttctttgtctttgttctctagcaaatcctttcgtaactaacgttgtttttctgacgtacacatcagatctcgactacaggtctcataataacatagatctcacttgtatccagatcggtctcaagttgtcgatcgagctacaacccaattgcactatacgcatagattctcactacagtagccaaacaaaggttagtcaaaagatacccttatatacacaaa

SEQ ID NO: 14 - DNA, 1000 bp, Scheffersomyces stipitis
  promoter (1)...(1000): PGI1 promoter
gccgttgatagtgcttgatcagtgtgcaggacttcaatgagatccagcctttctgttagactggatcagctaaggaggtaaatgagccaataaaaggctcattgaagtattgaaataagtaaagaaacgtaaaaagacctttgattctgaagcagtgactgttattattttcctcattttgttcaaaccgacttacagataaagaacaatcagagggaaacggaaaattgaagaatatcacgtgatcaatttctcctttttccaccaccttctctatggtgtcctcctggtatactggagaaagagtactgagtactggagtattggtctgaacgcaaccgcaagagttccggccgtgttcacgagacccggaaggtgcacgctgtttgttcattagaagagatgccgactctattagaaggtgtctgcaggatattcactcctcaatgaagctatgaaccatcacgaaagagaagaactggcaaggacagaaaggataatgactaaatgttagtaaagtagagatccgagctgaagcgagtacttccagtcaattctgtctaattcacttcattgaatctcaggtctcggctgctatggaacgtcaaaggcattagtaacagtccggagttcgcttacaaagacattccccagttttctcgtcggcaaacctcccgccatatttcccacttgggcccagtgtggaagtcaccactccatcctgtgtgggtgttaattacctgattccaaggcatcacctggtggaaggactgtccttgaacagggcatttgcacccatgccgcagtatacaatccggacgcagacaggctggaatagattcggccgatttgcgaaaaataaagtatgggagttccgagcattttccactatatagtgaggccaatataaagagttgaggattccttggaggcattggtttttttcgagtttgtattgaaccttacaaccattgctacacgtatacgttacaattgcaca

SEQ ID NO: 15 - DNA, 1000 bp, Scheffersomyces stipitis
  promoter (1)...(1000): TAL1 promoter
agacatgtcccgctcgctagataggctaataagccattggaaacaatcggcgatggcagcgggccacctggctaatattagcccagtttgtgtaggcggaagcactcactccgtgggagcccaaaaaagatttggtggcacgaaattccggagtactgtcggcagggatggaagtttgtggagaggcagagaggcggttacaaaagaaagtcgcgacaggaagaaaacagtagagaaggtaatggagagctgataatagtagagaagaaaagcaatttcgagatgcaaaagtatggagagaggtaaagaaatagaaatgagtagtagctgtaggtgcaatacaagatggagatgggcgagatcacggaataataagaaaagaaatagtgccgttgagcagagcaggttgttcctgattacagccaattagatgaacatgacgtttaccgggaaatctcgcaatcgagtgtgctgatgaacagtgtgaattggtgggtagtgagaatggaaaattcatggagaaaattcattatgaaaaatctgaaaaatggaatgaaaaattataccgcgaaagtttccagaacacggtgaagtcataatacatacaaacagttggaacatgacagaaccaacacgaacacggagctaaccatcttatatgtcagacatccaactctagagacattctagatgatcaagatcgtcatcctttccactcccattacaaaacctcattacatctcattagtcaaagtccactcaccgtccaccgcgcctggaaccaagttgcccaatccggccacaccaggcccagtgtgctcccaaaggtcgactttgtcccgcgcctgcacacagtccccacactaatcgaatttcggttggttcacccccactaagcacaggtgggctagtgccgggtatataagtagggagagatccagtaatccagcacttgaaaaattctgttctgttttactcaattcctcaattccacatcaca

SEQ ID NO: 16 - DNA, 1043 bp, Scheffersomyces stipitis
  promoter (1)...(1043): TDH3 promoter
agatgactctgtagaaagttgagtcaaatgctgattaatttggttctattatgcctctcgtagaagattgcaaaagagcaactggatgaggtgctatcaagtgatgcgaagagaacctgcaaacaggccagagtacatgccgtgggttgatctctggtcgagtgtgctggctacagccttaagtacggagagtacagctacagggtggtttttgctgggctacagcattgcagtttgaaggttagagtgtagaatgtagcagacggcttaaggctggtggagtttagtcgaaactcgttagtatttccgtgaaggcagccattgtgaaaattgaacatcacctgaggtattttagccaccagaagcggcggtacggaagaaagtgtgtacaatggttggtggtggaattgcgtgcatgcctgatggggcaatattaattagatagagctttggtgatattagtggataatagaattcacagagaagacatcaggagcaatttccaagagccattgatgatgtaattgccccaacagcaagattcagatctgacaattgaccaccgttttgtagaagcaaaaaatcgtagattatcaccaagagggtttttcaccgaaccagcaaatagaaactattccgtagaactcgcccaggcttttttgctagcactttccagcagtagaaccgtccaattaagtcaacaggaaccattgaggtcgagcccaaccacctgaaccccctcacggtcgtgtccctattattgatccagagggtgccagtttcggtagccaatattggttcatgggtttctatggcccggagtgagtttgcaggttggccccggcgccgtctgcaggatgggagttatagcggccaaacttcacatttcgaaatcctgctgcagccaatctgaagaattaatataaattcgtgtcgaatcgccgtctgtgaaatttcagtacttgattttcttttcttcttctttttctcttttgtttcttcagaatcaattcacattttttcttccctataaacaattcatc

SEQ ID NO: 17 - DNA, 1000 bp, Scheffersomyces stipitis
  promoter (1)...(1000): TDH3 promoter
ttctattatgcctctcgtagaagattgcaaaagagcaactggatgaggtgctatcaagtgatgcgaagagaacctgcaaacaggccagagtacatgccgtgggttgatctctggtcgagtgtgctggctacagccttaagtacggagagtacagctacagggtggtttttgctgggctacagcattgcagtttgaaggttagagtgtagaatgtagcagacggcttaaggctggtggagtttagtcgaaactcgttagtatttccgtgaaggcagccattgtgaaaattgaacatcacctgaggtattttagccaccagaagcggcggtacggaagaaagtgtgtacaatggttggtggtggaattgcgtgcatgcctgatggggcaatattaattagatagagctttggtgatattagtggataatagaattcacagagaagacatcaggagcaatttccaagagccattgatgatgtaattgccccaacagcaagattcagatctgacaattgaccaccgttttgtagaagcaaaaaatcgtagattatcaccaagagggtttttcaccgaaccagcaaatagaaactattccgtagaactcgcccaggcttttttgctagcactttccagcagtagaaccgtccaattaagtcaacaggaaccattgaggtcgagcccaaccacctgaaccccctcacggtcgtgtccctattattgatccagagggtgccagtttcggtagccaatattggttcatgggtttctatggcccggagtgagtttgcaggttggccccggcgccgtctgcaggatgggagttatagcggccaaacttcacatttcgaaatcctgctgcagccaatctgaagaattaatataaattcgtgtcgaatcgccgtctgtgaaatttcagtacttgattttcttttcttcttctttttctcttttgtttcttcagaatcaattcacattttttcttccctataaacaattcatc

SEQ ID NO: 18 - DNA, 1000 bp, Saccharomyces cerevisiae
  promoter (1)...(1000): TDH3 promoter
ctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttgaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 19 - DNA, 501 bp, Saccharomyces cerevisiae
  promoter (1)...(501): TDH3 promoter
aacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 20 - DNA, 500 bp, Saccharomyces cerevisiae
  promoter (1)...(500): TEF2 promoter
gggcgccataaccaaggtatctatagaccgccaatcagcaaactacctccgtacattcatgttgcacccacacatttatacacccagaccgcgacaaattacccataaggttgtttgtgacggcgtcgtacaagagaacgtgggaactttttaggctcaccaaaaaagaaagaaaaaatacgagttgctgacagaagcctcaagaaaaaaaaaattcttcttcgactatgctggaggcagagatgatcgagccggtagttaactatatatagctaaattggttccatcaccttcttttctggtgtcgctccttctagtgctatttctggcttttcctatttttttttttccatttttctttctctctttctaatatataaattctcttgcattttctatttttctctctatctattctacttgtttattcccttcaaggtttttttttaaggagtacttgtttttagaatatacggtcaacgaactataattaactaaac

SEQ ID NO: 21 - DNA, 1446 bp, Scheffersomyces stipitis
  promoter (1)...(1446): TKT1 promoter
tctgctacagtattaaactatgctactataacctactgctgtgtaacttttactgtgatttcatgatgttatagcagctgctaccattatgctgtacaccttagtgtgatatacttgctgttatggactagtgttcactgtactgttatgctactctatatatttgtgctactttactgctcaaatagttgatcatattatccaacggcaagaccttctgcgaaccgacgggattccaccatcatctcctagcgagtggttgttgtagttatatgcctcggtcggaagtcgtgggaacaacgagctccggtggtggatcgaataggcacacccaaaccggagtcactgccggcaaaattgtctaccttctagcgggcggaccctaagactccgagttggccaaattggtgcgagcgtggaaaattataccggacggtggtggggcgacgattgcaaaatagtgagcgaactagatatttggaatggacatagaagcagaaatattatcaaatagacataacgaaacgctaccagatgtataccaagtccgagatggaagtcagatcaaagtcgttatcaatagcctatgtaaatttgcgctttagtaagagacagcccctccccataatctccctgtaggagaatatgctgctacaggaaaccaacagtagctgcaagactccagaccttctgtgccaattccaccacgcctttagcacccgatccagcaaattgagcacattcgagggttgtatcatgtaaatgctccaagcccgagcaagcatctactagaagaccacacaattttattcgaggagaccggaattaaattagttgtaatggcgtggacggtgacgtagcagtgaagcagtgattctggaacttttgcctggtcgaatgtgccccgcggtgggtctagtttccattattaatgtaccactacatcacgatccgtcagggtataaggaaggtgaaaattagtaaggaaaccattgggccatggcgagatccgggtcgagggacgagcgaccggagcggcaccacctaccgttcggaagtgagcatagatgctaatgattcgcttacacagaagtaccagagttcatgctactcaaaccaactactccacttaagctatgattggtatgcacgtgagttgtatacttaatcaggtcggccccaccctcgcccccaggtcggtgaaaaattttagtgcgtgccaacatatttcattattactactgaatcgctgcagttgataaacccccacggttggaaattgtccactgctgcgtctgaaaaatatatataggaattggaatttccagcccacaacaaaatttggcagttcttcttttccttctcttctctctttctggtccagtggaattccttactattcctatcgcttttgtatcttcaattgccaccagacttccatttgcc

SEQ ID NO: 22 - DNA, 401 bp, Saccharomyces cerevisiae
  promoter (1)...(401): TPI1 promoter
agattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaa

SEQ ID NO: 23 - DNA, 1000 bp, Scheffersomyces stipitis
  promoter (1)...(1000): XUT1 promoter
tcagcataatgaacttcccgttgattctaccgccccctctccttattacgtgaataatgcaggtcgcggtacattttttatgcaaccccatcatatattcaccgacttccgagggcgcatctacattacagtagggaagaaaatccgaaagggcaatcccccagaaatattatttctcttgacttcacatactactttgtgcgtggtaaatgtatccagcaaaactaattaccctagaaaatattcacctaactaccccaccccacatcatttgcggaagtagaaaaagcttgctaggctgaagttgtacatgcaaataatattccggacaatagccttggtgtgtgtttgaatgtgaaaagaaaacccgaaccaatgtcggtgagaacactacttacgagttttggcatttgagttttggcatttgagttttggcatttgagctttggcatttaagttttgccgtttggctagtcataataggtagttttgatatcatgatgttccttttctactcgattgatacttcgatggatggattgctttcccgatgacaagcttccatggggctgaaaatacggcgctatgcattcccaaaaaatgcccgcaacaatattcctccggggtagaaaatcaccaccacttaaagtttagaaggtggatccttcgtccaattttcggatcaggagtgcataaaaatcacgagcaacctccgcatatttactccacgttacggaataaccttcctagacatcagtgcatttctgactttcgtcggaatgatttgactttcgacttgggacacaaaacctcacctacatgatgcatgaattattgaggtgaaattaatgtggagtatggggcaagaaggtgcttaccaatacggtgctgcaattctgtggggtcaataatcatataaaagaaatgaatctgctgatacatgaactaatttgaagtagtaatttaatcaaataattcacattcaactaatatattcaaa

SEQ ID NO: 24 - DNA, 1056 bp, Scheffersomyces stipitis
  promoter (1)...(1056): ZWF1 promoter
gtggggaggaacaggtccagcaccgtgcggcttgaacgctacgctagacctggtctagcgagagagccagtatatatatagagcaatggtggaagaatccgcctgcgccagagctggagatatatatttgagagcattgagttcaacgagaaattgtagtggtagttgtaggtgtagatgtactggcttagtgatcaggaagctacgggaagattgtaggtgcatcaccgtagtgcgaaattctgccgtgccagagaactctccgacgctggtgccaccgaagagtgataccgaagagttataccgaagagccagatctgaagtcttagtcaatggaatcattgtcgagtcattttggaccccccatggcatcatgtgcggactcgtacgtctgtatttggagtcaaccaaacccccgacagaatggtgttactatttgggtgcccccacggcagatgtagctccatccctgttagtaatacatgagttcgggtctacattctactaatttttcgcttcgggcctgacaaatttcacagcggactgtgactgacctgcctgggcctagaacagtaccacgaccacgaagagagctaaatccgatgatcatgtccccagaatttggtggctattcaaccggtggccaacacgagcataccagacggcccagtgatgttgagccagttgaagcctatgtattcggctgggtttgtccgatagttgtacccctattagagctcttgccttcgcagactgtccatgctaaattagcggtgtcgctatttttctgccattttttccgtaccgcaactcagcatttctcactaattgcgacagcacactctccccaatgctcggaaatcgcattcgcactcgcacccactcgcacggtgatttcccactatataagcgccggatttttctccatgcatgcgggcccgatttttcagcttctcctgacttttctctggttgtaatcctttctacttttgccccccccaaacagccaattgggatctaccttttcatttagaaccacctacatacccctacact

SEQ ID NO: 25 - protein, 337 aa, Zymomonas mobilis
  alcohol dehydrogenase (ADH1)
MKAAVITKDHTIEVKDTKLRPLKYGEALLEMEYCGVCHTDLHVKNGDFGDETGRITGHEGIGIVKQVGEGVTSLKVGDRASVAWFFKGCGHCEYCVSGNETLCRNVENAGYTVDGAMAEECIVVADYSVKVPDGLDPAVASSITCAGVTTYKAVKVSQIQPGQWLAIYGLGGLGNLALQYAKNVFNAKVIAIDVNDEQLAFAKELGADMVINPKNEDAAKIIQEKVGGAHATVVTAVAKSAFNSAVEAIRAGGRVVAVGLPPEKMDLSIPRLVLDGIEVLGSLVGTREDLKEAFQFAAEGKVKPKVTKRKVEEINQIFDEMEHGKFTGRMVVDFTHH

SEQ ID NO: 26 - protein, 738 aa, Scheffersomyces stipitis
  beta-glucosidase BGL1
MTAFDIEGILSQLTLEEKVGLLAGIDFWHTYAVDRLDIPSLRFSDGPNGVRGTKFFDAIPSACFPCGTALAATFDKQLLRDTGKLMGVEAKAKGAHVILGPTMNIQRGPLGGRGFESFSEDPHLSGHAAAAIVNGIQEEGIAATVKHFVCNDLEDERNSSNSILSMRALREIYLEPFRIAIKHANPKALMTGYNKVNGEHVSQSESIIKDILREEWKWEGTIMSDWYGTYTSDTAIRAGLDIEMPGPTKFRSLSEILHMVVSKELHIKHINDRVRNVLKLVQFAQGSGVPQNAPEGTSNNSAETSAKLRKIALDSIVLLKNTGILPLSKDSSIAVIGPNAKFAAYCGGGSASLASYYTTTPYSGIASKTTTPPKYSVGATGHRLLPDLASQVINPITGSVGVNAKFYSEPSTSERRNLLDEYNLIDTRVNLFDYISTSRARNEPFYIDFEGDFVPEETASYRFGLAVFGTADLYVDNKLVIDNSTNQKKDEHFVGSGTREEHGVIQLEKGKNYRIRVEFGSAHTYTFSDPNAEFHGGGSLKIGCIKVVEPEEEIRRAIEIAKTVDQVVLCIGLNLEWESEGYDRPDMELIGLQNKLVEEIIKANPNTIIVNQSGTPVEMPWLPKAKAVVQAWFGGTEGGNAIADVLFGDVNPSGKLSLSFPFKNFDNPAYLNFTTDNGRVLYGEDIFVGYRYYEKLNREVAYPFGFGLSYTSFKIGDLKVQVLDQDNIEISVNIKNTG

SEQ ID NO: 27 - protein, 851 aa, Scheffersomyces stipitis
  beta-glucosidase BGL2
MTPSVKQPVPKELDIEYLIEQLTIEEKVSLLAGKDFWHTQNIDRLNIPSVRVSDGPNGIRGTKFFNSVPSNCFPCGTGLAATFNKEVLLQAGELMGKEAKMKGAHVILGPTCNIVRSPLGGRAFESYSEDPVLSGHAAANVVKGIQNQNVVACLKHFVANDQEHERKAVDEIMTERALREIYLKPFHIAMRDAYPKALMTAYNKINGVHVSQNKKILQDLLRGEWGYTGTVMSDWHGVYSTKESLDAGLNLEMPGPTRFRQQVPTLHAIQTNEIHTDVIDDNARAILRLVNESLKAGIPDDVIESPNPTKEASDLLRKAGDESIVLLKNENNILPLSKTAVKGQEKIAVIGPNAKAAQDSGGGSASLNAAYKITPYEGIESKIIEGGNSVSLDYSLGAFLDRNLPDVGNTLINEEGKKGITAKFYKQAPGAADREHFETFTLSTSKIFLSDYKSKHLKPGQLLFYADFHGIYIPDETGDYEFGASCLGTAQLFVDDELVVDNKTKQVKGDAFFLGLGTREERGVKKLEKGKKYNIRVEFGSSPTFTLNKAALEGGGVFFGIRMISTAEAAIAKAVAVAKEADKVILVVGISKEWESEGFDRPTMDIPGATNELVDAITAVNKNVIVVNQSGSPVTLPWINKVQGFVQAWYGGNELGNTIADVLFGDYNPSGKLSMTFPKRLQDNPSYLNFASTHGQVLYGEDIYVGYRYYEKVGVEPLFPFGYGLSYTTFELKDLVVEYDQEIINAKVSVVNTGKVDGAEVVQLYVSQVNPSINRPVKELKDFGKVFVKAGETKTLELSVSVKEATSFWNGYKNKWQSEKGKYKISVGNSSDNITLEDEFETSKTYFWLGL

SEQ ID NO: 28 - protein, 738 aa, Scheffersomyces stipitis
  beta-glucosidase BGL3
MTAFDIEGILSQLTLEEKIGLLAGIDFWHTYAVDRLDIPSLRFSDGPNGVRGTKFFDAIPSACFPCGTALAATFDKQLLRDTGKLMGVEAKAKGAHVILGPTMNIQRGPLGGRGFESFSEDPHLSGHAAAAIVNGIQEEGIAATVKHFVCNDLEDERNSSNSILSMRALREIYLEPFRIAIKHANPKALMTGYNKVNGEHVSQSESIIKDILREEWKWEGTIMSDWYGTYTSDTAIRAGLDIEMPGPTKFRSLSEILHMVASKELHIKHINDRVRNVLKLVQFAQGSGVPQNAPEGTSNNSAETSAKLRKIALDSIVLLKNTGILPLSKDSSIAVIGPNAKFAAYCGGGSASLASYYTTTPYSGIASKTTTPPKYSVGATGHRLLPDLASQVINPSTGSVGVNAKFYSEPSTSERRNLLDEYNLIDTRVNLFDYISTSRARNEPFYIDFEGDFVPEETASYKFGLAVFGTADLYVDNKLVIDNSTNQKKDEHFVGSGTREEHGVIQLEKGKNYRIRVEFGSAHTYTFSDPNAEFHGGGSLKIGCIKVVEPEEEIRRAIEIAKTVDQVVLCIGLNLEWESEGYDRPDMELIGLQNKLVEEIIKANPNTVIVNQSGTPVEMPWLPKAKAVVQAWFGGTEGGNAIADVLFGDVNPSGKLSLSFPFKNIDNPAYLNFTTDNGRVLYGEDIFVGYRYYEKLNREVAYPFGFGLSYTSFKIGDLKVQGLDQDNIEISVNIKNTG

SEQ ID NO: 29 - protein, 814 aa, Scheffersomyces stipitis
  beta-glucosidase BGL4
MSIPEKVNLTTGTGWGSGPCIGNTGSVPRLGIPNLCLQHGPNGVRFTDFVTHFPSALAAGATFNKGLIYLRGKAIGREHKKKGVHIALGPVVGPIGLKAAGGRNWESFGADPYLQGVCGAATVEGIQDEGVVAVARHLVGNEQEHFRQVGEWDENGWEHLETSISSNIGDRAMHELYLWPFANAVRAGVGGVMCAYNQVNGTYSCENSYLLNNLLKEELGFQGFVVSDWGAQHTGVYSSLAGLDMTMPGEVFDDWLTGKSNWGPLLTRAVYNGTLSQERLNDMVMRILAPFFAADTITLPSENDVPNFSSWTFHTYGQEYMYQHYGPIVQQNWHVEARSNFSDNTALNTAREAIVLLKNPGHNLPIAKVDGVRRIFIAGIGAGVDPRGFNCKDQRCVDGVLTSGWGSSALNNPFVITPYEAIAKKARDQGMLVDFSNDVWELDHVEELADYSDMSIVVVGASSGEGYIEVDNNFGDRKNLSLWHNGDQLIESIAEKCKKTVVVVNSVGPVNLEKWIENDNVVAVIYVPPLGQFVGQAIAEVLFGEVNPSGKLPFTIARKKQHYVPIIDELGDDRSPQDNFDRDIYLDYRFFDKHNIKPRYEFGYGLSYSSFLVCDLKIKEIKAPLEYLPYPEEYLPIYKTCEDDICDPEDALFPHDEFDPVPGYIYPYLYNENVRTLEDDSHFDYPHGYHPEQNSVPPLSGGGLGGNPELWQTLYEVDAEVKNDGKYRGAYVLQLYLELPSTILPSPPRILRGFEKVFLEPGETARVSFKLLHRDLSVWDTYSQQWIIQTGTYKVYLSSSSRKVELSGEIDIGC

SEQ ID NO: 30 - protein, 843 aa, Scheffersomyces stipitis
  beta-glucosidase BGL5
MGVQELDVERLIEELTIPEKISLLAGKDFWHTVPIERLNIPSIRVSDGPNGIRGTKFFNSVPSNCFPCGTGLAATFNKDLWVEAGELMGKEAKMKGAHVILGPTSNIVRSPLGGRAFESYSEDPLLSGHAAANIIKGIQNENVVACLKHFVCNDQEDDRRGVDTLLTTRAFREIYLKPFHIALRDADPGALMTAYNKINGIHVSESKEILQGILRDEYKYEGATMSDWFGIYSTKTALEAGLNLEMPGPTRFRLPIQTLHEVQANRIHTKTIDDNVRYVLKLINRALKADIPHDVVESANEDPAASEILRKVGDESIVLLKNEGNILPLSKTSVAGQEKIAVIGPNAKAAQDSGGGSASLTARYKVTPWEGIKKKIEEGGNTVSLEYSLGAFLDKNLPDVADILENEKGEKGVTAKFFKNAPGTKDRQQFAEYLLPTSKLFLSDFTDPGLELGELLFYADFEGYFTPEETADYDFGASCLGTAQVFVDGKLVADNKTKQTKGDAFFLGLGTREERGTVHLEKGKKYHVKCEFGTSPTYTLEASQEIGGVFFGFRINSPAEIEITKAVELAKSVDKVVLVVGLSKEWESEGFDRPDMDIPGATNQLIEEVLKVNKNVVVVNQSGSPVTMPWVDQVPALVHAWYGGNELGNTIADVLFGDVNPSGKLSMSFPKKLEDNPSYLNFGSINGQVWYGEDIFVGYRYYEKVKKDVLFPFGFGLSYTTFDFKDLSVAADDENVTVSVKVTNTGSVDGSETVQVYIEQSNPSIIRPVKELKDFGKVFLKAGETKSVEVKISIKEATSYWNGYQDKWQSEKDTYKVLVGNSSDNIILEGKFATSKTFYWLGL

SEQ ID NO: 31 - protein, 843 aa, Scheffersomyces stipitis
  beta-glucosidase BGL6
MGAQELDIEYLIKELTLPEKISLLAGKDFWHTFPIERLNIPSIRVSDGPNGIRGTKFFNSVPSNCFPCGTGLAATFNKDLWVEAGELMGKEAKMKGAHVILGPTSNIVRSPLGGRAFESYSEDPLLSGHAASNIIKGIQNENVVACLKHFVCNDQEDDRRGVDTLLTDRALREIYLKPFQIALRDSEPGALMTSYNKIRGIHVSESKELMQDILRDEYKYEGTTMSDWLGTNSTKAALDAGVNLEMPGPARFRTQLQVTHEIQSKRIHAQTIDDNVRGVLKLINRALKAGIPDDVVESANEDPASSELLRKVGDESIVLLKNEGNILPLSKTSVAGQEKIAVIGPNVKAAQDSGGGSASLTARYKVTPWEGIKKKIEEGGNTVSLEYSLGAFLDKNMPDVGDILENDKGEKGVTAKFYKTAPGTKDRQQFAERFLPTTKLCLFDFKDPELAPGEVLFYADFEGYFTPEETADYEFGASVMGTAQVFVDGKLVVDNKTKQTKGDAFFLAMGTREERGTVHLEKGKKYHVKCEFGTAPTYTLDPTQEIGGAFFGFRIDSPQETELTKAIELAKSVDKVILVVGLSKEWESEGFDRSDMDIPGATNQLIEEVLKVNKNVVIVNQSGSPVTMPWAEKVPALVHAWYGGNELGNTIADVLFGDVNPSGKLSMSFPKKLEDTPSYLNYGSINGQVWYGEDIFVGYRYYEKVKQDVLFPFGFGLSYTTFDFKDLSVAADDENVTVSVKVTNTGSVDGSETVQVYIEQSNPSVIRPVKELKEFGKVFLKAGETKSVEVKISIKEATSYWNGYFSKWESTKDTYKVLVGNSSDNIIVEGEFATSKTFYWLGL

SEQ ID NO: 32 - protein, 839 aa, Scheffersomyces stipitis
  beta-glucosidase BGL7
MTSRRFDIEEVLAELTLEERISLLAGLDFWHTVSVPRVGIPSLRFSDGPNGLRGTKFFDSVPSACFPCGTGLAATFDKELLFEAGQLMGEEAKHKGAHVILGPTMNMQRGPLGGRGFESFSEDPHLTGQAASSIIRGIQDKGIAATVKHFVCNDLEDQRNSSNSILTERALREIYLEPFRLAIKYANPICVMTSYNKVNGEHVSQSKRLLEEVLRQEWKWDGCIMSDWYGVYTANNAIENGLDLEMPGPPNFRKLTEIRSMVVTKELHIKHIDERVRGVLKLIKYALQSGIPENAPEDTLNNTPETRKLLRKLAHDSVVLLKNEDNLLPLSKDEKIVVIGPNAKYAAYCGGGSASLRAYYTTTPYDSIAAKTSTPIDYTVGAYGHRLLPGLAANLVNPITGKPGYNCKFYRETVGSPERTLIDEYNLDISYILLVDYYNDLAPDSVFFVDFEGEFTPDETAEYEFGASVQGTALIYVDNKLVVDNKTKQRRGNSFFNSGSAEEKGTLLLEKGKTYKVRIEFGSGPTFTCRQEGSTVVAGGGGINLGMAKVIDPEIEIHKAAKLAKEADKVVLNIGLNQEWEAEGFDRPDMELVGYQNKLIDAVLAANPNTVIVNQSGTPVEMPWLPKAKAVLQAWYGGNESGNGIADVLFGDVNPSGKLSLTFPFKTIDNPTYLNFKTERGRVLYNEDIFVGYRFYEKMGRDVAFPFGFGLSYTNFEFADVNVVVEELDDNLEVSVTVSNTGKVDGAEVVQIYIGKEDSDVIRPVKELKGFEKVFLKAGTQETVISTLSLKESVSFFDEYQEKWSVLAGEYQVYVGNSSDNANAIGTFVIERDFLWIGR

SEQ ID NO: 33 - protein, 483 aa, Scheffersomyces stipitis
  endo-1,4-beta-glucanase EGC1
MSTGFLTTKGTKIVDANGKQVVLVGTAIAGHLNMENFITGYPGHETEHKNVLKKKIGEEKFNFFFDKFYEYFWTEKDADFYKNELGFNCLRIPFNYRHFIDEEVDLFKIDPKGFERLDRVIDICSKYGIYTVLDLHATPGGQNQDWHVDSGIHKSSFFDFKVFQDSMVNLWIELAKHYKDNTWVAGFNPLNEPAVSQHKKLVNFYQRLHDEIRPIDPNHIFFLDANTYSMDFRQFPAPKDFIPNAVYSIHDYSTFGFPNIQGTLYTASDAEKEKLKRQYDRKVEYHHEHNVPVWNGEFGPVYASKERGDEDPDTINRARYQVLKDQLAIYKKGDPSGDGTPISWSIWLYKDIGYQGLTYVDPESKWYKVFGEFLLKKKKLGLDRWGNDIDPEYNQLYENLANHILENVPEKYHHALYPHHWTVLDWLFRVSKDQLFSQYAQYEYADLFVGLSFEELDELAASFKFENIKLRDELNDILKDYKN

SEQ ID NO: 34 - protein, 481 aa, Scheffersomyces stipitis
  endo-1,4-beta-glucanase EGC2
MSTGFLTTKNTKIVDANGTPVVLVGTAIGGHLNMENFITGYPGHETEHKKVLKKKIGEEKFNFFFDKFYEYFWTEKDAEFYKNELGFNCLRIPFNYRHFIDDEVDLFKINPKGFERLDRVIDICSKYGIYTILDLHATPGGQNQDWHADSGIHKSIFWDFKVFQDSMVNLWVELAKHYKDNTWVAGYNPLNEPASPDHSKLVNFYQRLQDEVRPIDPHHIFFLDGNTYSMDFRQFPAPKDFIPNSVYSIHDYSTFGFPNIQGTLYAGTAAEKDKLKRQYDRKVEYHLEHNVPVWNGEFGPVYASKERGDEDPDTINRARYQVLKDQLAIYKKGDPSGDGTPISWSIWLYKDIGYQGLTYVDPESKWYKVFGEFLLKKKKLGLDRWGNDIDPAYNKLYQDLIDHIHSNVPEKYHKALYPHGWTTQDYLFRVAKDMLFSQYAQHEYADLFVGLSFEELDELAASFKFENIKQRKELNEILKDY

SEQ ID NO: 35 - protein, 481 aa, Scheffersomyces stipitis
  endo-1,4-beta-glucanase EGC3
MSAGFLTTAGTKIVDAEGTPVVLKGAALGGHLNMENFITGYPGHETEHKLVLEKKIGKEKFDYFFEKFYEYFWTEKDAEFYRNKLGFNCLRIPFNYRHFIDDNGDLFKIKGKGFELLDRIVDICSQYGIYTILDLHTTPGGQNQGWHSDSAIHKSLFWDFKVFQDSIVNLWVELAKHYKDNVWVAGYNPLNEPAVSDSEKLVDFYKRLHDEVRPIDPNHIFFLDGNTYAMDFRKFPSPESYIPNTVYSIHDYSTYGFPNLEGALYTGSEEEKSKLKSQYNRKIEYQSEYKVPVWNGEFGPVYASKERGDKNPEVINRARFNVLKDQLEVYRKGDPSGDGSPISWSIWLYKDIGFQGLTYVSPKSKWYEVFGEWLLKKKKLGLDKWGNDIDPGYNQLYQNLVDHMEANVPEKYHKVLYPHTWTMEKYLARVSRDMLFSQYAQHEYADLFVGFSLEELDELAASFKFENLDQREELNQILKEY

SEQ ID NO: 36 - protein, 633 aa, Scheffersomyces stipitis
  glucose transporter HGT1
MVVIGRLIKGLAMGILSSLIPVYVAETIVKKASSISFVQLNAAISGLAMYYIAYFFPVLMPNEYSFRFAWAIEALPAIAIFILSFFLPESPKWLATKSRWGQAAKNLDKIKAYQNGKPQEKTNRDDREYVLRAYTSGPEIRNSSYDKIFGKKYWKHTVLGISTQVFVQLTSVQVLMNYFLFICELCGIEEHSLIFVSSALNVVQVIFTLVPLFILDNTRRRDSLTFGLIILSVSFLALFIIILTFGEHFTHEGFDLLFRFEMFDEPASAVLAIFLFINAVYSSTVLSASWLYAGELFPGPARAKGASICMCASWMVNTTMGLVLPILFKYIGPWTFATLALFSFVGGIALMFLPETRDLGEYELYSIFNFNNEPFPRQKLVSDKKKKKSKEAILGLESKEAVVNKPQFEHALTYEQQQGNGKVQLESLTGGFTTSPTSETVTEIETGVELETAREYMKPFSSETNSALRQETDPDSSQVEDILDIYTSGGALDEEDAISPNTYYSSDWSQGYQGVQGAATTDNYEEEEEEEAPAKISLISSKSSTRESTMKPPQSGNAYFHANREGSPIKAGLTYEPTTFLQFDSLRVALRTNILDRKKSEAKLRENANSTFPKGGVFISSISKSKMAAKTTP

SEQ ID NO: 37 - protein, 542 aa, Scheffersomyces stipitis
  glucose transporter HGT2
MSYEDKLVQPALKFRTFLDRLPNIYNVYIIASISCISGMMFGFDISSMSAFIGEDDYKNFFNNPGSDIQGFITSCMALGSFFGSIVSSFISEPFGRRASLLLCSFFWMVGAAVQSSSQNRAQLMIGRIIAGFGVGFGSSVAPVYGSELAPRKIRGFVGGIFQFCVTLGILIMFYICYGLHFINGVGSFRIAWGLQIVPGLVLFVGCFFIPESPRWLAKHGYWDEAEFIVAQIQAKGNREDPDVLIEISEIKDQILIEENLKSFGYVDLFTKKYIRRTLTAIFAQIWQQLTGMNVMMYYIVYIFNMAGYSNNANLVASSIQYVLNTAATVPALFLMDYIGRRRLLIGGAIMMMIFQFGVAGILGKYSVPVPGGLPGNPTVTIQIPEDNKSAARGVIACCYLFVVSFALSWGVGIWVYCSEVWGDSASRQRGAAVSTAANWILNFAIAMYTPSSFKNITWKTYIIYAVFCLVMAIHVYFGFPETKGKRLEEVGQMWDENVPAWRSSSWQPTVPLLSDADLAHKMDVSHKEEQSPDAESSSEEKP

SEQ ID NO: 38 - protein, 493 aa, Scheffersomyces stipitis
  cellobiose transporter HXT2.1
MLHIFVFLCTLSCTTNGYDGSMLNGLQALDSWQDAMGHPEGYKLGSLANGTIFGSVLSVSVAAWLSDKVGRRVAIIIGSGIAVVGAILQGASTNFAFFLVSRILLGFGVGIGAIASPALIAEISYPTFRPTCTTLYNTLWYLGAVIAAWVTFGTQHLKGSASWRVPSYIQAFLPAVQFVSLWWCPESPRWMIAKGREDEARQILFKYHTGGDQDDRAVRLVEFEIKEIKAALEMEKICSNSKYSDFLTIPSYRKRLFLLSFTAIIMQLSGNGLVSYYLSKVLTSIGIKSANEQLIINGCLMIYNMVIALSVAFVVYLFRRRTLFLTSISGMLFSYIIWTALSAVNQQRDFKDKSLGKGVLAMIFFYYLSYDIGANGLPFLYVTEILPYTHRAKGLNVMYGVQMTTLVYNGYVNPIAMDALDWKYYIVWCCFLAFELLIVYFFFVETYGYSLEEVAKVFGDDPNSSLIQSTSSNEKASIEHLEDTSSAEIGRVV

SEQ ID NO: 39 - protein, 540 aa, Scheffersomyces stipitis
  cellobiose transporter HXT2.2
MSKNQTIKDQIISISVSDGVEYDAQQEHEIDQYLYQKNSWWTYPHLRKLHLFVFLCTLATTTNGYDGSMLNGLQVLPAWQEAMGHPEGYKLGSLANGTLFGSVLCIFVGAWICDKIGRRNTITAGSGIAVVGAVLQGASTNFAFFLSSRILIGFGGGLCAIAAPALIAEISYPTFRPTCTAIYNTFWYFGAVIAAWVTFGTQNLNGGASWRIPSYLQAALPAVQFLTIWYFPESPRWMIAKGREEQARKFFFEYHTGGDQDERSVKLVEFEIKEIQAALEMEKICSNSKYTDFLTIPSYRKRLFLISFTACIMQLSGNGLVSYYLGKVLTSIGIESSNEQLIINGCLMIYNNVIALSVAFVVYLFRRRTLFLTSISGMLVSYIVWTALSAKNQQRNFEDKSLGRGVLAMIFLYYFFYDIGANGLPFLYVTEVLPYTHRAKGLNVMYGVQMVTSVYNGYVNPIAMDALDWKYYIVWCCFLTFELVIVYLFFVETYGYSLEEVAKVFGDDAHSPLISLDTGNGKTSIEHLEQISSVEVGKSV

SEQ ID NO: 40 - protein, 537 aa, Scheffersomyces stipitis
  cellobiose transporter HXT2.3
MSSLKQNQATVNQESTSDIEVQGDENKIESYLYLEGSWWKHKHFRFLNLCIWLIALTSTNNGYDSSMLNGLQSLPKWKLDMGSPVGPVLGALNNGNTFGVMLSFLLASWIADKWGRKKAIIGGSSLMVIGAILQGVSTNFGFFLFSRMVLGFGSGIAIVSSPSLISELAYPTHRAVATTLYNVFWYLGAIIAAWVTFGTRTLHSSYCWRVPSYLQGFLPLVQILFFWLVPESPRYLIANGRTEEARAILHKHHTGSSDDERAHALINFEVSEIEAALEQEKLYSNAKYSDFFTIPSFRMRLFLVVWTSVIMQLSGNGLVSYYLSKVLISIGITGVKEQLEINGGLNIYNLFVAGFIASNANKFKRRTLFITALSGMFITYVIWTVLSAINQQRDFSDKSLGKGVIAMIFLFYIFYNMGANGLPWLYMTEILPYSHRAKGVNIHNLVQTWIVIYNGFVNPIAMDAIQWKYYIVYCCIIVVELVVVYFTYPETSGYTLEEVARAFGDDETTHLRFINETSKDKFGVEHEESVDIASKTV

SEQ ID NO: 41 - protein, 547 aa, Scheffersomyces stipitis
  cellobiose transporter HXT2.4
MSDKLHNIKDQTDSLSITDHIDEQQNILNDPNTDINDLLFQTDGWWKYGHFRKLHFMIALIALASTNNGYDGSMLNGLQAIPDWQTTMGTPEGYKLGSLANGTMFGSIIAVSCASYLNDKWGRKFGVLFGSIISFIGGILQGASTNYAFFLVARIIIGFGVGIALTGAPAWIAELSFPSYRSSCTAVFNTLWYLGAILAAWITFGTEKLHGPKAWRIPSYLQAILPGIQVLTLWFCPESPRWLIDNGKEEKARSVLNAYHTGNVDDERAHALVEFEIKEIKSALELEKLYASSSYFDFLKIRSYRKRLFLVCFTAFIMQMSGNGLVSYYLVKVLRSIGYESPTEQLKINGCLQVFNIVISVGAALLTYRFKRRHQFLVCIAGMLLCYVIWTVLSAINQQRNFEDKGLGRGILAMIFLFYFSYDIGANGLPFLYATEVLPYSHRAKGLNLMYFTQLCTLVYNGYVNPIAMDAIEWKYYIVWCCVLAFELVIVFFFYVETFGYTLEEVAVVFGDDAGTTLHRLSSPVEKSAVEHLEDGNSSNEKIGERV

SEQ ID NO: 42 - protein, 534 aa, Scheffersomyces stipitis
  cellobiose transporter HXT2.5
MSQSKEKSNVITTVLSEELPVNYSEEISDYVYHDQHWWKYNHFRKLHWYIFVLTLTSTNNGYDGSMLNGLQSLSTWKDAMGNPEGYILGALANGTIFGGVLAVAFASWACDRFGRKLTTCFGSIVTVIGAILQGASTNYAFFFVSRMVIGFGFGLASVASPTLIAELSFPTYRPTCTALYNVFWYLGAVIAAWVTYGTRTIVSAYSWRIPSYLQGLLPLVQVCLVWWVPESPRFLVSKGKIEKAREFLIKFHTGNDTQEQATRLVEFELKEIEAALEMEKINSNSKYTDFITIKTFRKRIFLVAFTACMTQLSGNGLVSYYLSKVLISIGITGEKEQLQINGCLMIYNLVLSLAVAFTCYLFRRKALFIFSCSFMLLSYVIWTILSAINQQRNFEQKGLGQGVLAMIFIYYLAYNIGLNGLPYLYVTEILPYTHRAKGINLYSLVINITLIYNGFVNAIAMDAISWKYYIVYCCIIAVELVVVIFTYVETFGYTLEEVARVFEGTDSLAMDINLNGTVSNEKIDIVHSERGSSA

SEQ ID NO: 43 - protein, 534 aa, Scheffersomyces stipitis
  cellobiose transporter HXT2.6
MSQSKEKSNVITTVLSEELPVKYSEEISDYVYHDQHWWKYNHFRKLHWYIFVLTLTSTNNGYDGSMLNGLQSLSTWKDAMGNPEGYILGALANGTIFGGVLAVAFASWACDRFGRKLTTCFGSIVTVIGAILQGASTNYAFFFVSRMVIGFGFGLASVASPTLIAELSFPTYRPTCTALYNVFWYLGAVIAAWVTYGTRTIVSAYSWRIPSYLQGLLPLVQVCLVWWVPESPRFLVSKGKIEKAREFLIKFHTGNDTQEQATRLVEFELKEIEAALEMEKINSNSKYTDFITIKTFRKRIFLVAFTACMTQLSGNGLVSYYLSKVLISIGITGEKEQLQINGCLMIYNLVLSLAVAFTCYLFRRKALFIFSCSFMLLSYVIWTILSAINQQRNFEQKGLGQGVLAMIFIYYLAYNIGLNGLPYLYVTEILPYTHRAKGINLYSLVINITLIYNGFVNAIAMDAISWKYYIVYCCIIAVELVVVIFTYVETFGYTLEEVARVFEGTDSLAMDINLNGTVSNEKIDIVHSERGSSA

SEQ ID NO: 44 - protein, 563 aa, Scheffersomyces stipitis
  cellobiose transporter HXT4
MAILVESHLSLPEYRSSSNMISDNHSSSSSTEEKAAHLQYEIKSDSGELGAFSIETDFIEIEQLAQQASRKRTFWQKLLDCEFELEFKDKKHMVWLLGAFASAAGILSGVDQSIISGASIGMNTALKLTDHQSSLVSSLMPLGAMAGSMMMTPLSEYFGRKKAIVISCLWYSLGAGLCAGANSHEMMFAGRFILGIGVGIEGGSVGIYIAESVPAHVRGNLVSMYQFNIALGEVFGFAIAAIFYDIHGGWRYMVGSSLVFSTILFIGLLFLPESPRYLMYKGKVGESYNVWKRLRNADDESSKVEFLEMRHNAIIDEDRRAHESKFQVWMDLFTIPRNRRALFYAVLMVSFGQLTGINAVMYYLSTLMHKIGFNIRASVFMSLVGGGSLLIGTIPAILWMDRFGRRVWGMNIIGFFIGLVLVGVGYRFNSVTQKEAALGVYLTGLILYMSFFGAYACLTWVLPAESFSLSTRSVGMTICSTFLYLWSFTVTYNFTKMQNAFTYTGLTLGFYGGIAFIGFIYQILFMPETKDKTLEEIDDIFSKSSFQVARENISNVKRFWGFS

SEQ ID NO: 45 - protein, 189 aa, Streptomyces noursei
  nourseothricin resistance NAT1
MTTLDDTAYRYRTSVPGDAEAIEALDGSFTTDTVFRVTATGDGFTLREVPVDPPLTKVFPDDESDDESDAGEDGDPDSRTFVAYGDDGDLAGFVVVSYSGWNRRLTVEDIEVAPEHRGHGVGRALMGLATEFARERGAGHLWLEVTNVNAPAIHAYRRMGFTLCGLDTALYDGTASDGEQALYMSMPCP

SEQ ID NO: 46 - protein, 553 aa, Scheffersomyces stipitis
  glucose/xylose transporter SUT1
MSSQDIPSGVQTPSNASFLEKDEDKIEEVPQNHDATLVALESKGISEYLLICFFCLLVAFGGFVFGFDTGTISGFVNMSDFLERFGQTRADGTHYLSNVRVGLLVSIFNIGCAIGGIFLSKIGDVYGRRVGIMASMVIYVVGIIVQIASQHAWYQVMIGRAITGLAVGTVSVLSPLFIGESSPKHLRGTLVYCFQLCITLGIFIGYCVTYGTKRLSDSRQWRVPLGLCFLWAIFLVVGMLAMPESPRYLVEKKRIEDAKKSVARSNKLSPEDPSVYTEIQLIQAGIDREAIAGSASWTELITGKPAIFRRVVMGIIMQSLQQLTGVNYFFYYGTTIFQAVGLKDSFQTSIILGVVNFAATFIGIWAIERFGRRSCLLVGSAGMFVCFIIYSTIGSFHLYKDGEYNNDNTYKPSGNALIFITCLFIVFFASTWAGGVYTIISESYPLRIRSKAMAIATAANWVFGFLISFFTPFIVSAIHFKFGYVFSGCLLFSFFYVYFFVVETKGLSLEDVDELYASNVVPWKSSKWVPPSTAAMATEAGYAADEKPVDEHV

SEQ ID NO: 47 - protein, 550 aa, Scheffersomyces stipitis
  glucose/xylose transporter SUT2
MSSQDLPSGAQTPIDGSSILEDKVEQSSSSNSQSDLASIPATGIKAYLLVCFFCMLVAFGGFVFGFDTGTISGFLNMSDFLSRFGQDGSEGKYLSDIRVGLIVSIFNIGCAIGGIFLSKIGDVYGRRIGIISAMVVYVVGIIIQISSQDKWYQLTIGRGVTGLAVGTVSVLSPMFISESAPKHLRGTLVYCYQLCITLGIFIGYCVTYGTKDLNDSRQWRVPLGLCFLWAIFLVVGMLAMPESPRFLIEKKRIEEAKKSLARSNKLSPEDPGVYTEVQLIQAGIDREAAAGSASWMELITGKPAIFRRVIMGIILQSLQQLTGVNYFFYYGTTIFQAVGLQDSFQTSIILGTVNFLSTFVGIWAIERFGRRQCLLVGSAGMFVCFIIYSVIGTTHLFIDGVVDNDNTRQLSGNAMIFITCLFIFFFACTWAGGVFTIISESYPLRIRSKAMSIATAANWMWGFLISFCTPFIVNAINFKFGFVFTGCLLFSFFYVYFFVSETKGLSLEEVDELYAEGIAPWKSGAWVPPSAQQQMQNSTYGAEAKEQEQV

SEQ ID NO: 48 - protein, 550 aa, Scheffersomyces stipitis
  glucose/xylose transporter SUT3
MSSQDLPSGAQTPIDGSSILEDKVEQSSSSNSQSDLASIPATGIKAYLLVCFFCMLVAFGGFVFGFDTGTISGFLNMSDFLSRFGQDGSEGKYLSDIRVGLIVSIFNIGCAIGGIFLSKIGDVYGRRIGIISAMVVYVVGIIIQISSQDKWYQLTIGRGVTGLAVGTVSVLSPMFISESAPKHLRGTLVYCYQLCITLGIFIGYCVTYGTKDLNDSRQWRVPLGLCFLWAIFLVVGMLAMPESPRFLIEKKRIEEAKKSLARSNKLSPEDPGVYTELQLIQAGIDREAAAGSASWMELITGKPAIFRRVIMGIILQSLQQLTGVNYFFYYGTTIFQAVGLQDSFQTSIILGTVNFLSTFVGIWAIERFGRRQCLLVGSAGMFVCFIIYSVIGTTHLFIDGVVDNDNTRQLSGNAMIFITCLFIFFFACTWAGGVFTIISESYPLRIRSKAMSIATAANWMWGFLISFCTPFIVNAINFKFGFVFTGCLLFSFFYVYFFVSETKGLSLEEVDELYAEGIAPWKSGAWVPPSAQQQMQNSTYGAEAKEQEQV

SEQ ID NO: 49 - protein, 550 aa, Scheffersomyces stipitis
  glucose/xylose transporter SUT4
MSSQDLPSGAQTPIDGSSILEDKVEQSSSSNSQSDLASIPATGIKAYLLVCFFCMLVAFGGFVFGFDTGTISGFLNMSDFLSRFGQDGSEGKYLSDIRVGLIVSIFNIGCAIGGIFLSKIGDVYGRRIGIISAMVVYVVGIIIQISSQDKWYQLTIGRGVTGLAVGTVSVLSPMFISESAPKHLRGTLVYCYQLCITLGIFIGYCVTYGTKDLNDSRQWRVPLGLCFLWAIFLVVGMLAMPESPRFLIEKKRIEEAKKSLARSNKLSPEDPGVYTEVQLIQAGIDREAAAGSASWMELITGKPAIFRRVIMGIILQSLQQLTGVNYFFYYGTTIFQAVGLQDSFQTSIILGTVNFLSTFVGIWAIERFGRRQCLLVGSAGMFVCFIIYSVIGTTHLFIDGVVDNDNTRQSSGNAMIFITCLFIFFFACTWAGGVFTIISESYPLRIRSKAMSIATAANWMWGFLISFCTPFIVNAINFKFGFVFTGCLLFSFFYVYFFVSETKGLSLEEVDELYAEGIAPWKSGAWVPPSAQQQMQNSTYGAETKEQEQV

SEQ ID NO: 50 - protein, 566 aa, Scheffersomyces stipitis
  xylose transporter XUT1
MHGGGDGNDITEIIAARRLQIAGKSGVAGLVANSRSFFIAVFASLGGLVYGYNQGMFGQISGMYSFSKAIGVEKIQDNPTLQGLLTSILELGAWVGVLMNGYIADRLGRKKSVVVGVFFFFIGVIVQAVARGGNYDYILGGRFVVGIGVGILSMVVPLYNAEVSPPEIRGSLVALQQLAITFGIMISYWITYGTNYIGGTGSGQSKASWLVPICIQLVPALLLGVGIFFMPESPRWLMNEDREDECLSVLSNLRSLSKEDTLVQMEFLEMKAQKLFERELSAKYFPHLQDGSAKSNFLIGFNQYKSMITHYPTFKRVAVACLIMTFQQWTGVNFILYYAPFIFSSLGLSGNTISLLASGVVGIVMFLATIPAVLWVDRLGRKPVLISGAIIMGICHFVVAAILGQFGGNFVNHSGAGWVAVVFVWIFAIGFGYSWGPCAWVLVAEVFPLGLRAKGVSIGASSNWLNNFAVAMSTPDFVAKAKFGAYIFLGLMCIFGAAYVQFFCPETKGRTLEEIDELFGDTSGTSKMEKEIHEQKLKEVGLLQLLGEENASESENSKADVYHVEK

SEQ ID NO: 51 - protein, 551 aa, Scheffersomyces stipitis
  xylose transporter XUT3
MREVGILDVAHGNVVTIMMKDPVVFLVILFASLGGLLFGYDQGVISGIVTMESFGAKFPRIFMDADYKGWFVSTFLLCAWFGSIINTPIVDRFGRRDSITISCVIFVIGSAFQCAGINTSMLFGGRAVAGLAVGQLTMVVPMYMSELAPPSVRGGLVVIQQLSITIGIMISYWLDYGTHFIGGTRCAPSHPYQGETFNPNVDVPPGGCYGQSDASWRIPFGVQIAPAVLLGIGMIFFPRSPRWLLSKGRDEEAWSSLKYLRRKSHEDQVEREFAEIKAEVVYEDKYKEKRFPGKTGVALTLTGYWDILTTKSHFKRVFIGSAVMFFQQFIGCNAIIYYAPTIFTQLGMNSTTTSLLGTGLYGIVNCLSTLPAVFLIDRCGRKTLLMAGAIGTFISLVIVGAIVGKYGDRLSEFKTAGRTAIAFIFIYDVNFSYSWAPIGWVLPSEIFPIGIRSNAISITTSSTWMNNFIIGLVTPHMLETMKWGTYIFFAAFAIIAFFFTWLIIPETKGVPLEEMDAVFGDTAALQEKNLVTITSVSESDAKDRNSIEMSE

SEQ ID NO: 52 - protein, 318 aa, Scheffersomyces stipitis
  xylose reductase XYL1 (Xyl1p)
MPSIKLNSGYDMPAVGFGCWKVDVDTCSEQIYRAIKTGYRLFDGAEDYANEKLVGAGVKKAIDEGIVKREDLFLTSKLWNNYHHPDNVEKALNRTLSDLQVDYVDLFLIHFPVTFKFVPLEEKYPPGFYCGKGDNFDYEDVPILETWKALEKLVKAGKIRSIGVSNFPGALLLDLLRGATIKPSVLQVEHHPYLQQPRLIEFAQSRGIAVTAYSSFGPQSFVELNQGRALNTSPLFENETIKAIAAKHGKSPAQVLLRWSSQRGIAIIPKSNTVPRLLENKDVNSFDLDEQDFADIAKLDINLRFNDPWDWDKIPIFV

SEQ ID NO: 53 - protein, 363 aa, Scheffersomyces stipitis
  xylitol dehydrogenase XYL2 (PsXyl2p)
MTANPSLVLNKIDDISFETYDAPEISEPTDVLVQVKKTGICGSDIHFYAHGRIGNFVLTKPMVLGHESAGTVVQVGKGVTSLKVGDNVAIEPGIPSRFSDEYKSGHYNLCPHMAFAATPNSKEGEPNPPGTLCKYFKSPEDFLVKLPDHVSLELGALVEPLSVGVHASKLGSVAFGDYVAVFGAGPVGLLAAAVAKTFGAKGVIVVDIFDNKLKMAKDIGAATHTFNSKTGGSEELIKAFGGNVPNVVLECTGAEPCIKLGVDAIAPGGRFVQVGNAAGPVSFPITVFAMKELTLFGSFRYGFNDYKTAVGIFDTNYQNGRENAPIDFEQLITHRYKFKDAIEAYDLVRAGKGAVKCLIDGPE

SEQ ID NO: 54 - protein, 623 aa, Scheffersomyces stipitis
  xylulokinase XYL3 (PsXyl3p, PsXks1p)
MTTTPFDAPDKLFLGFDLSTQQLKIIVTDENLAALKTYNVEFDSINSSVQKGVIAINDEISKGAIISPVYMWLDALDHVFEDMKKDGFPFNKVVGISGSCQQHGSVYWSRTAEKVLSELDAESSLSSQMRSAFTFKHAPNWQDHSTGKELEEFERVIGADALADISGSRAHYRFTGLQIRKLSTRFKPEKYNRTARISLVSSFVASVLLGRITSIEEADACGMNLYDIEKREFNEELLAIAAGVHPELDGVEQDGEIYRAGINELKRKLGPVKPITYESEGDIASYFVTRYGFNPDCKIYSFTGDNLATIISLPLAPNDALISLGTSTTVLIITKNYAPSSQYHLFKHPTMPDHYMGMICYCNGSLAREKVRDEVNEKFNVEDKKSWDKFNEILDKSTDFNNKLGIYFPLGEIVPNAAAQIKRSVLNSKNEIVDVELGDKNWQPEDDVSSIVESQTLSCRLRTGPMLSKSGDSSASSSASPQPEGDGTDLHKVYQDLVKKFGDLYTDGKKQTFESLTARPNRCYYVGGASNNGSIIRKMGSILAPVNGNYKVDIPNACALGGAYKASWSYECEAKKEWIGYDQYINRLFEVSDEMNSFEVKDKWLEYANGVGMLAKMESELKH

SEQ ID NO: 55 - protein, 360 aa, Scheffersomyces stipitis
  endo-1,4-beta-xylanase XYN1
MKLEFTTALLALSGIVAANPISKNNKKHHSAPPPTLNELAVAAGKMYFGTATNQEQWSNKEYTELMLEQFGSMTPANVQKWMYTEPEQGVFNYTAGDEFANYALKNKKVLLCDTLVWHQQYPSWLDEKTWTKKDLLNVIYQHVYNEVKHFKGRCFSWNVVNEALNEDGTWRQSLFYNVTGTDYIETAFLAASAADPRAQLYYNDYNIEYPGPKSAAVENMVKWLRSKHVKIDAVGLESHFIVGQAATEAQQQQQMQSYIDLGVQVVVSELDVRFETLPPTEAGLAQQTVDYQASINACIKVGKQCMGISVWDFDDEYSWIPSSFAGQGDADLWYANFTTTPAYTGVVSALEAGALKKHIF

SEQ ID NO: 56 - DNA, 500 bp, Scheffersomyces stipitis
  terminator (1)...(500): ACB2 terminator
gtttttaatgaatagataatgtgtatgacttatcttgtgtacgtggtgactctaatatcaagaaaggacgttgtaagagagcaacgagcaaatacataatgacaaaatgtaattagtacgaacaaggaccaacattggagtctcatattcaattaaagtctgtatcatagtcaaaatctctacgctttaaatggctgcaattttatttttaaagtcacgtgatatctgaaaaatttcgagatgagaagatttatatagcatgaataaattatacccataatactctatctatcccatatatttgttcatactccatagatttcagaatggatatacatcgctgtcgttttgtggactacactccgcacaccgtcacagcaacagccttttcgcatcgttcgtcgttggctaaacaagctaccaatgatttgagattggctgttggtagaagcaatggtgacattgaaatctggaatcctaaatacaactggacccacgaa

SEQ ID NO: 57 - DNA, 300 bp, Saccharomyces cerevisiae
  terminator (1)...(300): ALD1 terminator
gcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaa

SEQ ID NO: 58 - DNA, 218 bp, Scheffersomyces stipitis
  terminator (1)...(218): BGL1 terminator
aaaatgaaatagatatggtttagaatacgttaattcggagtacttgaatctatcagtagtacaaaaacaaatgaccttattacagttcttggtttatagtgtatcaatcttcatcataaaagttcatttaaaggtataacctttttgtaaatctatagtgtctatattctaaacgttaaaaagtcatgcaggggagaaaaacattaatcgtaaagcta

SEQ ID NO: 59 - DNA, 316 bp, Scheffersomyces stipitis
  terminator (1)...(316): BGL2 terminator
tttatcagtccctaaatcggtactgcttcggggtataagaaataaatggtagttcatagaagaaatgtgggtgaatgttgttattgctcatggcaaatttagtttatcctagcagttaactccataaaaggctctatgtaatcgatgatcgtacattattcagtatttatatgtataagctatattttcgatagttgccggttcgtagatttagcattatcgattatgagcaacaatgaacaactataattaattgcattgctctcaagatatcgtcagcatagcaattctacaaccagatctattagcaacagat

SEQ ID NO: 60 - DNA, 215 bp, Scheffersomyces stipitis
  terminator (1)...(215): BGL3 terminator
aaaatgaaatagatatggtttagaatacgttaattcggagtacttgaatctatcagtagtacaaaaacaaatgacctcattacagttcttggtttatagtgtatcaatcttcatcataaaagttcatttaaaggtataacctttttgtaaatctatagtgtctatattctaaacgttaaaaagtcatgcaggggagaaaaacattaatcgtaaag

SEQ ID NO: 61 - DNA, 497 bp, Scheffersomyces stipitis
  terminator (1)...(497): BGL4 terminator
gccaattttcttacttgcagctaaaatcgcaaacttcctgacattagctatcaaacgaaaaaaacttgagctcgatccctattcacggctatcacatgaaaaagtccgcaactctttcccaaaagagaatgtaaagtctagatgatttttgtctttcgttgttcttcacaactggcaacttctttatgtagattgcaacattgaccaacactaagaaggcattttcttgctgatctcaagcatgatgccaaatatagtaattgcatggcttcacaggacatcgtagtaaacctaatctctagattttcgaatgcggcttatctgcatactgttccatacgttaatcttggattcttctatacagccacatcagaagtccttgtctggacctaataacatctaacgtgtgaaacgctatctgaagggttttcaagctagcacgtttacttcacaggagagaagctatatttttcgcttaacattttgttgttcttgcg

SEQ ID NO: 62 - DNA, 497 bp, Scheffersomyces stipitis
  terminator (1)...(497): BGL5 terminator
acgggcaatagagcctccaagagttaaaataaagttcatagttttaagtaatgtaattaaacgttgcagtaatttctgatttggctgtagatgaatatgacttccaaaactacagtctagtagacttccatcgacaaaactctcagttgaatataattggcgtatggggatatacttacaagtagaatttccattaagggaattagagttgcttcccattagtcaattttctatacaaatatatcacaataggaatcgaacccccgacttcttcgagatattctattcttctaagatttggcttaaaatgttcagaattagatatatacttctctgaaatttggaaatattgaaaagcatggaactttaaaatagacataaagcatctgcaatttcacaagatatcaaatctagtattctttttgacatttcttttcaatacaagtaaagtaactcattccactatttcttaaaacagtgctatttcataattttga

SEQ ID NO: 63 - DNA, 497 bp, Scheffersomyces stipitis
  terminator (1)...(497): BGL6 terminator
ataatttcgttaggtgttgaagctgagagtgtaatgacaaactttgtggttttaaaaaaatggtagatattactgaattaacccttctagaccaatcatctggtgttagactttcatttggagaaatattcagttgacaatatatgttctaattacaaaatttaggttttaagtgcaacaaatatatctgactttggaaaccatcggcactttcaaaattgatttgtttataaattcgtagaatatcttaatcatattgtggaatagcctgaaagtactgggagcttgttcaaaagaacatataaaaaagtggaccggaatctaatacttcagaagtttcggtggaagacttcatggtagagctagttggagacttctgtctaacaatagctcaatcatgatttctttttttattctctatctatgttttgctttactaattcggtaggaaatgctggaattcaagaaacagctagctgttgtcaatcaaattgcat

SEQ ID NO: 64 - DNA, 210 bp, Scheffersomyces stipitis
  terminator (1)...(210): BGL7 terminator
aggttttgatagaataaaaacttatatcgtaatcgttagtgcgattaatctatattagtttagccctatgagaaatgaaataagctgggttaatccctaggtatacagtttaagaaactacgtaatattatgagagatttaaattaaagaatatatattgttcacttggaataataattatgttaactcgctaatgaagaaaagagaaga

SEQ ID NO: 65 - DNA, 497 bp, Scheffersomyces stipitis
  terminator (1)...(497): EGC1 terminator
attcatacaaatcaattgaggttaggtaaagagttttgaaatttccgccacttccaaaaccacggataaaaaacattggtaaaaattatatagaatgtgaataactgaaatatacgtaaccgtgttgtttcatcatttctttgtttccaataagtttgttaccttaaggttcattatttaaattgtgttccattataatctttctatctaattttagttcaatcttatttcaatttttttcaaattctattcagaagtaaaagtattatattctaatagactggaattaattaaggcatctgggagctacttaatctagttgcaataaattcaataagatggttcccatttgaaggtcatatccgagtctatatttaccaaagaaaaatgtctagactcatagtaagtacacactgttaatattttgctatttttctaatcaggccacataaaatacaccccgtcgatattagaacattccacttactagaaattgc

SEQ ID NO: 66 - DNA, 500 bp, Scheffersomyces stipitis
  terminator (1)...(500): EGC2 terminator
agatgcaatctcgttcaatagagagatgctaggtgatgaggcgaaaaatttgactatctgactttatagagtaaaatacacataatttgtgtctaaaaactgacatatcgtaactgcttgtactgtcttgaatctcgtaactagacaattaagatgctgggtctaatccaggtgccccagaatgaaatatccctctacgagattatgtttttactgtttctctggtaactggattgcttccattttcggaaggaactccgcgacttgcagattgcctgttgcccctcctaccccagatttttgatcacacagaaaaaatatcttgagcgagatacagtggaaggtcttcgctttgggaaagcactccaacccactcttgccgttccctggatgacatccaagagtagctgaaatagacttccaccagggcaatgtatgacaaagcccaacaacaataacaataacaatattaactacttcctacagcgccctcggctgct

SEQ ID NO: 67 - DNA, 500 bp, Scheffersomyces stipitis
  terminator (1)...(500): EGC3 terminator
ctttttatttagttataaagttttaataaacatgaatgtctggttttttagttgtaactgaactgagcagagattattctttcgttattagacttctggaaggatatcaaaaagaactggcatctctcccacatgtagaaatttccccgtctactccccaacatcgaatatcgtaaattaacaatatttcaaaatggaacactctttgtataaatgtggggaattgataacattatcaacagacaattaggctttacagcacatttacttttctgactattttggagaattgcagcgataattgtaccttatctattaattataccactcaaactttctggtacttcagaggtcttccgccgaacagaaagtcgccatcaagcctctagggtcgaaattaaacttttcagatactccatctgttctagctctccattgcttacaacttcgaatatcctaactatggtacatttatattatggtacattttctataatcta

SEQ ID NO: 68 - DNA, 400 bp, Scheffersomyces stipitis
  terminator (1)...(400): FAS2 terminator
gtagatagaagattagtttattaatcgcaagtgattctatttttgattaaaaggagagtagagtgctgagagtagacagagaagtcaatgtaagcaatagaacaagagattgaacatgtctttcgaggaattccaaagcgtatatgatgtaatatgttctgtattcaagaatcagggaatagcatagttcatacatcacttgtatcctataattcactgtataagtcacagccactaattctcctatataaatgctctcgttatgtatgagaagatcagatccgagaaagagataaaatcgacacttgatatatacaccaatttattttcttatgaccccctatacttatgccgtttgataaccgatacaaagtttgtaaataacatgaattcatactat

SEQ ID NO: 69 - DNA, 547 bp, Scheffersomyces stipitis
  terminator (1)...(547): HGT1 terminator
aaaaggtcaagaatatcaagctctgtggcacgtgacacggattgacgatcctgtaatcaagctttctactgcgactcaactcaaattgaatctactgtttattccaaagagccatctaaatccctaactagcactgcttgatcctgcaacaatggcttcttataatactttgccggttgaggtaagtttttcccgtcttgaatcgcttttaatttcaattattaatagaaaatactataaattaatcaaaagatatactaactaagttgtagccttcactacttgacgaggacaatctaattcaaccagatggtacgcatgttttgctttcaactgtcctttaggaatatcttcatttacaatactaacattctttcagaaatgacagctcagccatatacgattccggattcgtctccagctccattcctggagccattacagccgagtgttaacaaaagtcaggctacgtcagcaaaatcacatatgtttcagattaacttctatcgatcgtattttaatcttgacaccgatacatttttgcaga

SEQ ID NO: 70 - DNA, 574 bp, Scheffersomyces stipitis
  terminator (1)...(574): HGT2 terminator
actaaattgatatgaataaacctgttgcaacagttgtgtgaagtcaattgttcacgtcttacaataatgtctttatgaaatgctttaaacaatgtgctatattaatttatctgtttactatcttctgtagtacttcatatacatccattatcgaagatactcttcgtaaaccaataccctaatctcgcctgtactccactgattgctgctctgctttaggtcccttcgacacttactttttgttctcgaatatatgacttgttcatcgccctaccacctaccgaatcattggtccgcaataaactgtgagctattcttgccaataaccccacgcaagattcataccaaacttttacttccatttcctattctgttctcagatagtttagtcttgtgaccccataataactagtgcttatcaattcagggccatgaaatacacaaattgctcctcattctctgaaactatcttccattttgttttgctgatgggtacacatccctttgcttcactccattttggaagaaagtggacagcaatcatctgaattcactacaccatactcaacagttc

SEQ ID NO: 71 - DNA, 337 bp, Scheffersomyces stipitis
  terminator (1)...(337): HXT2.1 terminator
agaattcatgcacaaatcagtatcttccgagagaacagaaattggttctatataagttattgtcagacttttaattttaaatgtgaaacgataaatggataaacgacttctaaattactgaatgtaaaggaaaactatcctgatttgtagaacatagctaaaaaccttgggatcgcggaagacgcgagaatccaaagaaataccaaaaatgtgttggcagaaacgacgacaagttcaaaaaaactaatataatgattctgcaatttgtaaatcgaacactttctcgtattaggataaattaattgggaactgaattatccagacattaccttatgtg

SEQ ID NO: 72 - DNA, 458 bp, Scheffersomyces stipitis
  terminator (1)...(458): HXT2.2 terminator
gtagatttttcaattctttccaatgcaaaaagaatccgttctaatgttctggaaattgctgagatgctttatataatttgtagttcatattctgatatggccgatgaataaacaagatctttgaatctttgatctgtaaaatgtagatttggcttattcttcagcgaagcaagactttatcactgtcatatgtaactgagagttttgaaaattacttaatttcacaacatttttttggaaaataccattaccagattcaaacaagagttattaatttacaaacttaagtttaggaagtcatttgttttaatataatttactcagttatagttagtttaatacgaatgcagtatttgttggaatcttgaaattgagaggaagaaccatcaattatctatatttaatcaagtttggagagtagatactttttcaaaacggtatgtatcgtgaataaagaa

SEQ ID NO: 73 - DNA, 390 bp, Scheffersomyces stipitis
  terminator (1)...(390): HXT2.3 terminator
tcaatttctatgttgaatttcaattctatagctagtctatatctggtaattaattaattctgctacaccaatatgaagttatttgagaagtcagtaaagtatcgtatcttcacaagttatttacaactgattataggaaaaattcccagatatcatccctagttaactgtgatatgtcgtagtagggcagataagtttcaagttcatactctggcctttcactgatgaccgaattggtcgtggatgtgctcagtgtctccaagtcattcataacataacttaggatattatttacaaaaataaacacggtcttaccagcaatactgacgctactaattttccaaatgatgggttggatgtcctcggtaattatgttcaagagccggatcc

SEQ ID NO: 74 - DNA, 634 bp, Scheffersomyces stipitis
  terminator (1)...(634): HXT2.4 terminator
gatagctgaacttattctaattccataatactatgagtttcaattctttaatatgctgacaaatctgtcattaattgttttttaacaacggtatatatgttctaagctttagtcaataaattatacctgattataaaatttttgctcgtttttgtaagattggattgaagtcgcctagtaaaatctacacaacataatgtcattgcataaataatcgtttattccttaaataagattcatatgcccttaagttgattaatcagtttcaaacaagacacaggctacaacaaaatcaagtgccaaagtcttgttctgtattctgctaatatattcagcacaagatttcaaaacaaaaaatgttttaagccatccatggggaaatacacatcccatcatattcagaaattcaataattgcgtccaggaatagtagtaatatgtttcgaagacacctttcgacacttatagtccatttcaggcggaaccgggcgaaagttgaaattgttttgaattctcagtctcaatacttgatcggtatttagtgttttgattgagcctgcatcaaaatagcaacttggtggcttcttctaatgtaccatttgctctcaaagttgttgcgggattaagattttatt

SEQ ID NO: 75 - DNA, 574 bp, Scheffersomyces stipitis
  terminator (1)...(574): HXT2.5 terminator
gttagttaattaggaaaactagaacccttgttagttctctacagttcagattttgatattagtgatttagtatcaagaatctagttcgaatatattttgtatgaatccataaactcaggacactaatgtaggagtatagatccaacgcaagcatgaatttaataatttttcgagtgatagcttgtctcttgatgggtcaaaaatcaaaatttttgatttgcatggtagataattatttttgtctgatcggatcaactttcaaattttgggacctagatgtattttttagcaatacttcatcttataagccatgttgccccacaaatttgttacaaatattttttgcgcgcattattccgacctaccgtagttgcaacatgaaagaccacaccatgttacatttctttagtgtgggaattaaggactgctctcccctcacttaaaaaaattgcatgcaatgagaagtgtagaatgcataaattagtttcattacctctgtgttaaaacaatataggatacatttcctacagtagagaggctgccattttcgactattccgagcgacttattttcc

SEQ ID NO: 76 - DNA, 616 bp, Scheffersomyces stipitis
  terminator (1)...(616): HXT2.6 terminator
tattaagagtgaaacctggggtaatgtttctatatttgtaaagatctcgggaaaataattcgctcggaatagtctaaaatggcagcctctctactgtaggaaatgtatcctatattgttttaacacaggggtagtgaaactaatttatgcattctacacttctcattgcatgcaatttttttaagtgaggggagagcagtccttaattcccacactaaagaaatgtaacatggtgtggtcttcaatgttgcaactaaggtaggtcggaataatgcgcgcaaaaaatatttgtaccaaatttgtggggcaacatggcttgtaagatgaagtattactaaaaaatacatctaggtcccaaaatttgaaagttgatccgatcagacaaaaataattatctaccatgcaaatcaaaaattttgacccatcaagagacaagctatcactcgaaaaattattaaattcatgcttgcgttggatctatactcctacattagtgtcctgagtttatggattcatacaaaatatattcgaactagattcttgatactaaatcactaatatcaaaatctgaactgtatagaactaacaagggttttagttttcctaattaactaac

SEQ ID NO: 77 - DNA, 500 bp, Scheffersomyces stipitis
  terminator (1)...(500): HXT4 terminator
ttagagtatttaacaatcaatcaattttgcacccgtggtattgttatctaacaaatgacgcatctaaggaaggtcgcgtcattgtataatattctgaggggtggactgactagtctaagaatgaagccttagggcccactggtagttaaatacaacacgcgtgatactttgaagagtctaggagagagttgtactgtgatatataattcttagtagagattcggtaggcttatcgatgcttttttatcgataattttagaccctgtatagcgcgaactaatttttttcgcagccattccttagatggcaagtagctagaatgaaacacactaagtatgtatgatgctaaatcaaaagaaaaacagtgaacattctccattttccaagaaccactttagagatagttcaaatatagaaacaaaaaaagtaattccgataccgggagtcgaacccgggtctgctcggtgaaagcgaaccgtgctagccgttacactatatcg

SEQ ID NO: 78 - DNA, 121 bp, Scheffersomyces stipitis
  terminator (1)...(121): SUT1 terminator
atccctaatgtcttatgcatagcattctcacgataaaaaagttatagatagtttcccttaatgtttcatagacctaatgttataaaagattgaaatcgtacgtagttcttctatgctaact

SEQ ID NO: 79 - DNA, 337 bp, Scheffersomyces stipitis
  terminator (1)...(337): SUT2 terminator
ctttacgattaatgtttttctcccctgcatgactttttaacgtttagaatatagacactatagatttacaaaaaggttatacctttaaatgaacttttatgatgaagattgatacactataaaccaagaactgtaataaggtcatttgtttttgtactactgatagattcaagtactccgaattaacgtattctaaaccatatctatttcattttctaaattccagtccagaggcattggttgtcaatatagatggattcggtttgagagatattcagactactatttccaacatggaccttgtaatttcctttttgaagggaccactgattctgat

SEQ ID NO: 80 - DNA, 336 bp, Scheffersomyces stipitis
  terminator (1)...(336): SUT3 terminator
ctttacgattaatgtttttctcccctgcatgactttttaacgtttagaatatagacactatagatttacaaaaaggttatacctttaaatgaacttttatgatgaagattgatacactataaaccaagaactgtaatgaggtcatttgtttttgtactactgatagattcaagtactccgaattaacgtattctaaaccatatctatttcattttctaaattccagtccagaggcattggttgtcaatatagatggattcggtttgagagatattcagactactatttccaacatggaccttgtaatttcctttttgaagggaccactgattctga

SEQ ID NO: 81 - DNA, 608 bp, Scheffersomyces stipitis
  terminator (1)...(608): SUT4 terminator
ctttatggttaatgctttattcccaatgatttttgaatttttaaatatagactatagatttacaaaaaggcaatgccttttaaatgaactttaatgaacgattatgataagattgatatacgacttctcggctttatagtagagtaactcaatatattatgtgctgacgaagaataaacctcaaagactttaaatggcatcaatactaactccggttatgcattatatgaatacggaacttttataaatattgatgttttatggattatataacttatatatcgttttgttaggtaagtttctaggatacttgcgaaaatgcaatgctacagcaaaaaaattcacagagtatcaatactggtacatatgattagccaccacttcgaagggtaacatttatttggtcaaagctactaataaattcaaatttatgaaaaaaacacgattgtagttactagttgtaagaaaaatgattgataacttcggactaaaattcttgaaccggaaaatccaaaaataatgcgcaaatgaacgtccctgcgccgtaagagatcaaattgcaacgaggacaaccaaaaaatgtttctcgcaactacattgatactgca

SEQ ID NO: 82 - DNA, 500 bp, Scheffersomyces stipitis
  terminator (1)...(500): TDH3 terminator
ctatccacgaagttgtaggtccactgtgtgaacctggagcttccgtgtggtgattaattacctatatattcatacatatgaattcatgaaaatgagaaatatgattagttgtagatcgtagatcgtagagagaagaattacgaagtaccgatttctgtaatggaagagttttccaacgaagaagttctagttcggtttattgacaaataaattcttttattcttgtctgacccgatgctcagctactttaccttttctactctttctactctacactgtcctttctacttctctcagttcctattcctgttcttccttttgtctcactctcatcttatctgtaacgcacctcatctcatcatagttagccacatatgacacaattgacacaattggcctgatcagagcccgaaaccatcataaaaagcaaagtccctctcgaccgaactcgctgaccaaaaatggggagtcaatggctttgtttggctcatctacatgaa

SEQ ID NO: 83 - DNA, 539 bp, Scheffersomyces stipitis
  terminator (1)...(539): TDH3 terminator
ctatccacgaagttgtaggtccactgtgtgaacctggagcttccgtgtggtgattaattacctatatattcatacatatgaattcatgaaaatgagaaatatgattagttgtagatcgtagatcgtagagagaagaattacgaagtaccgatttctgtaatggaagagttttccaacgaagaagttctagttcggtttattgacaaataaattcttttattcttgtctgacccgatgctcagctactttaccttttctactctttctactctacactgtcctttctacttctctcagttcctattcctgttcttccttttgtctcactctcatcttatctgtaacgcacctcatctcatcatagttagccacatatgacacaattgacacaattggcctgatcagagcccgaaaccatcataaaaagcaaagtccctctcgaccgaactcgctgaccaaaaatggggagtcaatggctttgtttggctcatctacatgaattactaataggtggatacccctagtcatttaaaaaacgt

SEQ ID NO: 84 - DNA, 592 bp, Saccharomyces cerevisiae
  terminator (1)...(592): TDH3 terminator
agggaaagatatgagctatacagcggaatttccatatcactcagattttgttatctaattttttccttcccacgtccgcgggaatctgtgtatattactgcatctagatatatgttatcttatcttggcgcgtacatttaattttcaacgtattctataagaaattgcgggagtttttttcatgtagatgatactgactgcacgcaaatataggcatgatttataggcatgatttgatggctgtaccgataggaacgctaagagtaacttcagaatcgttatcctggcggaaaaaattcatttgtaaactttaaaaaaaaaagccaatatccccaaaattattaagagcgcctccattattaactaaaatttcactcagcatccacaatgtatcaggtatctactacagatattacatgtggcgaaaaagacaagaacaatgcaatagcgcatcaagaaaaaacacaaagctttcaatcaatgaatcgaaaatgtcattaaaatagtatataaattgaaactaagtcataaagctataaaaagaaaatttatttaaatgcaagatttaaagtaaattcacttaagccttggc

SEQ ID NO: 85 - DNA, 147 bp, Saccharomyces cerevisiae
  terminator (1)...(147): TDH3 terminator
gtgaatttactttaaatcttgcatttaaataaattttctttttatagctttatgacttagtttcaatttatatactattttaatgacattttcgattcattgattgaaacctttgtgttttttcttgatgcgctattgcattgttct

SEQ ID NO: 86 - DNA, 300 bp, Saccharomyces cerevisiae
  terminator (1)...(300): TEF2 terminator
gagaaggagaagacagatactaaaccatacgttactcgaaacaaaaaaaaaaaaaatggaaaaagctgctatcaacaaaagacggcctcatcaaacctaaagaaaccatgtcagcgtatgtatataccttgtaatttacgtttccttaaatcttctttctactaacgttttcattattctatactctatgaccaataaaaacagactgtactttcaaaatttacccagtaggccagcaaataaagaaaattataccagattacttctgaaacacattaatcccaacaacaagtatgccat

SEQ ID NO: 87 - DNA, 300 bp, Saccharomyces cerevisiae
  terminator (1)...(300): TPI1 terminator
gattaatataattatataaaaatattatcttcttttctttatatctagtgttatgtaaaataaattgatgactacggaaagcttttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattactttgtctctaaatccttgtaaaatgtgtacgatctctatatgggttactca

SEQ ID NO: 88 - DNA, 492 bp, Scheffersomyces stipitis
  terminator (1)...(492): XUT1 terminator
tcgttctgattgagctggtctcttacagagttaataaataacctccgtatatatcaataataatactatttctactttaaaatatcgcaaattggatgtagttccaggtgcggacagtaacttaaaaaactctagagcatttggccaagcggccttcggctccttcggacttcgaaatatggaatgttcagatcatatctaggttttccaccggagtagaaattcatccgtatcatttttaagattcccgttgtccagcctgcatgttaatatgcaggggatcggaaaattagaacagatacggaattacttgatataggataattatccgttgggggataattcatttaataggaaagtgctactaattaaacttaattgtcattcctcaagtagtgtcttctgcttgtatatccttttgcactcgtaacttagccaattgaacaatcttggtaaatatgtttactgggtctgggtatctgattgaagcac

SEQ ID NO: 89 - DNA, 500 bp, Scheffersomyces stipitis
  terminator (1)...(500): XUT3 terminator
atagactggctctgaatgcgtttatataaattcaataaatgattaacgaaattaatagtcttgactacaatctcagaatcgattctttgttccaatatattatttttctgcagagttgcagccaatgcaatgcgaattaattataaggctgccaggtgcagtgctgacatcagccatgagctaagcaagtataatccacgctacaaaccaggcatgattcttaatggcaccacttcgtggcaaaagatataagaacaatattacttgctggttcctccatttatatttcaatttcggtttttccttcacaatcgatatattctactaatttccatactaatattctacaatgatcgctcaattaggtttgaacaccaagattccttaccatttcttgttctggggagttgcatttggtggttcgtctttctattcatttatcgtttctccacttgttttcaaaaagttgcctagagaagaattcagtaacttgcaaaccc

SEQ ID NO: 90 - DNA, 317 bp, Scheffersomyces stipitis
  terminator (1)...(317): XYN1 terminator
aagttcttggatatatcctggttcacttttttattttgatatatcagattacatttcgttgccaagagtacctagtgccatttctactgggcttcttttctgttcactggagtatgaaatgttcatataagtcctagttattatttcatatataggagaactttcagattcatagaatttaaatacaatttttcttatgttattattattattattattattattattattattattattattataattattattatctgcaatttctattcagtttggcaaagttattaataactactttccccaattgaaaatgt

SEQ ID NO: 91 - DNA, 625 bp, Scheffersomyces stipitis
  terminator (1)...(625): ZWF1 terminator
gtaaagacgaaaagtatgatagacgtttttggcgatgaaatgtttaatgtgatctgataatagttttgttttttctatagtttaattttgaaagtttgggcattcaatatattgatacgctttgtaactagaggtagttctagatggaagtactccacacttgtagtagtaaatgcttctgctagctctatatactttatcggcttctactggtaatactatacgcttctattcacatataattgttttgaaatctattccttcgattgtgtttccatcgggctccttcaacacagatcgtgttccaagagaatcacaccgacaacgttaaaactaaaaatcagacatccatggaaggtcgataagcggtcaccggcatactaagatgggttctattgtatgctaccgaggaaactgcgaatgtttatggtaactttcatcactttagttcaacatgtcgttgctgtattctctggaacccacgattcactagtgctttgacgaaggtgtcctttgtaaactcaacaaaagaaatggtgaaccacaaataatgaatcagaactgttataatactggcaagtataagtcccaaatcaaacacctcctgcaacagaggatcaatatt

SEQ ID NO: 92 - protein, 700 aa, Scheffersomyces stipitis
  transketolase TKL2 (Tkl2), dihydroxyacetone synthase (DHAS) (formaldehyde transketolase) (glycerone synthase)
MTQTERHKDLENYKILHPYILKVFRVLIADLVQQFNGGHPGGAMGMAAIGVALWKYVLNFSPNNPDYFNRDRFVLSNGHACLFQYAFHHLVGYKHMTMDQLKTYHSTHLESYCPGHPENEHPAIEVTTGALGQGVSNAVGLAIASKNLQATYNKPGYEVVSNHTFCMVGDACLQEGISLEAISLAGHLGLNNLTVIYDNNQVTCDGSVDLTNTENMNDKFKACNWKVIEIENGSEDVMAIVAALQKSKESSDKPTFINVHTSIGIGSNIEGQANAHGASFGEAEVDRLHQVYGFDPKNRIHIPEDVYQFFCDISSRGDILEVEWKSLVKRYGENYPELGADFARRVKGELPEDWASLIPKEFPTSDTASRASSGMIINPISSAINSLIVGTADLSPSVNLAYKDKLDFQNPRIKTTCGINGDYSGRYIHYGVREHAMAAIANGIAAFNRGTFIPCTSTFLMFYLYAAPAVRYGALSKLQVIHVATHDSIGIGEDGPTHQPIALPALYRAMPNLNYIRPCDSQEVAGAWEVAIRSKEMPTIISLSRHKLTQFPQNSKRDLVAKGAYSFHKEEDSMLNIIGVGSEMVFAVESAKLLNDRGIKTSVISFPSQYLFNKQPLEYKRSLLKRGKVPTVVIEAYTANGWERYATAGINMKTFGKSLPGPDTYRYFGFESSTIADKIEQYVAEWQTDDQIRLEFQDLN

SEQ ID NO: 93 - protein, 677 aa, Scheffersomyces stipitis
  transketolase TKT1, transketolase 1 (Tkt1)
MSSVDQKAISTIRLLAVDAVAAANSGHPGAPLGLAPAAHAVFKKMRFNPKDTKWINRDRFVLSNGHACALLYSMLVLYGYDLTVEDLKKFRQLGSKTPGHPENTDVPGAEVTTGPLGQGICNGVGIALAQAQFAATYNKPDFPISDSYTYVFLGDGCLMEGVSSEASSLAGHLQLGNLIAFWDDNKISIDGSTEVAFTEDVIARYKSYGWHIVEVSDADTDITAIAAAIDEAKKVTNKPTLVRLTTTIGFGSLAQGTHGVHGAPLKADDIKQLKTKWGFNPEESFAVPAEVTASYNEHVAENQKIQQQWNELFAAYKQKYPELGAELQRRLDGKLPENWDKALPVYTPADAAVATRKLSEIVLSKIIPEVPEIIGGSADLTPSNLTKAKGTVDFQPAATGLGDYSGRYIRYGVREHAMGAIMNGIAAFGANYKNYGGTFLNFVSYAAGAVRLSALSEFPITWVATHDSIGLGEDGPTHQPIETLAHFRATPNISVWRPADGNETSAAYKSAIESTHTPHILALTRQNLPQLEGSSIEKASKGGYTLVQQDKADIIIVATGSEVSLAVDALKVLEGQGIKAGVVSLPDQLTFDKQSEEYKLSVLPDGVPILSVEVMSTFGWSKYSHQQFGLNRFGASGKAPEIFKLFEFTPEGVAERAAKTVAFYKGKDVVSPLRSAF

SEQ ID NO: 94 - protein, 323 aa, Scheffersomyces stipitis
  transaldolase TAL1 (PsTal1p)
MSSNSLEQLKATGTVIVTDTGEFDSIAKYTPQDATTNPSLILAAAKKPEYAKVIDVAIEYAKDKGSSKKEKAEIALDRLLIEFGKNILAIVPGRVSTEVDARLSFDKEATIKKALELIALYESQGISKDRILIKIASTWEGIQAARELEAKHGIHCNLTLLFSFVQAVACAEAKVTLISPFVGRILDWYKASTGKTYEGDEDPGVISVRAIYNYYKKYGYKTIVMGASFRNTGEIKALAGCDYLTVAPKLLEELLNSTEPVPQVLDAASASATDVEKVSYVDDEATFRYLFNEDAMATEKLAQGIRAFGKDAVTLLEQLEARF